(19) United States
(12) Patent Application Publication
(10) Pub. No.: US 2022/0218259 A1
(43) Pub. Date: Jul. 14, 2022

(54) SYSTEMS AND METHODS FOR RESTRICTING RIGHTS TO AN ELECTROCARDIOGRAM PROCESSING SYSTEM

(71) Applicant: Cardiologs Technologies SAS, Paris (FR)

(72) Inventors: Johanna LAVERSIN, Montrouge (FR); Baptiste Rios CAMPO, Paris (FR); Chiara SCABELLONE, Paris (FR); Anastasiya BODROVA, Orsay (FR); Benjamin BARRE, Suresnes (FR); Gautier ZIMMERMAN, Paris (FR); Wadii HAJJI, Vitry sur Seinne (FR); Benjamin GABERNIG, Klein Sankt Paul (AT); Delphine GERMAIN, Boulogne-Billancourt (FR); Mathieu SORNAY, Paris (FR); Romain POMIER, Paris (FR); Marie-Auxille DENIS, Paris (FR); Nathan SOUFFLET, Paris (FR)

(73) Assignee: Cardiologs Technologies SAS, Paris (FR)

(21) Appl. No.: 17/657,335

(22) Filed: Mar. 30, 2022

Related U.S. Application Data
(63) Continuation-in-part of application No. 17/489,153, filed on Sep. 29, 2021.
(60) Provisional application No. 63/267,182, filed on Jan. 26, 2022.
(60) Provisional application No. 63/226,117, filed on Jul. 27, 2021.
(60) Provisional application No. 63/085,827, filed on Sep. 30, 2020.

(30) Foreign Application Priority Data
Dec. 15, 2020 (EP) 20306567.7

(51) Int. Cl. A61B 5/346; G16H 50/30; G16H 15/00; G06F 21/62

(52) U.S. Cl. CPC A61B 5/346; G06F 21/6245; G16H 15/00; G16H 50/30

(57) ABSTRACT

Systems and methods are provided for analyzing electrocardiogram (ECG) data of a patient using a substantial amount of ECG data. The systems receive ECG data from a sensing device positioned on a patient such as one or more ECG leads/electrodes that may be integrated in a smart device. The system may include an application that communicates with an ECG platform running on a server(s) that processes and analyzes the ECG data, e.g., using neural networks, to detect and/or predict various abnormalities, conditions and/or descriptors. The processed ECG data is used to generate a graphic user interface that is communicated from the server(s) to a computer for display in a user-friendly and interactive manner with enhanced accuracy. The systems may restrict access to certain ECG data, analyses, reports, and/or functionality to different entities, devices and/or users.

### CROSS-REFERENCE TO RELATED APPLICATIONS

This application claims priority to U.S. Provisional Application Ser. No. 63/267,182, filed Jan. 26, 2022, the entire contents of which are incorporated herein by reference. This application is also a continuation-in-part of U.S. patent application Ser. No. 17/489,153, filed Sep. 29, 2021, which claims priority to U.S. Provisional Application Ser. No. 63/226,117, filed Jul. 27, 2021, European Patent Application No. 20306567.7, filed Dec. 15, 2020, and U.S. Provisional Application Ser. No. 63/085,827, filed Sep. 30, 2020, the entire contents of each of which are incorporated herein by reference.

### TECHNICAL FIELD

The present disclosure relates, in general, to an electrocardiogram (ECG) processing system, for example, an ECG system with artificial intelligence and machine learning functionality for detecting and/or predicting cardiac events such as arrhythmias and abnormalities.

### BACKGROUND

An electrocardiogram (ECG) receives electrical cardiac signals from the heart that may be digitized and recorded by a computing device. An ECG typically is generated from cardiac signals sensed by a number of electrodes placed in specific areas on a patient. It is a simple, non-invasive tool, that may be used by most any healthcare professional.

A cardiac signal is composed of one or multiple synchronized temporal signals. FIG. 1A illustrates a recording of a standard 12-lead resting ECG. As is shown in FIG. 1A, each lead generates an electrical signal, resulting in 12 electrical signals. Though the ECG illustrated in FIG. 1A involves 12 leads resulting in 12 recordings, some ECGs may involve fewer leads resulting in fewer recordings. As is shown in FIG. 1A, a cardiac signal displays repeating patterns usually comprising a P-wave, a QRS complex, and a T-wave. As the name suggests, a QRS complex includes a Q-wave, an R-wave and an S-wave. An exemplary P-wave, QRS complex, and T-wave is illustrated in FIG. 1B, which focuses on a couple of beats in one lead signal, showing one R-R interval.

To make a diagnosis, a trained healthcare professional may analyze the ECG recording to identify any abnormalities and/or episodes. It is estimated that about 150 measurable abnormalities may be identified on an ECG recordings today. However, specific expertise and/or training is required to identify abnormalities from an ECG. ECG analysis is only available to those patients that can afford healthcare professions having the appropriate expertise and who otherwise have access to these professionals.

Telecardiology centers have been developed to provide ECG analysis to patients that may not otherwise have access to these trained healthcare professionals. Typically, an ECG recording is generated offsite by a non-specialist and is sent to the telecardiology center for analysis by a cardiologist or by a specialized ECG technician. While the results are generally high quality, the process may be slow and expensive.

Software systems have also been developed as an alternative to analysis by a trained professional. Current software systems provide a low quality interpretation that often results in false positives. Today, these interpretation systems may generate two types of information about a cardiac signal, (1) temporal location information for each wave, referred to as delineation, and (2) global information providing a classification of the cardiac signal or labeling its abnormalities, referred to as classification.

Concerning delineation, two main approaches are used for finding the waves of cardiac signals. The first approach is based on multiscale wavelet analysis. This approach looks for wavelet coefficients reaching predefined thresholds at specified scales. (See Martinez et al., A wavelet-based ECG delineator: evaluation on standard databases, IEEE transactions on biomedical engineering, Vol. 51, No. 4., April 2004, pp. 570-58; Almeida et al., IEEE transactions on biomedical engineering, Vol. 56, No. 8, August 2009, pp 1996-2005; Boichat et al., Proceedings of Wearable and Implantable Body Sensor Networks, 2009, pp. 256-261; U.S. Pat. No. 8,903,479 to Zoicas et al.). The usual process involves identifying QRS complexes, then P-waves, and finally T-waves. This approach is made unstable by the use of thresholds and fails to identify multiple P-waves and “hidden” P-waves.

The second delineation approach is based on Hidden Markov Models (HMM). This machine learning approach treats the current state of the signal as a hidden variable that one wants to recover (Coast et al., IEEE transactions on biomedical engineering, Vol. 37, No. 9, September 1990, pp 826-836; Hughes et al., Proceedings of Neural Information Processing Systems, 2004, pp 611-618; U.S. Pat. No. 8,332,017 to Trassenko et al.). While this approach is an improvement upon on the first delineation approach described above, a representation of the signal must be designed using handcrafted “features,” and a mathematical model must be fitted for each wave, based on these features. Based on a sufficient number of examples, the algorithms may learn to recognize each wave. This process may however be cumbersome and inaccurate due to its dependence on handcrafted features. Specifically, features which have been handcrafted will always be suboptimal since they were not learnt and the process of handcrafting features may have ignored or eliminated crucial information. Further, the model, usually Gaussian, is not well adapted. Also, the current models fail to account for hidden P waves.

Regarding classification, in current systems analysis is only performed on the QRS complex. For example, analysis of a QRS complex may detect ventricular or paced beats. The training involves handcrafted sets of features and corresponding beat labels (Chazal et al., IEEE Transactions on Biomedical Engineering, 2004, vol. 51, pp. 1196-1206). As explained above, features that have been handcrafted will always be suboptimal since they were not learnt and the process of handcrafting features may have ignored or eliminated crucial information.

To solve the above issues, recent works (Kiranyaz et al., IEEE Transactions on Biomedical Engineering, 2016, Vol. 63, pp 664-675) have turned to novel architectures called neural networks which have been intensively studied and had great results in the field of imaging (Russakovsky et al., arXiv: 1409.0575v3, 30 Jan. 2015). Neural networks learn from raw or mildly preprocessed data and thus bypass the need of handcrafted features. While the application of neural networks is an improvement on the delineation and classification approaches described above, current systems have certain drawbacks. For example, the current neural networks were only developed for QRS characterization. Further, current neural networks processes information in a beat-by-beat manner which fails to capture contextual information from surrounding beats.

Concerning identifying abnormalities and/or cardiovascular disease detection, most algorithms use rules based on temporal and morphological indicators computed using the delineation (e.g., PR interval, RR interval, QT interval, QRS width, level of the ST segment, slope of the T-wave). Often times, the algorithms are designed by cardiologists. (Prineas et al., The Minnesota Code Manual of Electrocardiographic Findings, Springer, ISBN 978-1-84882-777-6, 2009). However, the current algorithms do not reflect the way the cardiologists analyze the ECGs and are crude simplifications. For example, the Glasgow University Algorithm does not reflect the way cardiologist analyze ECGs. (Statement of Validation and Accuracy for the Glasgow 12-Lead ECG Analysis Program, Physio Control, 2009.)

More advanced methods have also been developed that use learning algorithms. In. Shen et al., Biomedical Engineering and Informatics (BMEI), 2010. vol. 3, pp. 960-964, for instance, the author used support vector machines to detect bundle branch blocks. However, in these methods, once again, it is necessary to represent the raw data in a manner that preserves the invariance and stability properties.

While more complex neural network architectures have been proposed, limitations arose when they were applied to ECGs. One team (Jin and Dong, Science China Press, Vol. 45, No 3, 2015, pp 398-416; CN104970789) proposed binary classification on a full ECG, hence providing one and only one classification for any analyzed ECG. The proposed architecture used convolutional layers which processes the leads independently before mixing them into fully connected layers. The authors also mention multi-class analysis, as opposed to binary analysis, aiming at recovering one class among several. However, they did not consider multi-label classification, wherein multiple labels (e.g., abnormalities) are assigned to a cardiac signal.

Other algorithms and neural network architectures have been proposed to detect the risk of atrial fibrillation. In Attia et al., “An artificial intelligence-enabled ECG algorithm for the identification of patients with atrial fibrillation during sinus rhythm: a retrospective analysis of outcome prediction,” The Lancet, Volume 394, Issue 10201, P861-867, Sep. 7, 2019, the entire contents of which are incorporated herein by reference, the author describes using artificial intelligence and convolutional neural networks to detect asymptomatic atrial fibrillation.

In view of the foregoing limitations of previously-known systems and methods, it would be desirable to accurately and efficiently process ECG data and to present this information in a way that is easily comprehendible. For example, it would be desirable to use enhanced computing technology to analyze ECG data sampled from a patient to accurately and efficiently detect and/or predict cardiac events, e.g., using artificial intelligence and/or machine learning technology specifically designed for ECG analysis.

### SUMMARY OF THE INVENTION

Provided herein are systems and methods for analyzing ECG data using machine learning algorithms and medical grade artificial intelligence with enhanced accuracy and efficiency. Specifically, systems and methods are provided for analyzing electrocardiogram (ECG) data of a patient using artificial intelligence and a substantial amount of ECG data. The systems receive ECG data from a sensing device positioned on a patient such as one or more ECG leads/electrodes that may be integrated into smart technology (e.g., a smartwatch). The system may analyze ECG data sampled from the patient to accurately and efficiently detect and/or predict cardiac events such as such as cardiac arrhythmias and/or abnormalities including atrial fibrillation (AFib). The system may include an application that communicates with an ECG platform running on a server that processes and analyzes the ECG data, e.g., using neural networks for delineation of the cardiac signal and classification of various abnormalities, conditions and/or descriptors. The ECG platform may be a cloud-based ECG platform that processes and analyzes the ECG data in the cloud. The processed ECG data is communicated from the server for display in a user-friendly and interactive manner with enhanced accuracy. Together the ECG application and ECG platform implement the ECG processing system to receive ECG data, process and analyze ECG data, display ECG data on a system device, and generate a report having ECG data.

A computerized-method is provided herein for analyzing electrocardiogram (ECG) data of a patient and restricting access to analyzed ECG data. The method may involve receiving, by a server, authorization instructions corresponding to a first location on the server. The method may further involve receiving, based on the authorization instructions, a set of patient ECG data from a first user account accessed using a first device. The method may further involve storing the set of patient ECG data at the first location on the server. The method may further involve processing at least a portion of the set of patient ECG data using an algorithm to determine a presence of one or more abnormalities, conditions, or descriptors corresponding to a cardiac event associated with the set of patient ECG data, the algorithm trained using a plurality of sets of ECG data different from the set of ECG data. The method may further involve generating output data based on the presence of the one or more abnormalities, conditions, or descriptors. The method may further involve storing the output data at the first location. The method may further involve receiving a request to access the output data from a second user account accessed using the first device or a second device. The method may further involve permitting the second user account to access to the output data based on the authorization instructions. The authorization instructions are received from the first user account or the second user account. The authorization instructions comprise at least one of: authorization to access the first location, authorization to upload data to the first location, authorization to access a first type of file within the first location, authorization to access or revise administrative information, authorization to view the output data, authorization to revise the output data, or authorization to generate a report based on the output data

The method may further involve determining the second user account has authorization to access the set of patient ECG data based on the authorization instructions. The method may further involve permitting, based on determining the second user account has authorization to access the set of patient ECG data, access to the patient ECG data to the second user account. The method may further involve receiving, from the second user account, a request to process the set of patient ECG data using the algorithm. The method may further involve receiving, from the first user account, a request to process the set of patient ECG data using the algorithm. The method may further involve receiving, from the second user account, a request to generate a report based on the output data. The method may further involve determining the second user account has authorization to access the report saved at the first location based on the authorization instructions. The method may further involve sending, upon receiving the set of patient ECG data and storing the patient ECG data at the first location, a message to the second user account indicating that the set of patient ECG data is saved at the first location. The computerized-method may further include receiving a request from one or more of the first account and the second account to view the output data, and granting the request to view the output data based on the authorization instructions. The computerized method may further including receiving a request to modify the output data from one or more of the first account and the second account, and granting the request to modify the output data based on the authorization instructions.

A computerized system is described herein. The computerized system may be used for analyzing electrocardiogram (ECG) data of a patient and restricting access to analyzed ECG data. The computerized system may be designed to receive, by a server, authorization instructions corresponding to a first location on the server. The computerized system may be further designed to receive, based on the authorization instructions, a set of patient ECG data from a first user account accessed using a first device. The computerized system may be further designed to store the set of patient ECG data at the first location on the server. The computerized system may be further designed to processing at least a portion of the set of patient ECG data using an algorithm to determine a presence of one or more abnormalities, conditions, or descriptors corresponding to a cardiac event associated with the set of patient ECG data, the algorithm trained using a plurality of sets of ECG data different from the set of ECG data. The computerized system may be further designed to generate output data based on the presence of the one or more abnormalities, conditions, or descriptors. The computerized system may be further designed to store the output data at the first location. The computerized system may be further designed to receive a request to access the output data from a second user account accessed using the first device or a second device. The computerized system may be further designed to permit the second user account to access to the output data based on the authorization instructions. The authorization instructions are received from the first user account or the second user account. The authorization instructions comprise at least one of: authorization to access the first location, authorization to upload data to the first location, authorization to access a first type of file within the first location, authorization to access or revise administrative information, authorization to view the output data, authorization to revise the output data, or authorization to generate a report based on the output data

The computerized system may be further designed to determine the second user account has authorization to access the set of patient ECG data based on the authorization instructions. The computerized system may be further designed to permit, based on determining the second user account has authorization to access the set of patient ECG data, access to the patient ECG data to the second user account

The computerized system may be further designed to receive, from the second user account, a request to process the set of patient ECG data using the algorithm. The computerized system may be further designed to receive, from the first user account, a request to process the set of patient ECG data using the algorithm. The computerized system may be further designed to receive, from the second user account, a request to generate a report based on the output data. The computerized system may be further designed to determine the second user account has authorization to access the report saved at the first location based on the authorization instructions. The computerized system may be further designed to send, upon receiving the set of patient ECG data and storing the patient ECG data at the first location, a message to the second user account indicating that the set of patient ECG data is saved at the first location. The computerized system may be further designed to include receiving a request from one or more of the first account and the second account to view the output data, and granting the request to view the output data based on the authorization instructions. The computerized system may further be designed to include receiving a request to modify the output data from one or more of the first account and the second account, and granting the request to modify the output data based on the authorization instructions.

A non-transitory computer-readable medium is described herein. The non-transitory computer-readable medium may include computer-executable instructions, that when executed by at least one processor, cause the at least one processor to receive, by a server, authorization instructions corresponding to a first location on the server. The computer-executable instructions may further cause the at least one processor to receive, based on the authorization instructions, a set of patient ECG data from a first user account accessed using a first device. The computer-executable instructions may further cause the at least one processor to store the set of patient ECG data at the first location on the server. The computer-executable instructions may further cause the at least one processor to processing at least a portion of the set of patient ECG data using an algorithm to determine a presence of one or more abnormalities, conditions, or descriptors corresponding to a cardiac event associated with the set of patient ECG data, the algorithm trained using a plurality of sets of ECG data different from the set of ECG data. The computer-executable instructions may further cause the at least one processor to generate output data based on the presence of the one or more abnormalities, conditions, or descriptors. The computer-executable instructions may further cause the at least one processor to store the output data at the first location. The computer-executable instructions may further cause the at least one processor to receive a request to access the output data from a second user account accessed using the first device or a second device. The computer-executable instructions may further cause the at least one processor to permit the second user account to access to the output data based on the authorization instructions The authorization instructions are received from the first user account or the second user account.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1A is a recording of a standard 12-lead resting ECG and FIG. 1B is a recording of an exemplary P-wave, QRS complex and T-wave.

FIG. 2 is a diagram illustrating exemplary components for executing systems and methods in accordance with aspect of the present disclosure.

FIGS. 3A-3B are schematic views of the exemplary hardware and software components of an exemplary system device and an exemplary server, respectively.

FIG. 4 is a flow chart of an exemplary method of processing ECG data using, displaying ECG data, and generating a report including ECG data.

FIGS. 5A-5B are line graphs representing an exemplary ECG signal and an exemplary output of a first neural network for each wave type analyzed, respectively.

FIGS. 6A-6B are exemplary representations of classification neural networks in the form of a convolutional neural network and a recurrent neural network, respectively.

FIG. 7 is an exemplary representation of a variable number of lead entries and a constant number of outputs.

FIG. 8 is an exemplary user interface having a heart rate density plot generated in accordance with aspects of the recent disclosure.

FIG. 9 is a zoomed-in view of the heart rate density plot shown in FIG. 8.

FIG. 10 is an exemplary user interface having a heart rate density plot generated in accordance with aspects of the present disclosure.

FIG. 11 is a flow chart illustrating an exemplary approach for generating a heart rate density plot.

FIG. 12 is an exemplary heart rate density plot generated in accordance with aspects of the present disclosure.

FIG. 13 is an exemplary user interface having a zoomed-in heart rate density plot.

FIGS. 14A-14E are side-by-side comparisons of various R-R plots and heart rate density plots generated from the same cardiac signal.

FIGS. 15A-15D is an exemplary report generated by the ECG processing system having information corresponding to the patient and processed ECG data and displaying a heart rate density plot and ECG strips.

FIG. 16 illustrates an exemplary process flow for determining ECG data and associating the ECG data to a user profile.

FIGS. 17A-17B illustrate an exemplary process and data flow for determining ECG data, parsing the ECG data, and determining reports based on the ECG data.

FIG. 18 illustrates an exemplary process flow for determining ECG data, determining a report, prioritizing the report, and signing the report.

FIGS. 19A-19C illustrate an exemplary ILR event monthly summary report.

FIG. 20 illustrates an exemplary ILR event report.

FIGS. 21A-21C illustrate an exemplary monthly report and events list user interface.

FIGS. 22A-22B illustrate exemplary user registration and profile interfaces.

FIG. 23A illustrates an exemplary event interface including a reclassification menu. FIG. 23B illustrates an exemplary process for reclassifying an event.

FIG. 24 illustrates color bands that may be displayed on an event interface.

FIG. 25A is a diagram illustrating an exemplary multi-user device system for analyzing ECG and other data.

FIG. 25B illustrates a process for analyzing ECG data and other data to determine an anomaly, descriptor, or condition using multiple user devices.

FIG. 25C illustrates a process for analyzing ECG data and other data to determine an anomaly, descriptor, or condition using multiple user devices.

FIG. 25D illustrates a user interface for displaying ECG data and other data.

FIG. 25E illustrates a user interface for displaying additional information relating to individual ECG data points and/or other data points.

FIG. 25F illustrates a user interface for displaying an ECG representation corresponding to ECG data points and/or other data points.

FIG. 25G illustrates an exemplary mobile device interface for presenting heart rate and/or ECG data and results.

FIG. 26 illustrates an exemplary mobile device interface for presenting ECG data and results.

FIG. 27 is an exemplary process for prioritizing certain data for review by the healthcare provider based on a user indication.

FIG. 28. is an exemplary process for determining a time period for which an arrhythmia is likely and determining ECG data during that time period.

FIG. 29. is an exemplary process for determining a time period for which atrial fibrillation is likely based on the PAC burden and determining ECG data during that time period.

FIGS. 30A-30B illustrate an events report including a graphical representation of events detected.

FIGS. 31A-31F illustrate various user interfaces for displaying patients, indications, classifications and/or events.

FIG. 32 is a portion of an ECG report including selectable ECG strips and selectable links to be redirected to a viewer application.

FIG. 33 illustrates a viewer interface of a viewer application including a heart rate density plot and ECG strips.

FIG. 34 is an exemplary process for redirecting a user from the report to a viewer application including a viewer interface.

FIGS. 35A-35C are exemplary report, patients and event list interfaces.

FIG. 36 is a diagram illustrating exemplary components for executing systems and methods in accordance with an aspect of the present disclosure.

FIGS. 37A-37B are exemplary processes for providing and accessing ECG analyses.

FIGS. 38A-38B are exemplary processes for providing and accessing ECG analyses.

FIG. 39 is an exemplary user interface for an account manager.

FIG. 40 is an exemplary user interface for reports.

FIG. 41 is an exemplary user interface for selecting rights and/or access.

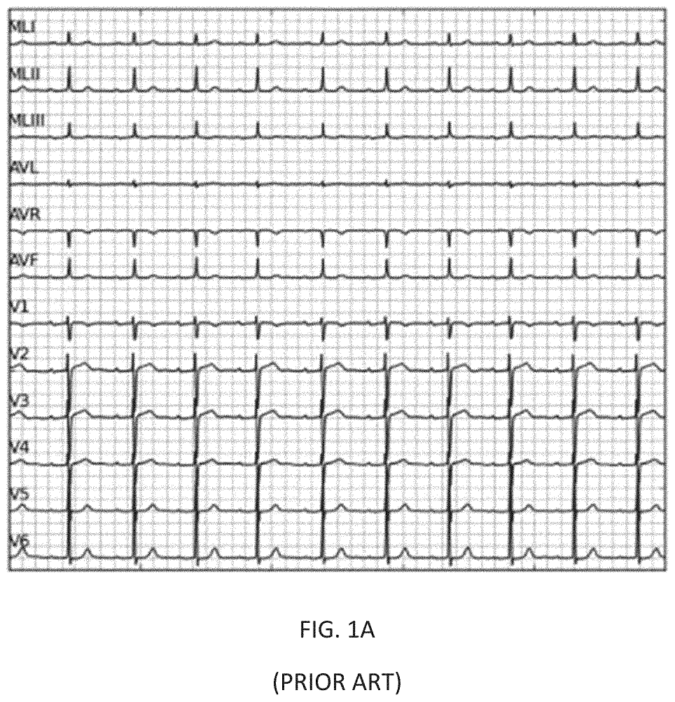

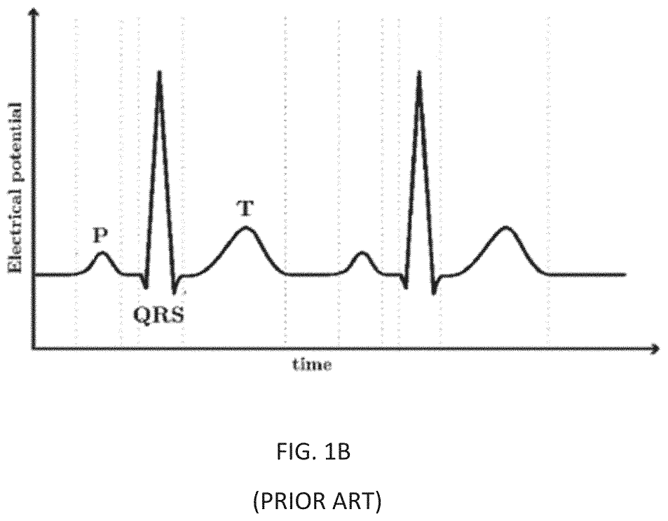

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION OF THE INVENTION

The present invention is directed to an electrocardiogram (ECG) processing system having medical grade artificial intelligence involving an ECG application run on a system device and an ECG platform run on a server(s). The ECG application and ECG platform implement the ECG processing system by processing and analyzing the ECG data using machine learning algorithms to detect and/or predict cardiac events such as such as cardiac arrhythmias and/or abnormalities including atrial fibrillation (AFib). The system may achieve delineation of the cardiac signal and classification of various abnormalities, conditions, and descriptors. The server(s) may be located in a different location than the system device(s) and the servers need not be in the same physical location as one another (e.g., the server(s) may be a remote server(s)). Alternatively, the server(s) and the system device(s) may be located in the same general area (e.g., on a local area network (LAN)). The ECG platform may be a cloud-based ECG platform that may implement the ECG processing system by processing and analyzing the ECG data in the cloud.

To implement the ECG processing system, ECG application running on the system device may receive ECG data (i.e., cardiac signal) from a sensing device and may transmit the ECG data to a ECG platform running on the server. The ECG platform may execute a first and second neural network and may apply the ECG data to the first and second neural network. The first neural network may be a delineation neural network having machine learning functionality. The second neural network may be a classification neural network having machine learning functionality. The output of the first and/or second neural networks may be processed by the ECG platform to achieve delineation and classification of the ECG data. The ECG data and/or data generated by the ECG platform may be communicated from the ECG platform to the ECG application. The ECG application may cause the ECG data and/or data generated by the ECG platform to be displayed in an interactive manner. The ECG platform may generate reports including ECG data and/or data generated by the ECG platform, and may communicate the reports to the ECG application.

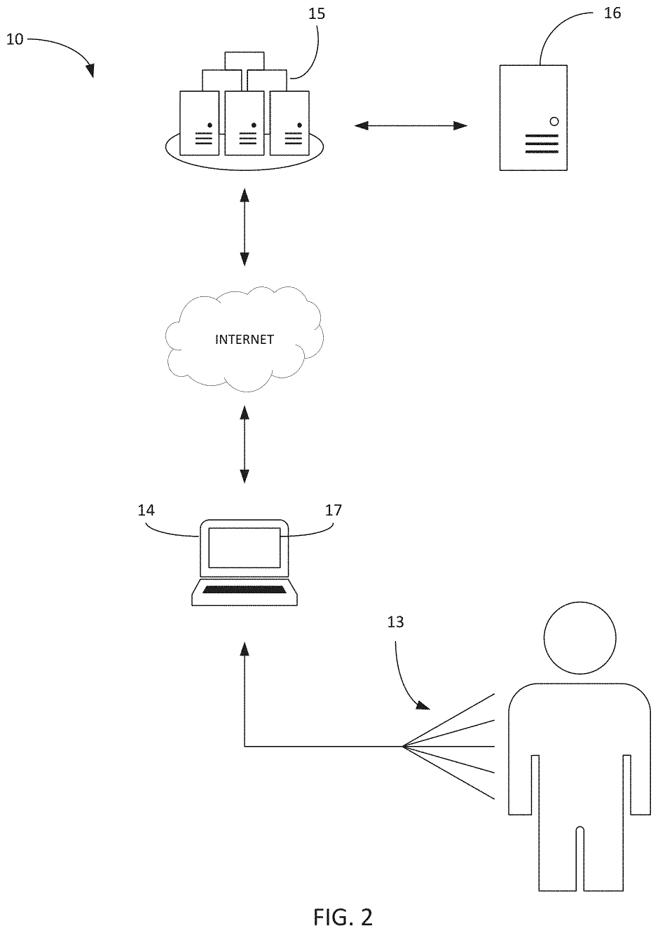

Referring now to FIG. 2, exemplary components for executing electrocardiogram (ECG) processing system 10 are illustrated. FIG. 2 shows ECG sensing device 13, system device 14, and server 15, as well as drive 16.

ECG sensing device 13 is designed to sense the electrical activity of the heart for generating ECG data. For example, sensing device 13 may be one or more electrodes that may be disposed on one or more leads. ECG sensing device 13 may be an ECG-dedicated sensing device such as a conventional 12-lead arrangement or may be a multi-purposes device with sensing hardware for sensing electrical activity of the heart for ECG generation such as the Apple Watch available from Apple, Inc., of Cupertino, Calif. Sensing device 13 may be placed on the surface of the chest of a patient and/or limbs of a patient. Sensing device 13 may be in electrical communication with system device 14 running the ECG application 29 such that the electrical signal sensed by sensing device 13 may be received by the ECG application 29. ECG application 29 may include instructions that cause sensing device 13 to sense or otherwise obtain ECG data.

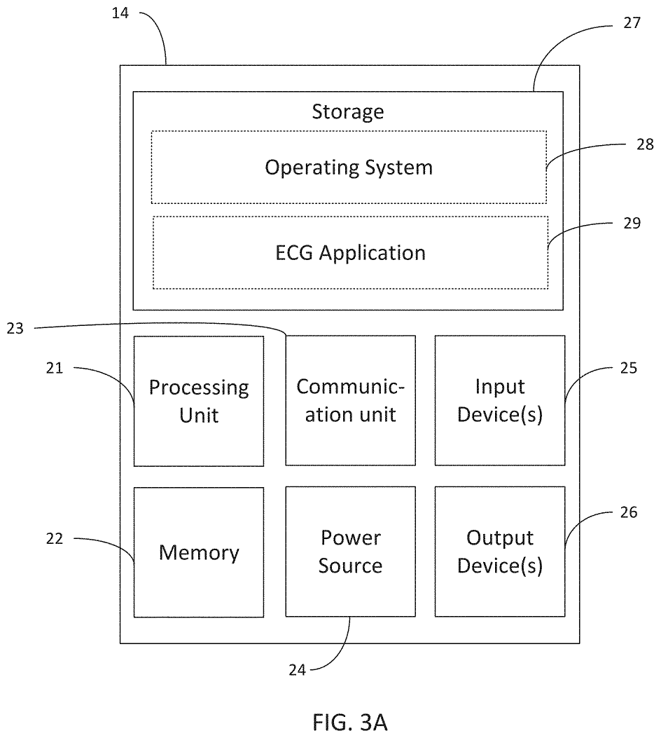

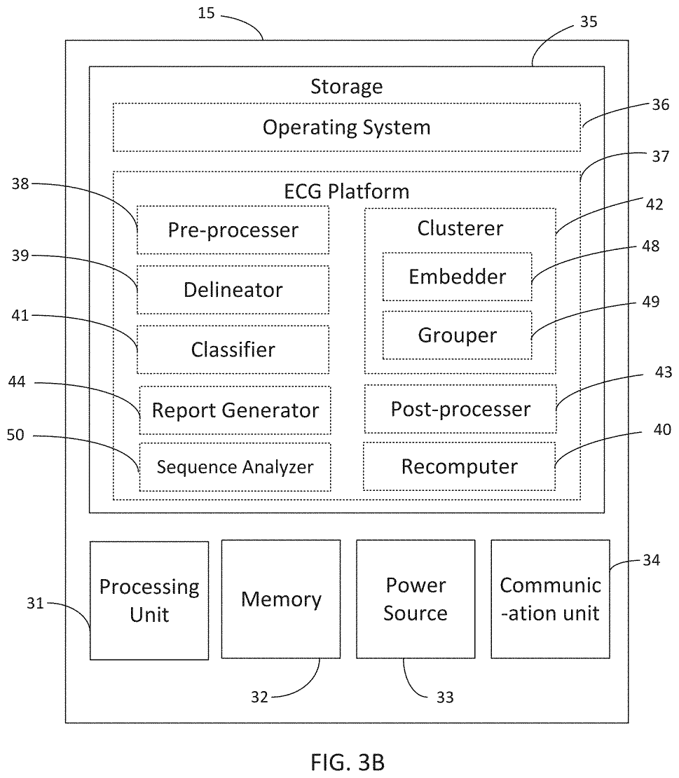

System device 14 is preferably one or more computing devices (e.g., laptop, desktop, tablet, smartphone, smartwatch, etc.) having the components described below with reference to FIG. 3A and the functionality described herein. System device 14 running ECG application 29 may connect with server 15 running ECG platform 37 via any well-known wired or wireless connection. For example, system device 14 may connect to the Internet using well known technology (e.g., WiFi, cellular, cable/coaxial, and/or DSL) and may communicate with server 15 over the Internet.

Server 15 is preferably one or more servers having the components described below with reference to FIG. 3B and the functionality described herein. Server 15 preferably has processing power superior to system device 14 such that server 15 can process and analyze cardiac signals having a sampling rate above a predetermined threshold, such as at least 20 samples per second, at least 250 samples per second, or at least 1000 samples per second. As will be readily apparent to one skilled in the art, server 15 may include a plurality of servers located in a common physical location or in different physical locations. In a preferred embodiment, server 15 is located in a different, remote location (e.g., on the cloud) than system device 14, although server 15 and system device 14 may be located in a common location (e.g., on a local area network (LAN)).

Server 15 may optionally communicate with drive 16 which may be one or more drives having memory dedicated to storing digital information unique to a certain patient, professional, facility and/or device. For example, drive 16 may include, but is not limited to, volatile (e.g. random-access memory (RAM)), non-volatile (e.g. read-only memory (ROM)), flash memory, or any combination thereof. Drive 16 may be incorporated into server 15 or may be separate and distinct from server 15 and may communicate with server 15 over any well-known wireless or wired connection.

Aspects of ECG processing system 10 and/or any other ECG processing systems described throughout this application may be the same or similar to the ECG processing system described in WO2020161605A1, which is the published application of PCT/IB2020/050850, filed on Feb. 3, 2020, (corresponding to U.S. Ser. No. 17/390,714), which claims priority to U.S. Pat. No. 10,959,660 to Li, the entire contents of each of which are incorporated herein by reference. Additional technology that may be utilized is described in commonly-assigned U.S. Ser. No. 17/397,782, the entire contents of which are incorporated herein by reference.

Referring now to FIGS. 3A-3B, exemplary functional blocks representing the hardware and software components of system device 14 and server 15 are shown. Referring now to FIG. 3A, hardware and software components of system device 14 may include one or more processing unit 21, memory 22, storage 27, communication unit 23, and power source 24, input devices 25 and output devices 26.

Processing unit 31 may be one or more processors configured to run collaboration operating system 28 and ECG application 29 and perform the tasks and operations of system device 14 set forth herein. Memory 22 may include, but is not limited to, volatile (e.g. random-access memory (RAM)), non-volatile (e.g. read-only memory (ROM)), flash memory, or any combination thereof. Communication unit 23 may receive and/or transmit information to and from other components in ECG processing system 10 including, but not limited to, sensing device 13 and server 15. Communication unit 23 may be any well-known communication infrastructure facilitating communication over any well-known wired or wireless connection, including over any well-known standard such as any IEEE 802 standard. Power source 24 may be a battery or may connect system device 14 to a wall outlet or any other external source of power. Storage 27 may include, but is not limited to, removable and/or non-removable storage such as, for example, magnetic disks, optical disks, or tape.

Input device 25 may be one or more devices coupled to or incorporated into system device 14 for inputting data to system device 14. Input device 25 may further include a keyboard, a mouse, a pen, a sound input device (e.g., microphone), a touch input device (e.g., touch pad or touch screen), a location sensor, and/or a camera, for example. Output device 26 may be any device coupled to or incorporated into system device 14 for outputting or otherwise displaying data and includes at least a display 17. Output device 26, may further include speakers and/or a printer, for example.

ECG application 29 may be stored in storage 27 and executed on processing unit 21. ECG application 29 may be a software application and/or software modules having one or more sets of instructions suitable for performing the operations of system device 14 set forth herein, including facilitating the exchange of information with sensing device 13 and server 15. For example, ECG application 29 may cause system device 14 to receive ECG data from sensing device 13, to record ECG data from sensing device 13, to communicate ECG data to server 15, to instruct server 15 to process and analyze ECG data, to receive processed and/or analyzed ECG data from server 15, to communicate user input regarding report generation to server, and to generate a graphic user interface suitable for displaying raw, analyzed and/or processed ECG data and data related thereto.

Operating system 28 may be stored in storage 27 and executed on processing unit 21. Operating system 28 may be suitable for controlling the general operation of system device 14 and may work in concert with ECG application 29 to achieve the functionality of system device 14 described herein. System device 14 may also optionally run a graphics library, other operating systems, and/or any other application programs. It of course is understood that system device 14 may include additional or fewer components than those illustrated in FIG. 3A and may include more than one of each type of component.

Referring now to FIG. 3B, hardware and software components of server 15 may include one or more processing unit 31, memory 32, storage 35, power source 33, and communication unit 34. Processing unit 31 may be one or more processors configured to run operating system 36 and ECG platform 37 and perform the tasks and operations of server 15 set forth herein. Given the volume of data and processing tasks assigned to processing unit 31, it is understood that processing unit 31 has superior processing power compared to processing unit 21.

Memory 32 may include, but is not limited to, volatile (e.g. random-access memory (RAM)), non-volatile (e.g. read-only memory (ROM)), flash memory, or any combination thereof. Storage 35 may include, but is not limited to, removable and/or non-removable storage such as, for example, magnetic disks, optical disks, or tape. Communication unit 34 may receive and/or transmit information to and from other components of ECG processing system 10 including, but not limited to, system device 14 and/or drive 16. Communication unit 34 may be any well-known communication infrastructure facilitating communication over any well-known wired or wireless connection. Power source 33 may be a battery or may connect server 15 to a wall outlet or other external source of power.

Operating system 36 and ECG platform 37 may be stored in storage 35 and executed on processing unit 31. Operating system 36 may be suitable for controlling general operation of server 15. ECG platform 37 may be a software application and/or software modules having one or more sets of instructions. ECG platform 37 may facilitate and oversee the processing and analysis of ECG data received from system device 14, report generation, and otherwise may be suitable for performing the operations of server 15 set forth herein.

ECG platform 37 may include several sub-modules and/or applications including, but not limited to, pre-processor 38, delineator 39, classifier 41, clusterer 42 which may include embedder 48 and grouper 49, post-processor 43, report generator 44, recomputer 40 and/or sequence analyzed 50. Each sub-module and/or application may be a separate software application and/or module having one or more sets of instructions. Pre-processor 38 may pre-process raw ECG data, delineator 39 may execute a first neural network to achieve delineation, classifier 41 may execute a second neural network to achieve classification, clusterer 42 may identify clusters in data processed by the first neural network, post-processor 43 may post-process data processed by the second neural network, embedder 48 may execute one or more algorithms and/or a third neural network to achieve embedding, grouper 49 may execute one or more algorithms and/or a fourth neural network to generate cluster groups, report generator 44 may generate reports based on raw ECG data and ECG data processed by ECG platform 37, and recomputer 40 may recompute and/or adjust embedder 48 and/or grouper 49 based on user input data. For example, recomputer 40 may recalculate episodes based on corrected wave information. Sequence analyzer 50 may be one or more algorithms and/or a third neural network which may be a recurrent neural network. Sequence analyzer 50 may analyze feature maps to determine one or more sequence labels and thereby achieve sequence identification as explained below. ECG platform 37 may also perform various other functions including, but not limited to, receiving requests from system device 14 to process and/or analyze ECG data, communicating processed and/or analyzed ECG data to system device 14, receiving a request to generate a report, requesting and/or receiving user interaction and/or instructions from system device 14, receiving user input data and/or instruction information from system device 14 regarding report generation, and/or communicating a report to system device 14.

Server 15 may also optionally run a graphics library, other operating systems, and/or any other application programs. It of course is understood that server 15 may include additional or fewer components than those illustrated in FIG. 3B and may include more than one of each type of component.

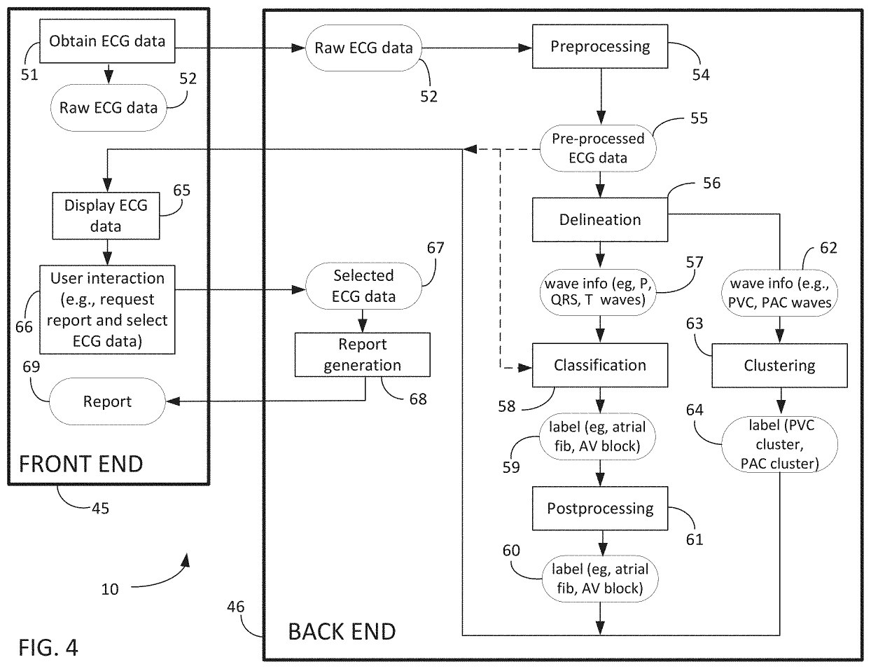

FIG. 4 illustrates an exemplary process for implementing ECG processing system 10 to receive and record ECG data, process and analyze ECG data, and generate reports involving ECG data, and further shows the flow of information between front end 45 and back end 46 of ECG processing system 10, as described, for example, in U.S. Patent Pub. Nos. 2019/0167143, U.S. Patent Pub. No. 2019/0223739, and U.S. Pat. No. 10,426,364, the entire contents of each of which are incorporated herein by reference. Front end 45 includes at least ECG application 29 running on system device 14. Back end 46 includes at least ECG platform 37 running on server 15.

As is shown in FIG. 4, at step 51, ECG application 29 may cause system device 14 to receive and/or otherwise obtain raw ECG data 52 from sensing device 13. For example, ECG application 29 may cause sensing device 13 to sense the cardiac signal and communicate the cardiac signal sensed by sensing device 13 to system device 14. Raw ECG data is the cardiac signal sensed by sensing device 13. Raw ECG data 52 has not been processed or analyzed by ECG processing system 10. Raw ECG data 52 preferably involves data sampled multiple times per heartbeat over a plurality of heartbeats. It is understood that sensing device 13 may convert an analog cardiac signal into a digital signal, a different component not shown in FIG. 2 may convert the analog cardiac signal to a digital signal, or ECG application 29 may cause system device 14 to convert the analog cardiac signal to a digital signal. Raw ECG data in both analog and digital form are referred to herein as raw ECG data 52.

Upon receiving raw ECG data 52, ECG application 29 may cause system device 14 to record raw ECG data 52 and may optionally save some or all of raw ECG data 52 to system device 14. As explained above, the signals may correspond to one or more leads. When multiple leads are used, all leads may be processed simultaneously. It is understood that the cardiac signal generated by each lead may have varying lengths. It is further understood that the cardiac signal may be short term (e.g., 10 seconds in standard ECGs) or long term (several days in holters). System device 14 may optionally display raw ECG data 52 or a portion thereof on display 17.

As is shown in FIG. 4, raw ECG data 52 may be transmitted from front end 45 to back end 46. Specifically, ECG application 29 may cause system device 14 to communicate raw ECG data 52 to ECG platform 37 running on server 15. Upon receiving raw ECG data 52, ECG platform 37 may cause server 15 to save some or all of raw ECG data 52 to server 15. Further, after receiving raw ECG data 52, ECG platform 37 cause raw ECG data 52 to be preprocessed at step 54 by pre-processor 38. It is understood that pre-processor 38 may be a stand-alone component of ECG platform 37 or subcomponent of delineator 39.

Pre-processor 38 may process raw ECG data 52 or a portion thereof by removing the disturbing elements of the cardiac signal, such as noise from the raw ECG data. For noise filtering, a multivariate functional data analysis approach may be used (Pigoli and Sangalli. Computational Statistics and Data Analysis, Vol. 56, 2012, pp 1482-1498). As the signal sensed by sensing device 13 may vary due to a patient's movements, the baseline frequency of raw ECG data 52 may be removed by pre-processor 38 and the cardiac signal may be expressed at a chosen frequency. The frequencies of the signal corresponding to the patient's movements may be removed using median filtering (Kaur et al., Proceedings published by International Journal of Computer Applications, 2011, pp 30-36). Applying raw ECG data 52 to pre-processor 38 generates pre-processed ECG data 55. At this point, ECG platform 37 may cause pre-processed ECG data 55 to optionally be communicated to ECG application 29 running on system device 14 for display on display 17. ECG platform 37 may alternatively, or additionally, cause pre-processed ECG data 55 to be used as an input at classification step 58, discussed in more detail.

At step 56, ECG platform 37 causes pre-processed ECG data 55 to be applied to delineator 39 for delineation. Delineator 39 applies a first neural network that is a delineation neural network to pre-processed ECG data 55. A neural network refers to a mathematical structure or algorithm that may take an object (e.g., matrix or vector) as input and produce another object as an output though a set of linear and non-linear operations called layers. For example, the input of the first neural network may be one or more multi-lead cardiac signals that are pre-processed to remove noise and/or baseline wandering.

To apply pre-processed ECG data 55 to the first neural network, delineator 39 may cause some or all of raw ECG data 52 to be expressed as matrix X, which may be a matrix of real numbers. For example, matrix X may be a matrix of size m×n at the frequency used for training the networks, described in more detail below. The constant “m” may be a number of leads in sensing device 13, which is typically 12, though any number of leads may be used. In this example, the number of samples “n” provides the duration of the cardiac signal “n/f” with f being the sampling frequency of the cardiac signal. The sample rate is above a predetermined rate and is preferably relatively high, such as, for example, at least 20, at least 250, at least 500 or at least 1000 samples per second, etc. In one embodiment, all of the sampled ECG data is transferred to the server for input into the processing algorithms without filtering out ECG data. While the ECG data applied to the first neural network is preferably pre-processed ECG data 55, it is understood that a non-preprocessed cardiac signal (i.e., raw ECG data 52, or a portion thereof) may be applied to the first neural network.

The first neural network may provide as an output, values corresponding to the likelihood of the presence of or one or more waves at a plurality of time points in the cardiac signal. The time points may be dictated by the raw ECG data, may be selected by the user of system device 14, or may be preprogrammed. The first neural network may be a convolutional neural network, and is preferably a fully convolutional neural network. Convolutional neural networks are a particular type of neural network where one or more matrices, which are learned, do not encode a full linear combination of the input elements, but the same local linear combination at all the elements of a structured signal, such as a cardiac signal, through a convolution (Fukushima, Biol. Cybernetics, Vol. 36, 1980, pp 193-202, LeCun et al., Neural Computation, Vol. 1, 1989, pp 541-551). A network which only contains convolutional networks is called a fully convolutional neural network.

Accordingly, at step 56, delineator 39 causes the first neural network to read each time point of the cardiac signal, spatio-temporally analyze each time point of the cardiac signal, and assign a score at each time point corresponding to one or more types of waves. In this manner, all types of waves in the cardiac signals may analyzed and the likelihood of their presence at each time point, quantified, in a single step. Accordingly, each score generated by delineator 39 is indicative of the likelihood of the presence of a particular wave type at a given time point of the cardiac signal. The wave types may be any well know wave type such as, P-waves, Q-wave, R-wave, S-wave, Q-waves, R-waves, S-waves, QRS complexes, and/or T-waves, for example. In this manner, delineator 39 may process data sampled multiple times per heart beat across a plurality of heart beats.

The output of the first neural network may be a matrix Y, which may be a matrix of real numbers. For example, matrix Y may be a matrix of the size p×n. Matrix Y may include scores for each type of wave at each time point of the cardiac signal. In matrix Y, “n” is the number of samples, as discussed above with respect to Matrix X, and “p” is the number of wave types plus the number of wave characterizations. As explained in more detail below, wave characterization may correspond to conductivity, prematurity, ectopy, and/or origin of the waves in the cardiac signal, for example. In one example, the wave types include (1) P-waves, (2) QRS complexes, and (3) T-waves, and the wave characterizations include (1) premature waves, (2) paced waves, (3) ventricular QRS complexes, (4) junctional QRS complexes, (5) ectopic P waves, and (6) non-conducted P waves. Accordingly, in this example, p=3+6=9. Each wave type may be expressed according to certain characteristics of that wave, such as start and end points (i.e., onset and offset)).

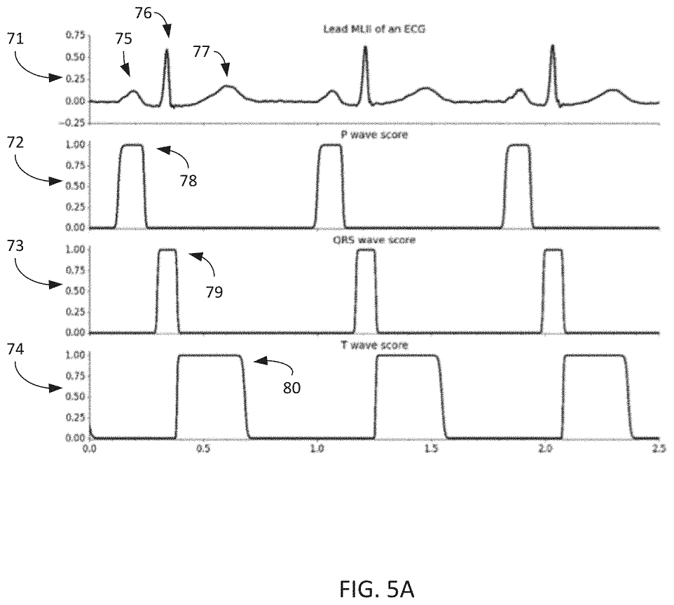

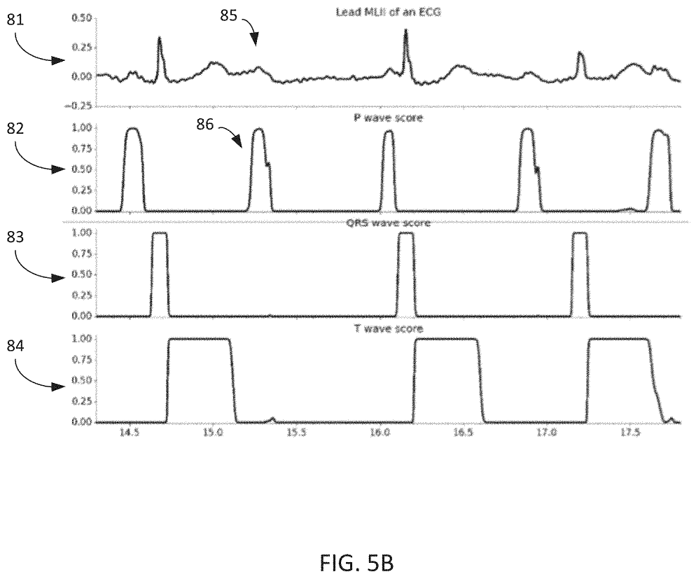

Referring now to FIGS. 5A and 5B, exemplary outputs of the first neural network are graphed for each wave type to illustrate the value of generating scores at each time point corresponding to a plurality of types of waves. Specifically, FIG. 5A illustrates an exemplary output where the delineation neural network processed a normal cardiac signal (with no abnormalities) and FIG. 5B illustrates an exemplary output where the delineation neural network processed a cardiac signal having “hidden” P-waves, for example due to an atrioventricular block.

Referring now to FIG. 5A, four line graphs are illustrated, each graph showing time on the x-axis. Line graph 71 represents the cardiac signal over multiple beats. The plotted signal reflects the well-known ECG waveform having a P-Wave (point 75), QRS complex (point 76), and T-wave (point 77). Line graph 72 is a graph the P-wave score over the same time points in the cardiac signal. Similarly, line graph 73 and line graph 74 are graphs of the QRS score and T-wave scores, respectively, over the same time points. The y-axis for each line graphs 72-74 is the score assigned at each time point, ranging from 0 to 1, with 0 indicating a low likelihood of the presence of a particular wave and 1 indicating a high likelihood of the presence of a particular wave. For example, line graph 72 indicates a very high likelihood of the presence of P-waves at score 78 which corresponds to the time points near point 75, line graph 73 indicates a very high likelihood of the presence of a QRS complex at score 79 which corresponds to the time points near point 76, and line graph 74 indicates a very high likelihood of the presence of a T-wave at score 80 which corresponds to the time points near point 77.

FIG. 5B, like FIG. 5A, illustrates four line graphs, line graphs 81-82, which are similar to line graphs 71-74. Specifically, line graph 81 represents the cardiac signal over several beats, line graph 82 represents the P-wave score over the cardiac signal, line graph 83 represents the QRS score over the cardiac signal, and line graph 84 illustrates the T-wave score over the cardiac signal. Unlike FIG. 5A, the ECG signal in line graph 81 includes hidden P-waves such as the hidden P-wave shown at point 85. Hidden P-waves are P-waves that occur during another wave or complex such as a T-wave. As the cardiac signal processed by the delineation network involves a high sample rate and the delineation network generates data for each wave type at each time point, the output recovered is robust enough (i.e., includes enough sample points) to identify two waves occurring at the same time, such as the case with hidden P-waves. For example, line graph 82 indicates a very high likelihood of the presence of P-waves at score 86 which corresponds to the time points near point 85. Accordingly, it is understood that the delineation neural network is not limited to recovering only one wave at each time point and therefore can identify several waves at any time point. It is further understood that signals from one or more leads may be processed simultaneously by the first neural network.

Using the scores assigned to each time point corresponding to each wave type (e.g., P-wave, QRS complex, T-wave, etc.), delineator 39 may post-process the cardiac signal. Post-processing involves, assigning to each time point, none, one, or several waves, calculating the onset and offset of each of the identified waves, and optionally determining the characterization of the waves. Waves may be assigned to each time point by determining that a wave exists at that time point if a certain value is achieved. Computing the “onset” and “offset” of each wave involves computing the time points of the beginning and the end of each wave in the cardiac signal, the beginning referred to as the “onset” and the end referred to as the “offset.” This may involve analyzing the time points corresponding begging and end of the highest values for each wave type. Delineator 39 may characterize the waves by identifying prematurity, conductivity and ectopy. Wave characterization leverages the contextual information between each wave and/or each beat. For example, the premature label may be applied to the wave if a certain threshold value is achieved at a certain time point or an average value over several time points.

After computing the onset and offset of each wave type in the cardiac signal, delineator 39 may calculate global measurements. Global measurements are derived from the onset and offset of each wave type and may relate to features and characteristics of the cardiac signal such as intervals between waves and wave durations. For example, global measurements may include, but are not limited to, PR interval, P-wave duration, QRS complex duration, QRS axis, QT interval, corrected QT interval (Qtc), T-wave duration, JT interval, corrected JT interval, heart rate, ST elevation, Sokolov index, number of premature ventricular complexes, number of premature atrial complexes (PAC), ratio of non-conducted P waves, and/or ratio of paced waves.

Delineator 39 may further deduce labels solely from the information generated by delineator 39. For example, the following labels may be deduced by delineator 39: short PR interval (i.e., PR interval<120 ms), first degree AV block (e.g., PR interval>200 ms), axis deviations, long QTc, short QTc, wide complex tachycardia, and/or intraventricular conduction blocks. Labels determined solely from information generated by delineator 39 are referred to as delineation based labels.

Referring again to FIG. 4, ECG platform 37 may cause the output of step 56 (e.g., wave information 62) as well as pre-processed ECG data 55 to be communicated or otherwise applied to clusterer 42 for clustering at step 63. Wave information 62 may include scores regarding PVC waves and PAC waves including onsets and offsets generated and relevant durations. Clusterer 42 may process wave information 62 and identify clusters of PAC or PAV waves during the duration of the cardiac signal. Once identified, clusterer 42 may assign cluster label 64 to one or more time windows, identifying either a PVC or a PAC cluster for each time window. A time window is defined by two time points in the cardiac signal.

Referring again to FIG. 4, ECG platform 37 may also cause the output of step 56 (e.g., wave information 57) as well as pre-processed ECG data 55 to be communicated or otherwise applied to classifier 41 for classification at step 58. Classification at step 58 involves applying a second neural network (i.e., classification neural network) to pre-processed ECG data 55. Accordingly, in one example, the input of the second neural network may be one or more multi-lead cardiac signals with variable length that is pre-processed. Classifier 41 may also process wave information 57 and/or other information such as patient-specific information including the patient's age or any relevant clinical information. As explained above, ECG platform 37 may cause optionally cause pre-processed ECG data 55 to be communicated directly to classifier 41 and processed by classifier 41 if delineation at step 56 is not necessary. In this manner, classifier 41 may process data sampled multiple times per heart beat across a plurality of heart beats.

The second neural network generates an output having values that correspond to the likelihood of the presence of one or more abnormality, condition and/or descriptor at each time point of the cardiac signal. If a time point or time window is determined to correspond to a certain abnormality, condition, and/or descriptor, a label corresponding to that abnormality, condition, and/or descriptor will be assigned to that time point or window. In one example, one or more labels 59 may be assigned to a time point or time window if a score achieves a predetermined threshold. Accordingly, multi-label localization may be achieved for abnormalities, conditions, and/or descriptors by generating a plurality of values at each time point and assigning one or more labels at each time point.

Classifier 41 may recover the output of the classification neural network as a vector of size q. The values in the vector correspond to the presence of each label at each time point or each time window. For example, the output of the classification neural network may be the vector [0.98:0.89; 0.00] with the corresponding labels for each element of the vector: Right Bundle Branch Bloc; Atrial Fibrillation; Normal ECG. The scores may be between 0 and 1. For the vector above, a threshold of 0.5 would result in the labels “Right Bundle Branch Block” and “Atrial Fibrillation” being assigned by classifier 41 to the time point or time window corresponding to the score. It is understood that the threshold may be preprogrammed and/or selected by the user and may be modified to provide varying degrees of sensitivity and specificity. By assigning one or more labels for each time point, onsets and offsets corresponding to each label may be computed to identify durations of episodes (e.g., abnormalities episodes).

Abnormalities and conditions may include any physiological abnormality or condition which may be identifiable on the cardiac signal. Today about 150 measurable abnormalities may be identified on cardiac signal recordings. Abnormalities and conditions may include but are not limited to, sinoatrial block, paralysis or arrest, atrial fibrillation, atrial flutter, atrial tachycardia, junctional tachycardia, supraventricular tachycardia, sinus tachycardia, ventricular tachycardia, pacemaker, premature ventricular complex, premature atrial complex, first degree atrio-ventricular block (AVB), 2nd degree AVB Mobitz I, 2nd degree AVB Mobitz II, 3rd degree AVB, Wolff-Parkinson-White syndrome, left bundle branch block, right bundle branch block, intraventricular conduction delay, left ventricular hypertrophy, right ventricular hypertrophy, acute myocardial infarction, old myocardial infarction, ischemia, hyperkalemia, hypokalemia, brugada, and/or long QTc. Descriptors may include descriptive qualities of the cardiac signal such as “normal” or “noisy ECG.”

Upon applying the second neural network at step 58, classifier 41 may read each time point of the cardiac signal as well as each global measurement, analyze each time point of the cardiac signal and each global measurement, compute time windows by aggregating at least two time points, and compute scores for each time window, the scores corresponding to a plurality of non-exclusive labels.

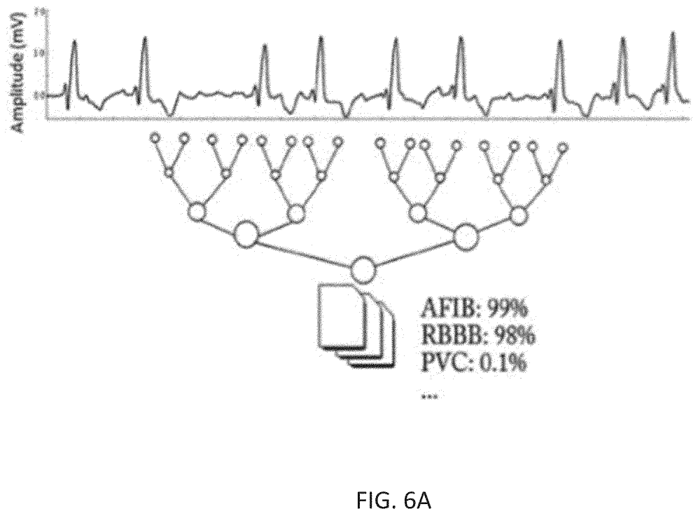

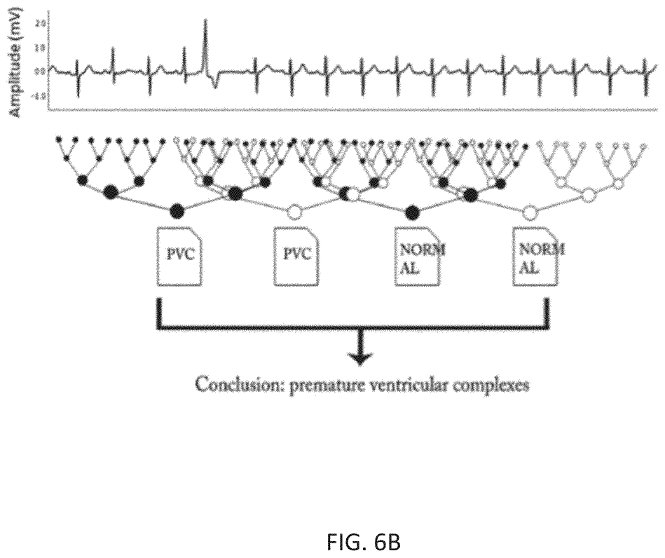

The classification neural network may be a convolutional neural network or a recurrent neural network. Referring now to FIG. 6A, a classification neural network in the form of a convolutional neural network is illustrated applied to an ECG signal. Most convolutional neural networks implement a few convolutional layers and then standard layers so as to provide a classification. The ECG signal is given as input to the network, which aggregates the information locally and then combines it layer by layer to produce a high-level multi-label classification of the ECG. For each label a score is provided. The labels of the convolutional neutral network shown in FIG. 6A include atrial fibrillation (AFIB), right bundle branch block (RBBB) and, and premature ventricular complex (PVC).

Referring now to FIG. 6B, a classification neural network in the form of a recurrent convolutional neural network is illustrated. Similar to FIG. 6A, the ECG signal is given as input to the network. A recurrent convolutional neural network refers to a particular convolutional neural network structure able to keep memory of the previous objects it has been applied to. A recurrent convolutional neural network is composed of two sub-networks: a convolutional neural network which extracts features and is computed at all time points of the cardiac signal, and a neural network on top of it which accumulates through time the outputs of the convolutional neural network in order to provide a refined output. In this manner, the convolutional neural network acts as a pattern detector whose output will be accumulated in time by the recurrent neural network.

As is shown in FIG. 6B, the output of the convolutional neural network identified four labels at various time points including premature ventricular complex (PVC) and Normal. Those labels were then applied to the second neural network which produced the refined output “premature ventricular complex.” In this example, the network correctly recognized a premature ventricular complex (PVC, the fifth and largest beat) in the first part of the signal while the second part of the signal is considered normal. As the cardiac signal includes abnormality, it cannot therefore be considered as normal, and the accumulated output is therefore PVC.

The first neural network (i.e., delineation neural network) and the second neural network (i.e., classification neural network) must be trained to achieve the behavior and functionality described herein. In both the delineation and the classification embodiments, the networks may be expressed using open software such as, for example, Tensorflow, Theano, Caffe or Torch. These tools provide functions for computing the output(s) of the networks and for updating their parameters through gradient descent.

Training the neural networks involves applying numerous datasets containing cardiac signals and known outputs to the neural networks. A database of the datasets containing cardiac signals collected across a plurality of patients using the systems and methods described herein may be stored on server 15 and/or drive 16 (e.g., in the cloud). The datasets in the database may be used by server 15 to analyze new cardiac signals inputted into the system for processing. In a preferred embodiment, any cardiac signal applied to the trained neural network will have the same sampling rate and/or frequency as the cardiac signals in the datasets used to train the neural network. For example, training of the classification neural network begins with a dataset containing cardiac signals and their known delineation. As explained above, the cardiac signal is expressed as a matrix of size m×n at a predefined frequency. For example, the network may be trained at 250 Hz, 500 Hz or 1000 Hz, though any frequency could be used. The delineation is then expressed in the form of a Matrix Y of size p×n where p is the number of types of waves. Each wave is expressed with their start and end points such as, for example: (P, 1.2 s, 1.3 s), (QRS 1.4 s 1.7 s), (T, 1.7 s, 2.1 s), (P, 2.2 s, 2.3 s). In this example, the first row of Matrix Y corresponds to P-waves, and will have a value of 1 at times 1.2 s and 1.3 s, and as well as 2.2 s and 2.4 s, and 0 otherwise. The second row of Matrix Y corresponds to QRS complexes and will have a value of 1 at times 1.4 s and 1.7 s, and otherwise 0. Finally, the third row of Matrix Y corresponds to T-waves and will have a value of 1 at times 2.2 s and 2.3 s, and otherwise 0. The parameters of the network may then be modified so as to decrease a cost function comparing the known delineation and the output of the network. A cross-entropy error function is used so as to allow for multi-labeling (i.e., allowing for multiple waves at a given instant). This minimization can be done though a gradient step, repeating the foregoing steps at least once for each cardiac signal of the dataset. It is understood that a similar approach may be used to train the delineation neural network (i.e., second neural network).

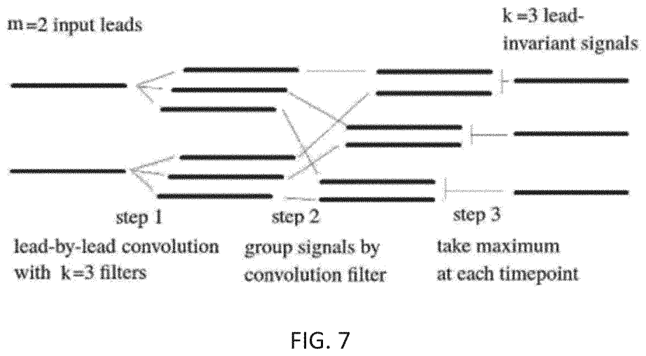

It is further understood that ECG platform 37 may cause neural networks described herein to process cardiac signals having a differing number of leads in entry. For example, the neural network may include a sequence of layers at the beginning of the network so as to obtain a network which is independent of the number of input leads and can therefore process cardiac signals with any number of leads m. For example, FIG. 7 illustrates two input leads (m=2) and three output signals (k=3). However, the same structure can process any number of input leads m and will still provide the same number of output signals, which can be fed to the rest of the network for which a fixed number of input signals is required. For this reason, the number of input leads may vary and need not be fixed.

As is shown in FIG. 7, to obtain k signals from an m input leads, the leads may be convoluted using a lead-by-lead convolution with k filters. The signal may then be grouped by a convolution filter in order to obtain k groups of m leads and a mathematical function is finally applied to each group to obtain k leads. The mathematical function may be the maximum at each time point or may be any other function known to one skilled in the art.

Referring again to FIG. 4, at step 61, ECG platform 37 may cause labels for each time window (i.e., labels) to be aggregated by post-processor 43 to generate processed labels 60. The labels may be derived from global measurements based on delineation. For example, the label corresponding to first degree atrioventricular block may be derived from a PR interval longer than 200 ms. As explained above, the PR interval is a global measurement based on the delineation. Post-processor 43 may also aggregate the delineation-based labels with the classification labels corresponding to the same time points.

Post-processor 43 may also filter the labels to remove redundant labels, assemble labels according to a known hierarchy of labels, or ignore labels that are known to be of lesser importance according to a hierarchy or weighted values. Post-processor 43 may also aggregate the labels through time so as to compute the start (onset) and end (offset) times of each abnormality. It is understood that post-processor 43 may be a standalone component or may be a subcomponent of classifier 41.

As is shown in FIG. 4, the information generated on back end 46 by ECG platform 37 in steps 54, 56, 58 and 61, and optionally, 63, may be communicated by ECG platform 37 to ECG application 29 on front end 45. ECG application 29 may cause the foregoing information to be displayed, at step 65, on display 17 of system device 14. The information generated on back end 46 may be automatically transmitted by ECG platform 37 or ECG platform 37 may cause the information to be stored on server 15 until requested by ECG application 29. Upon generating the data, ECG platform 37 may transmit a message to ECG application 29, informing ECG application 29 that the data is available from ECG platform 37.

ECG application 29 may receive data (e.g., raw ECG data, pre-processed ECG data, wave information, labels and any other data generated during steps 54, 56, 58, 61, and/or 63) and cause system device 14 to display as described in U.S. Patent Pub. No. 2020/0022604, the entire contents of which are incorporated herein by reference. Specifically, the '604 publication explains that the ECG signal, features of the ECG signal, and/or descriptors of the ECG signal may be displayed in a multiple field display in an interactive manner.

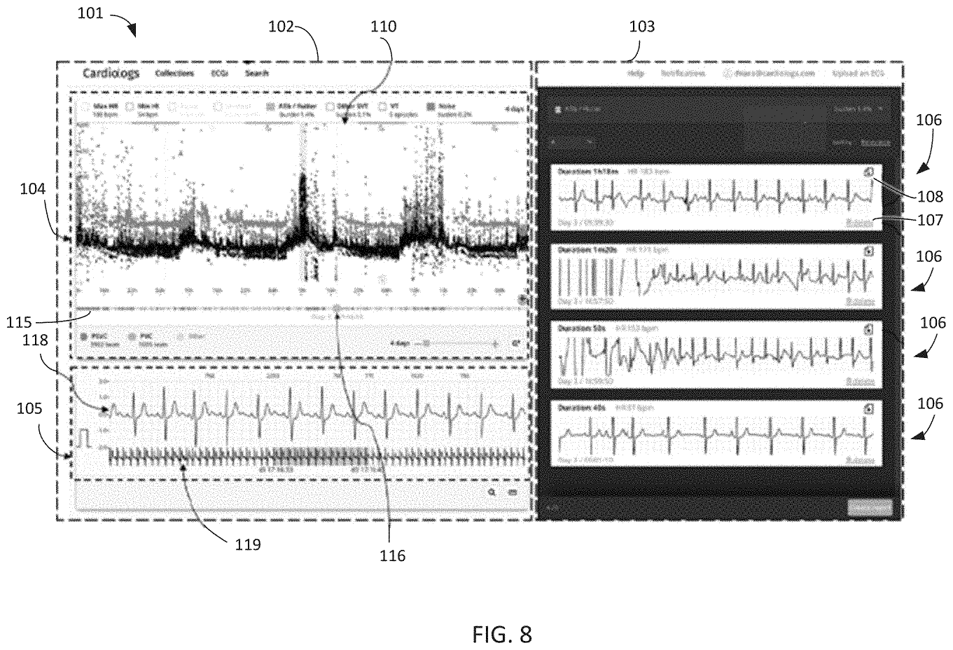

Referring now to FIG. 8, an exemplary display, interactive display 101, is illustrated. Interactive display 101 includes first side 102 and second side 103. First side 102 further includes second graphic window 105 and first graphic window 104, having plot 110 which includes data corresponding to the ECG signal. First graphic window 104 includes plot 110 providing a global view of an ECG signal.

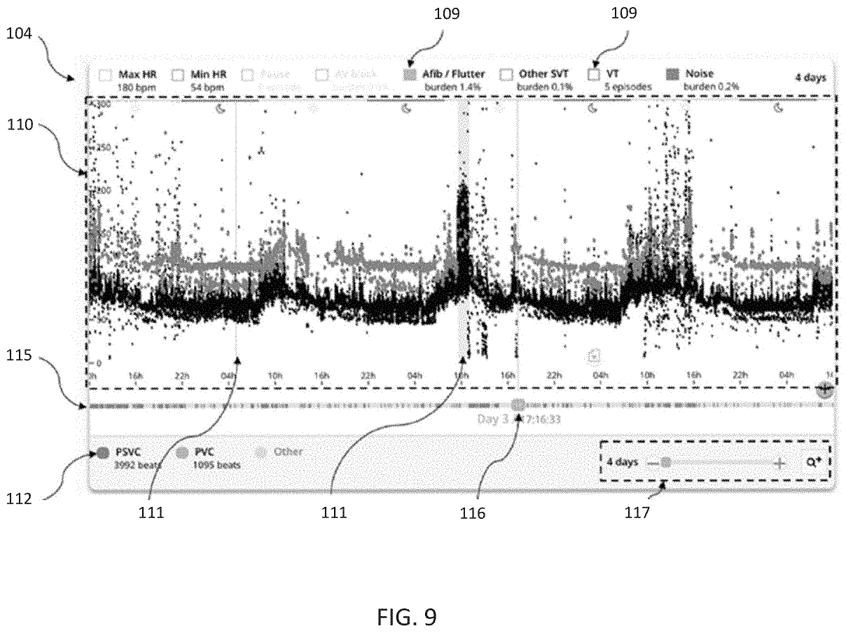

Referring now to FIG. 9, a zoomed-in version of first graphic window 104 is illustrated. In this exemplary display, plot 110 is an heart rate density plot (HRDP) which represents R-R intervals (interval between two QRS waves) through time. As shown in FIG. 9, the upper region of first graphic window 104 comprises multiple label buttons 109. Each label button 109 has, displayed in its proximity, text describing the label to which it is associated. Each label button 109 is associated with a color so that, when label button 109 is selected by the user, graphic portion 111 is displayed on the plot 110 to visually indicate the presence the episodes and/or events corresponding to the label associate with label button 109. This provides visual references for the user permitting easy identification of a specific category of events and/or episodes along the cardiac signal. In the exemplary display illustrated in FIG. 9, secondary labels 112 are included. In this exemplary display, secondary labels 112 include beat label PVC (premature ventricular complex) and PSVC (premature supraventricular complex), though it is understood that other secondary labels may be included. The points in the plot 110 associated with the label PVC and PSVC are colored, as shown in FIG. 9 by the presence of points of color different from black.

First graphic window 104 further comprises, parallel to the time axis of the plot 110, temporal bar 115. Temporal bar 115 provides a linear representation of the total ECG acquisition time wherein the time periods associated to episodes or events are represented as colored segments. As is shown in FIG. 9, the darker grey zones on temporal bar 115 correspond to time periods of noisy signal (e.g., when the signal is too artifacted and the analysis algorithm cannot propose a delineation and proper detection). First graphic window 104 further comprises interactive cursor 116. A user using ECG application 29 may move interactive cursor 116 along temporal bar 115 to allow a navigation of the plot 110 along the total ECG acquisition time. In the right bottom corner of first graphic window 104, first graphic window 104 comprises second interactive means 117 configured to cause plot 110 to zoom in and out.

Referring again to FIG. 8, second side 103 includes multiple episode plots 106. Each episode plot 106 displays at least one segment of the ECG strip corresponding to a detected episode and may include text regarding the duration (e.g., “Duration: 1 h38 m”) and/or the starting time of the episode (e.g., “Day 3/09:39:30”). Each episode plot 106 includes third interactive icon 108 to select the corresponding episode plot for inclusion in a report. Each episode plot 106 further includes fourth interactive icon 107 which permits the user to remove the respective ECG plot from interactive display 101. Second side 103 may further include text describing one or more of episode plots 106.

Interactive display 101 further includes graphic window 105 including ECG strip 118 in a second time window starting at the time point selected by the cursor 116. Second graphic window 105 further includes ECG strip 119 in a third time window which is larger than the second time window which is inclusive of the second time window. The third time window includes a shaded portion which corresponds to the second time window.

Referring now to FIG. 10, a similar display, interactive display 121, is illustrated. Interactive display 121 includes first side 122 and second side 123. First side 122 further includes first graphic window 124 and second graphic window 125. Second side 113 has the same functionality as second side 103 described above, and includes episode plots 126 similar to episode plots 106. Further, second graphic window 125 has the same functionality as second graphic window 105, and includes ECG strip 138 and ECG strip 139 similar to ECG strip 118 and ECG strip 119.

First graphic window 124 is similar to first graphic window 104 except for plot 130. Like first graphic window 104, first graphic window 124 includes multiple label buttons 129 having the same functionality as multiple label buttons 109, secondary labels 132 having the same functionality as secondary labels 112, temporal bar 135 and curser 136 having the same functionality as temporal bar 115 and cursor 116, and second interactive means 137 having the same functionality as second interactive means 117. Unlike plot 110, plot 130 is a heart rate density plot which is the projection onto a bivariate intensity plot of the histogram of the density of heart rates as a function of time.

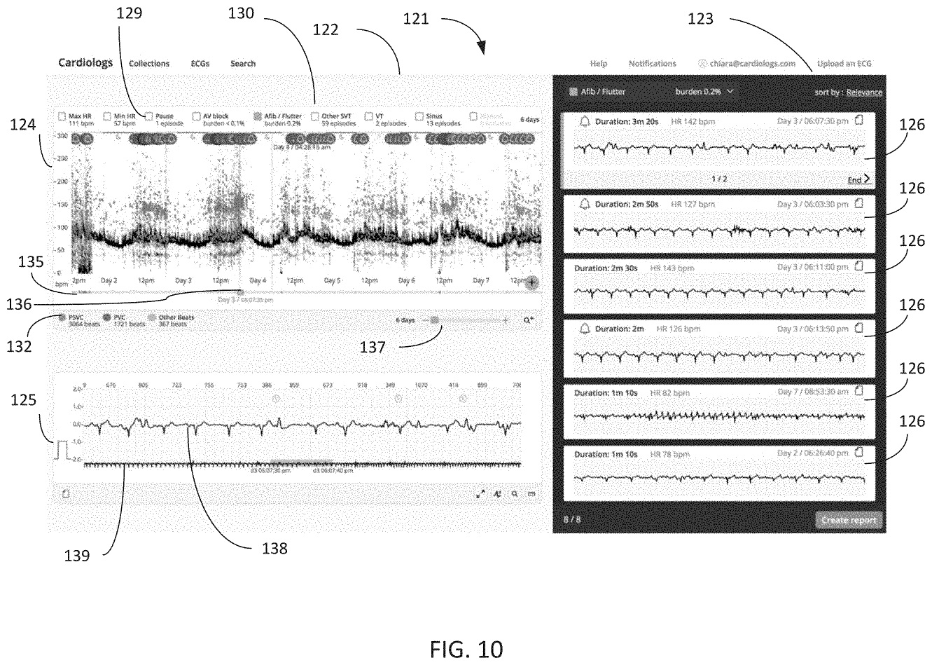

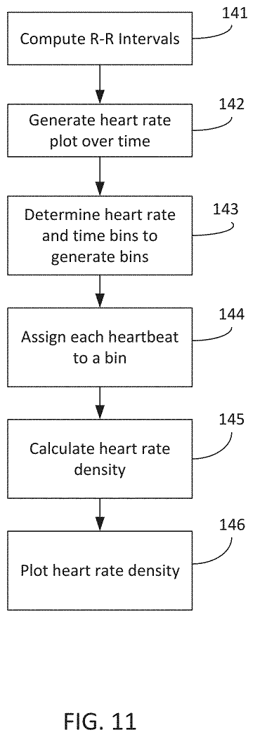

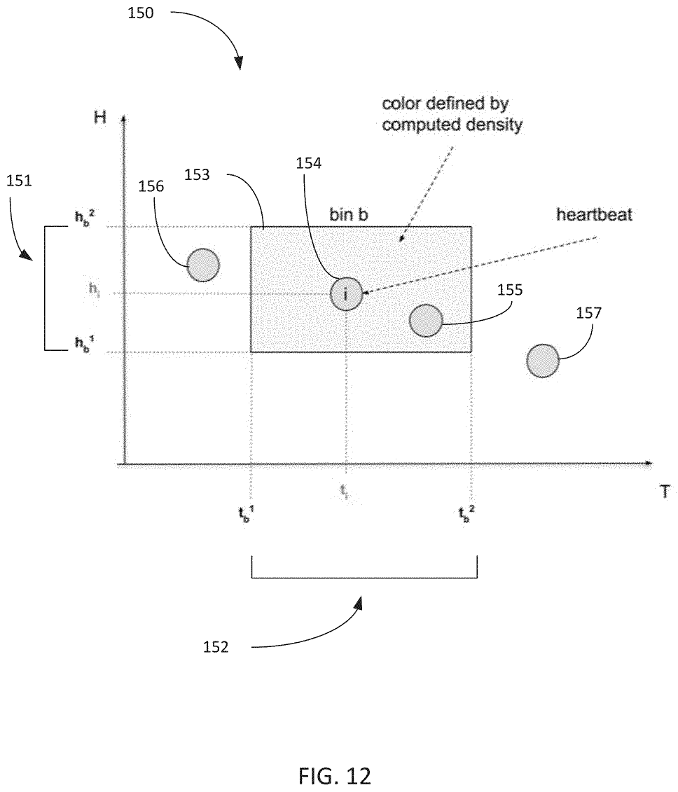

Referring now to FIG. 11, steps for generating and plotting a heart rate density plot, such as plot 130, are provided. At step 141, ECG platform 37 computes R-R intervals in the cardiac signal (i.e., ECG data). For example, ECG platform 37 may apply the cardiac signal to the delineation neural network to determine the RR intervals, as described above. At step 142, ECG platform 37 may generate the heart rate plot over time. An exemplary heart rate plot, HRDP 150, is illustrated in FIG. 12.

As is shown in FIG. 12, time is projected along the x-axis and the heart rate (e.g., beats per minute) is projected along the y-axis. In one embodiment, both time and heart rate are scaled linearly. However, time and/or heart rate may be scaled logarithmically or using other well-known scales. For simplicity, only four heart beats are shown in FIG. 12.

Referring again to FIG. 11, at step 143, ECG platform 37 may cause the y-axis and the x-axis may be divided into elementary elements, referred to as HR bins and time bins respectively. For example, in FIG. 12, HR bin 151 and time bin 152 are illustrated. HR bin 151 is defined by a first and second heart rate value (e.g., h_{b}¹ and h_{b}²). Similarly, time bin 152 is defined by a first and second time value (e.g., t_{b}¹ and t_{b}²). The intersection of a HR bin and a time-bin will be referred to as a bin. In other words, a bin will be defined by a first and second heart rate value and a first and second time value. In FIG. 12, bin 153 is illustrated and defined by HR bin 151 and time bin 152.

Referring again to FIG. 11, at step 144, ECG platform 37 will cause each heartbeat to be assigned to a bin. Specifically, a heartbeat (e.g., QRS complex) that occurs during a time window of a given time bin is included in the computation of the column corresponding to that time bin. Further, a heart rate corresponding to that heartbeat determines which HR bin it belongs to in the column defined by the time bin. For example, in FIG. 12, heartbeat 154 and heartbeat 155 each have a corresponding time and heart rate value that fall within time bin 152 and HR bin 151, respectively. Conversely, heartbeat 156 and heartbeat 157 each have a time value that falls outside time bin 151 and thus neither are included in bin 153.

Referring again to FIG. 11, at step 145, ECG platform 47 will calculate the heart rate density for each time bin. For a given bin, the area defined by the respective time bin and heart rate bin will be represented according to the density of the heart beats comprised in the bin (i.e., number of heartbeats within the bin). Each bin may then be color coded according to the density. For example, each bin may have certain shades of colors or patterns, such as grey levels, for example. In the example in FIG. 12, bins may be represented as levels of grey that get darker as the density of heart rates increases. As is shown in FIG. 12, bin 153, which includes 2 heartbeats, may be represented by a darker shade of grey than a bin with only 1 heartbeat, but a lighter shade of grey than a bin having 3 or more heartbeats.

In a preferred embodiment, the density is calculated as a function of the number of R-waves in the bin divided by the heart rate of the HR bin (e.g. the mean of the minimum and maximum bounds of the time window). This preferred computation of density considers the time spent in a specific bin. For example, in a time bin of 3 minutes, if there occurs 100 beats at a heart rate of 50 bpm (beats per minute) in a first HR bin and 100 beats at 100 bpm in a second HR bin, there will be as many beats in each bin, but 2 minutes will be spent at 50 bpm and only one minute at 100 bpm. Therefore, this bin would have the same density representation if only the number of beats are considered. However, when considering the count of beats divided by the heart rate, the first bin corresponding to the heart rate bin of 50 bpm will be darker than the bin corresponding to the heart rate bin of 100 bpm, as dividing by the heart rate gives higher weight to lower heart rate values. The preferred embodiment therefore captures this temporal information better than only considering the count of beats.

Referring again to FIG. 11, at step 146, ECG platform 37 will plot the heart rate density for each bin. It is understood that capturing temporal information in the column (time bin), in addition to the temporal information naturally given as function of the x-axis, facilitates expression of the density in a manner superior to other forms of aggregated representations of the ECG signal, such as the HRDP plot in plot 110.

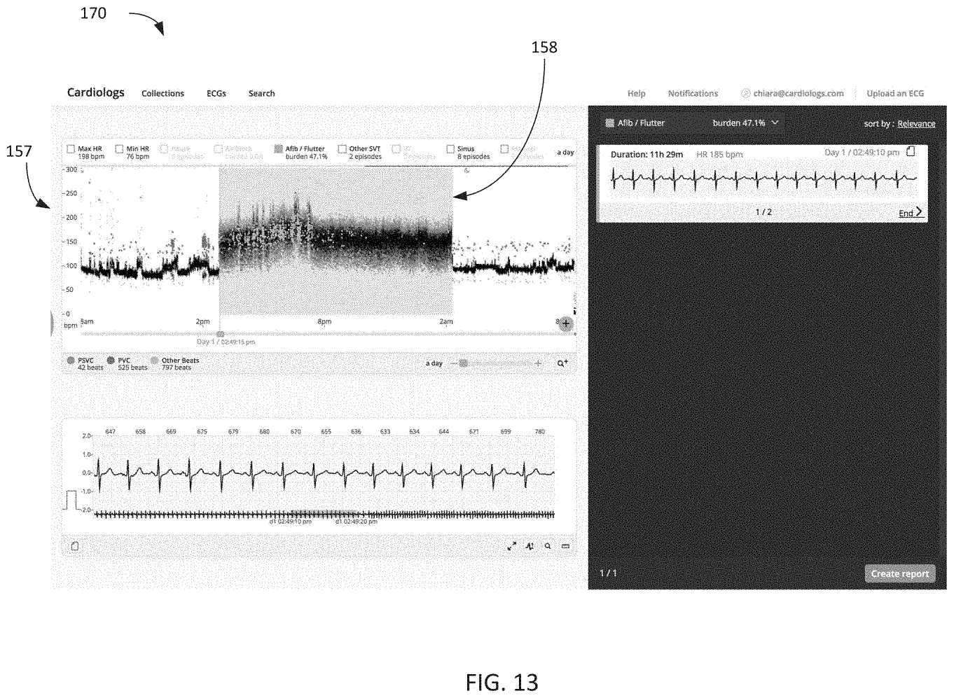

It is understood that the bounds of the x-axis of the HR density plot may be the beginning and end of the signal. However, in a preferred embodiment, the bounds of the x-axis may interactively vary with the action of zooming in and out performed by the user. The bounds of the y-axis remain fixed when performing this action. Referring again to FIG. 10, plot 130 includes interactive means 137 which may be used to zoom-in on the heart rate density plot. The zoom action may only change the size of the plot display. Alternatively, zooming in and out changes the size of the time window corresponding to a time-bin. With the zooming-in action, a bin represented with the same number of pixels covers a shorter time window. Zooming in therefore causes a new computation of the histogram with finer temporal divisions, and consequently, finer temporal information. This allows for a representation of the ECG signal that shows varying levels of aggregation of the information as a function of the time scale one chooses to display, in order for the histogram to remain both readable and informative at any level of zoom. Referring now to FIG. 13, an interactive display, interactive display 170, is illustrated which is similar to the interactive display in FIG. 10. Interactive display 170 has been zoomed-in resulting in plot 159 having zoomed in portion 158.

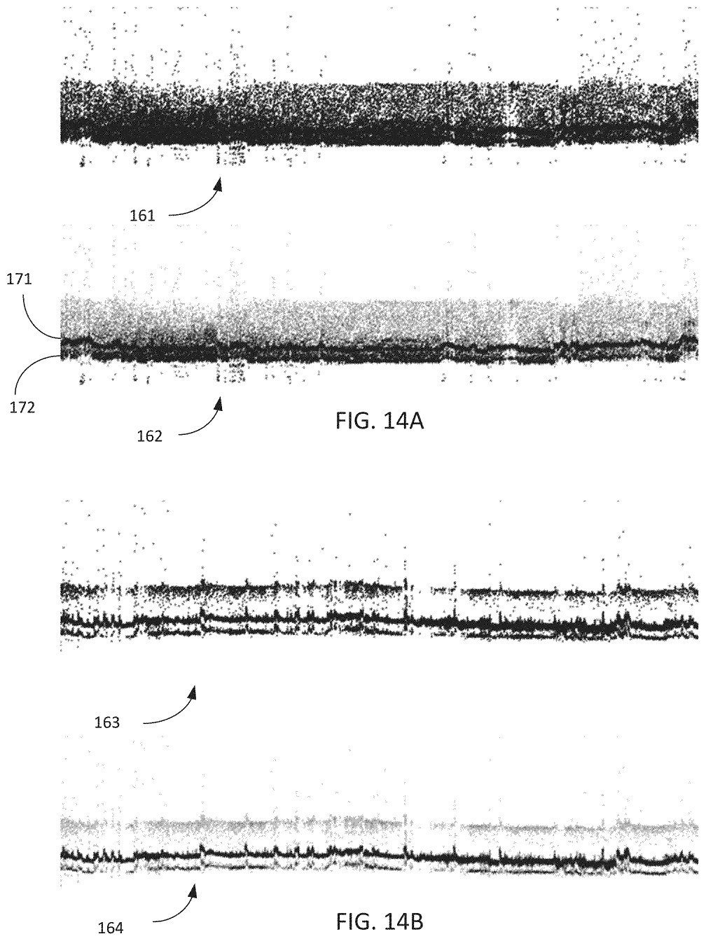

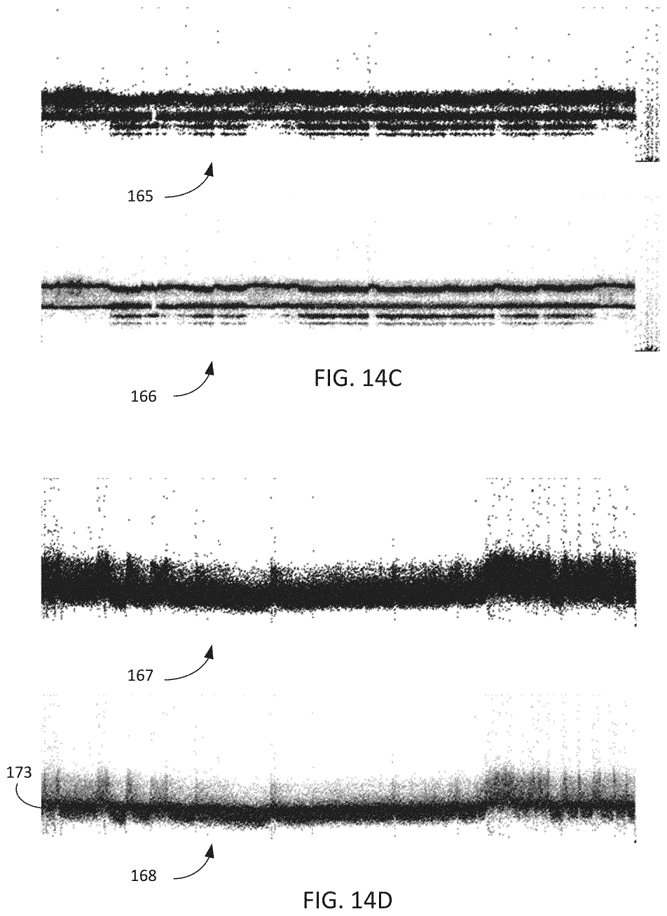

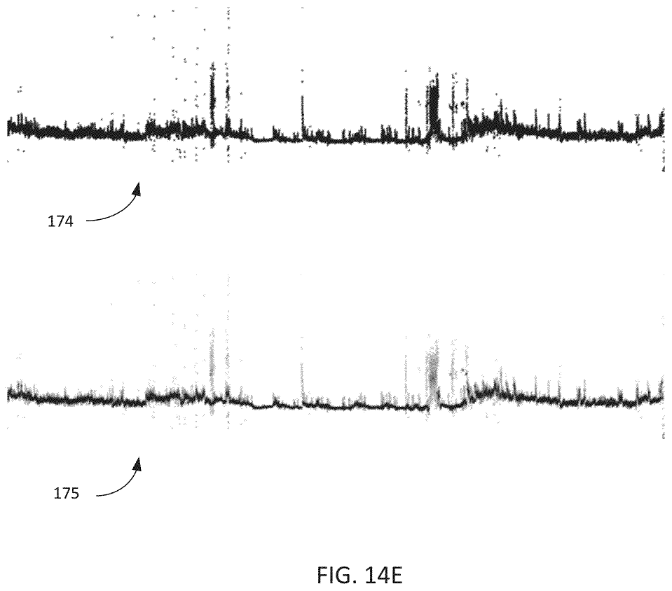

FIGS. 14A-E illustrate the superiority of the HRDP over the typical R-R plot. Referring now to FIG. 14A, a signal generated by a holter having a very high number of PVCs with varying coupling is illustrated as RR plot 161 and density plot 162. In density plot 162, the underlying rhythm is clearly visible as line 171. Further, the compensatory rest is illustrated as line 172 at the bottom. In R-R plot 161, this pattern is less clear. Referring now to FIG. 14B, a signal generated by a holter having less premature complexes than the one in FIG. 14A is illustrated as R-R plot 163 and density plot 164. The main rhythm is clearly illustrated in density plot 164 and is less clear in R-R plot 163. Referring now to FIG. 14C, a signal generated by a holter with vary conduction flutter is illustrated as R-R plot 165 and density plot 166. As is shown in FIG. 14C, the conduction flutter is more emphasized by the four clear black lines in density plot 166 than the four diffuse clouds that appear in the R-R plot 165. Referring now to FIG. 14D, a signal generated by a holter with permanent atrial fibrillation is illustrated as R-R plot 167 and density plot 168. As is shown in this figure, density plot 168 gives more precise information on the variations of the heart rate within the fibrillation. Specifically, darker lower half 173 shows that more time is spent at a low heart rate than at a high heat rate. Density plot 168 further illustrates spikes where the upper half becomes a bit darker corresponding to the heart rate increasing. These nuances are not visible in R-R plot 167. Referring now to FIG. 14E, a signal generated by a holter having paroxysmal atrial fibrillation and otherwise regular rhythm is illustrated as R-R plot 174 and density plot 175. The pattern of a regular rhythm is more visible in density plot 175 where a clear black line emerges. Also, the pattern of atrial fibrillation contrasts more in density plot 175 than R-R plot 174 as the color changes as well (density diminishes which makes the plot lighter).

Referring again to FIG. 4, at step 66, a user using ECG application 29 may interact with an interactive active display described above using input devices 25 to request a report and/or customize the report. A report typically includes portions of the cardiac signal and may involve information pertaining to abnormalities and/or episodes (e.g., episode plots) and/or other information generated during pre-processing (step 54), delineation (step 56), classification (step 58), clustering (step 63) and/or post-processing (step 61). A report may further include patient specific medical data such as the patient's name, age, health history, and/or other medical information. It is understood that any individually identifiable health information, and/or protected health information may be encrypted when communicated between ECG application 29 and ECG platform 37.

As explained above, interactive icons in interactive displays may be engaged to incorporate data and images displayed in a report. For example, third interactive icon 108 may be selected by a user using ECG application 29 to include the corresponding episode plot in a report. Accordingly, at step 66, the user may request a report and may select customized features such as certain data to be included in the report (e.g., abnormality data, episode data, episode plots, etc.).

At step 67, ECG application 29 may transmit the request for a report and selected customizable features (e.g., ECG data to be included in the report) to ECG platform 37 and ECG platform 37 may receive the request and information. ECG platform 37 may log the request and save the information received from ECG application 29. At step 68, ECG platform 37 may cause report generator 44 to generate a report 69 according to the information received from system ECG application 29.

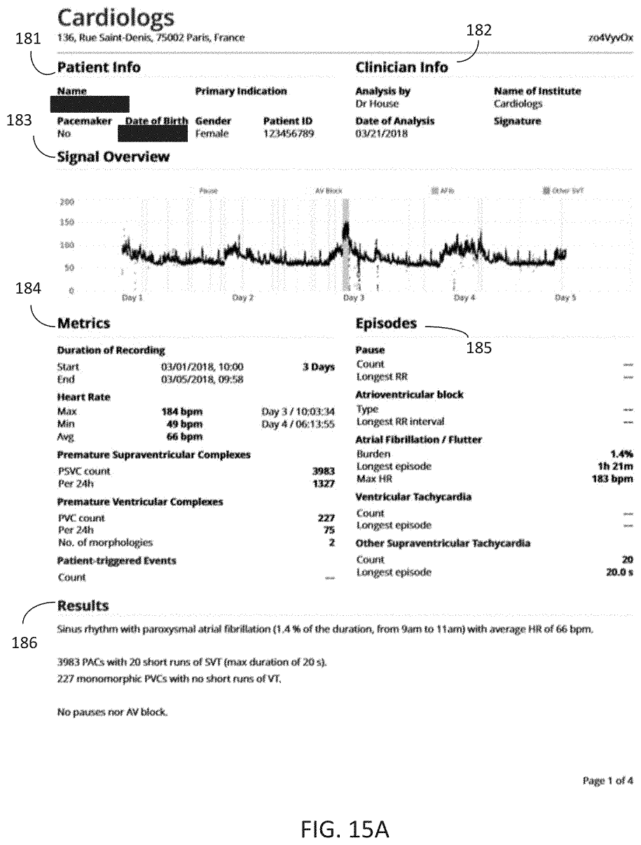

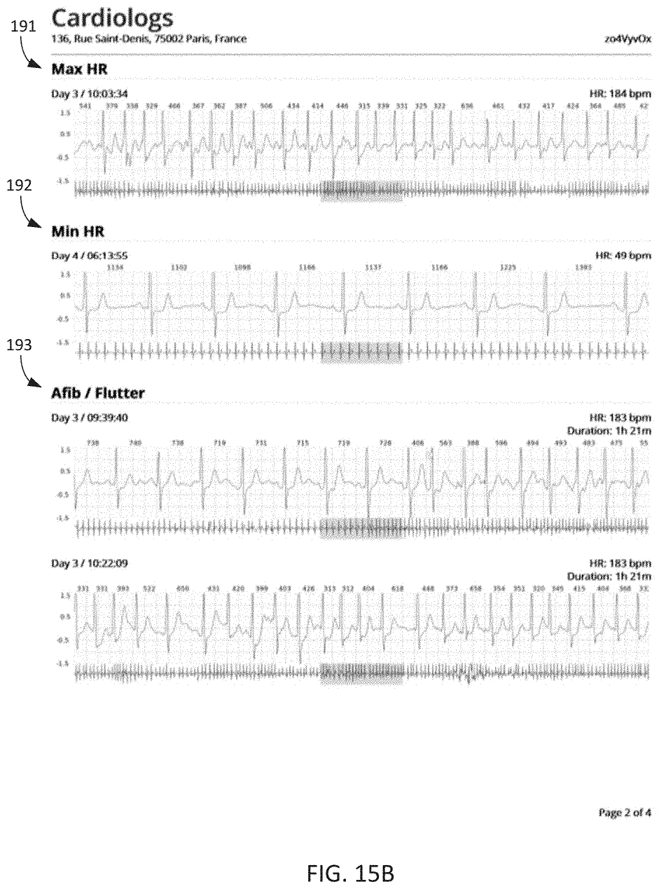

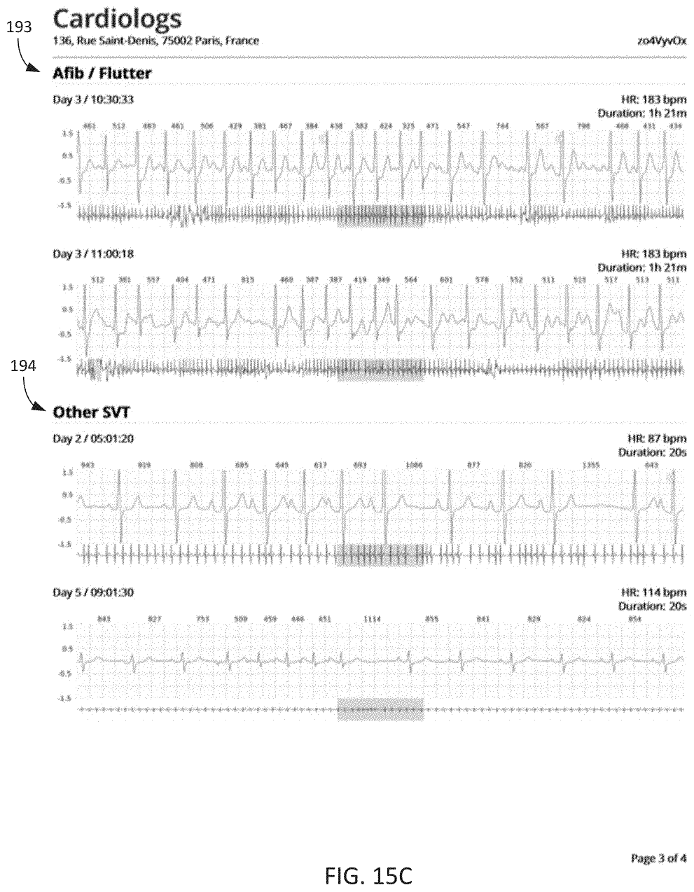

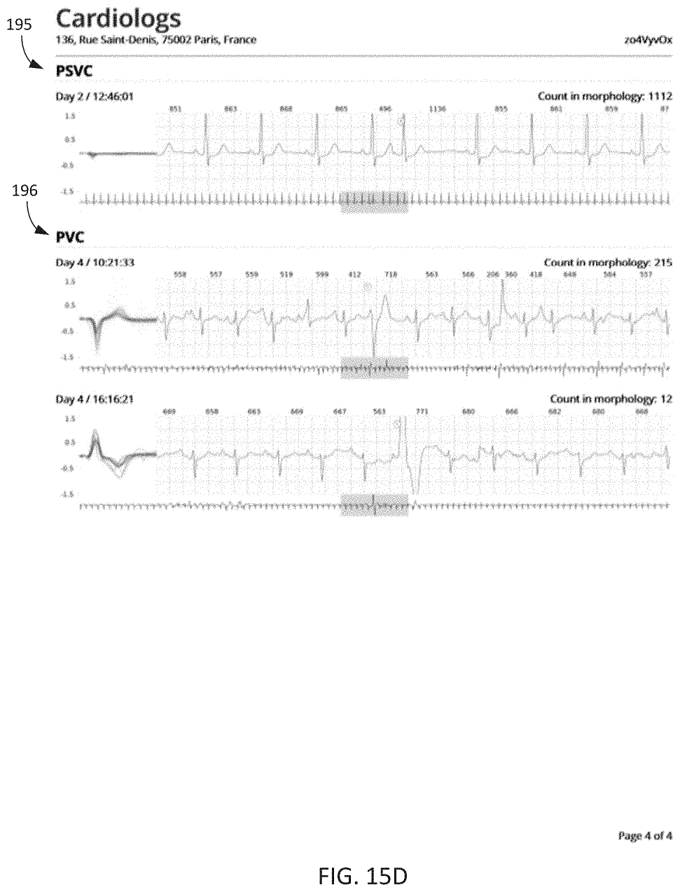

Referring now to FIGS. 15A-15D, an exemplary report generated at step 68 is illustrated. The first page of the exemplary report is illustrated in FIG. 15A. The first page may be presented in several sections such as first section 181, second section 182, third section 183, fourth section 184, fifth section 185, and sixth section 186. First section 181 may include patient specific information such as, for example, the patient's name, primary indication, whether the patient has a pace maker, the patients date of birth, gender and/or a patient ID. Second section 182 may include clinician information such as, for example, the overseeing physician, the name of the institute, the date of the analysis and/or a signature.

Third section 183 may include a plot of the ECG data. In FIG. 15A, section 183 includes a heart rate density plot similar to the one shown in FIG. 12. The window of time shown may be a default time or may be a user defined time window. Like the heart rate density plot in FIG. 12, a certain label may be selected to indicate the occurrence of an abnormality on the density plot. The time window is usually selected according to the relevant episodes and/or events. It is understood, however, that other plots may be included in the report such as an R-R plot.

Fourth section 184 may include metrics from the cardiac signal recording. For example, fourth section 184 may include the duration of the recording, the maximum, minimum and average heart rate, premature supraventricular complexes and any patient-triggered events, and/or any other metrics concerning the cardiac signal. Fifth section 185 may include information corresponding to any episodes detected. For example, fifth section 185 may include pause information (count and/or longest R-R interval), atrioventricular block information, atrial fibrillation/flutter information, ventricular tachycardia information, other supraventricular tachycardia information, and/or any other information concerning any episodes or abnormalities. Sixth section 186 may include results information such as, for example, a summary of the episodes and/or abnormalities, a diagnosis, and/or any other information analyzed, aggregated, computed, determined, identified, or otherwise detected from the cardiac signal. For example, sixth section 186 may identify a sinus rhythm with paroxysmal atrial fibrillation.

FIG. 15B-D illustrates the second, third and fourth pages of an exemplary report. As is shown in FIG. 15B-D, the report may further include ECG strips previously selected by the user, or selected under default settings. For example, a user may select Max HR strip 191, Min HR strip 192, Afib/Flutter strips 193, other SVT strips 194, PSVC strip 195, and PVC strip 196. Max HR strip 191 may be an ECG strip indicating the maximum heart rate during a given cardiac signal recording. Similarly, Min HR strip 191 may be an ECG strip indicating the minimum heart rate during a given cardiac signal recording. Afib/Flutter strips 193 may be ECG strips indicating each episode of atrial fibrillation/flutter. Other SVT strip 194 may be ECG strips indicating each episode of supraventricular tachycardia. PSVC strip 195 may be an ECG strip indicating an episode of premature supraventricular complex. PVC strip 197 may be ECG strips indicating episodes of premature ventricular complex. ECG strips may be displayed with the related relevant associated metrics and comments as added by the user. It is understood that the report shown in FIGS. 15A-B is merely exemplary and that the report generated at step 68 may have a different structure or configuration and/or may include different ECG and patient related information contemplated herein.

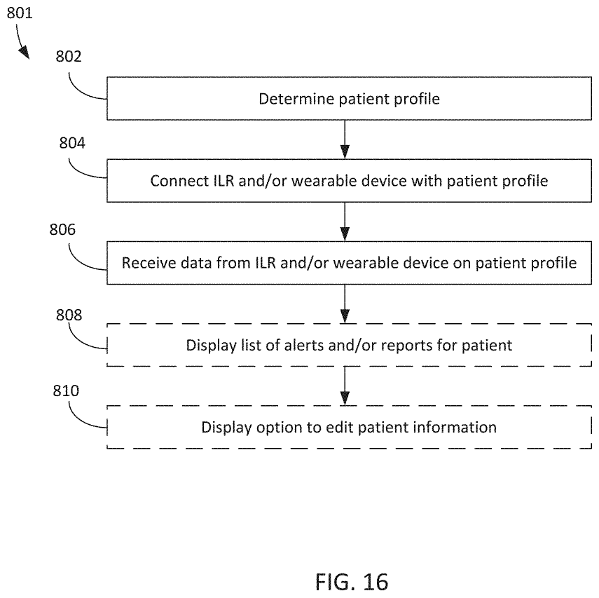

Referring now FIGS. 16-21C, the illustrated platform may be used by a user (e.g., physician, healthcare provider, technician), efficiently determine important data, to identify billable actions, tasks, and/or processes, and to label and/or classify certain actions, tasks, and/or processes for an electronic medical records (EMR) system. It is understood that the platform may be used for triaging data (e.g., classifying data as important or not), receiving and/or determining clinical decisions (e.g., writing a prescription, scheduling an appointment, etc.), determining certain billing information corresponding to the data (e.g., whether certain billing requirements for ILR monthly reports are satisfied). This may permit a physician, healthcare provider, and/or technician to bill for time related to an ILR and/or wearable device follow-up. Further the platform may generate a report that may be used to document a certain task and/or actions for the EMR. It is understood that the platform and the tasks and operations describe with respect to FIGS. 16-21C may be performed by ECG platform 37 illustrated in FIG. 3B.

Referring now to FIG. 16, an exemplary process for associating an implantable loop recorder (ILR) and/or wearable device (e.g., smart watch) with a patient profile on a platform is illustrated. For example, process 801 may be employed by a platform to determine ECG data from the ILR and/or wearable device, associate the ECG data from the ILR and/or wearable device with a patient profile, and determine alerts and/or reports corresponding to the data.

At block 802, a patient profile may be determined. For example, a user (e.g., physician, healthcare provide, and/or technician) may generate a profile for a particular patient. At block 804, a ILR and/or wearable device of a patient may be connected and/or associated with the patient profile such that data from the ILR and/or wearable device is periodically sent to and/or shared with the platform.

At block 806, the platform may receive data from the ILR and/or wearable device and may archive the data on a server and associate the data with the patient profile. For example, a server running the platform may receive data from the ILR and/or wearable device and may determine, based on a device identifier or a user identifier that the device is known and associated with a user profile and may archive that data in a manner that associates the data with the user profile.

At optional block 808 the platform may optionally display a list of the data, alerts and/reports based on the data. The platform may automatically generate alerts after processing the data using the techniques described herein (e.g., using delineation, classification, clustering, etc.). The platform may also automatically and/or at the direction of the user, generate reports corresponding to the data as described herein. At optional block 810, the platform may display an option to edit the patient information and/or any other information in the patient profile. For example, the user may alter the arrangement of the alerts and/or data displayed at optional block 808.

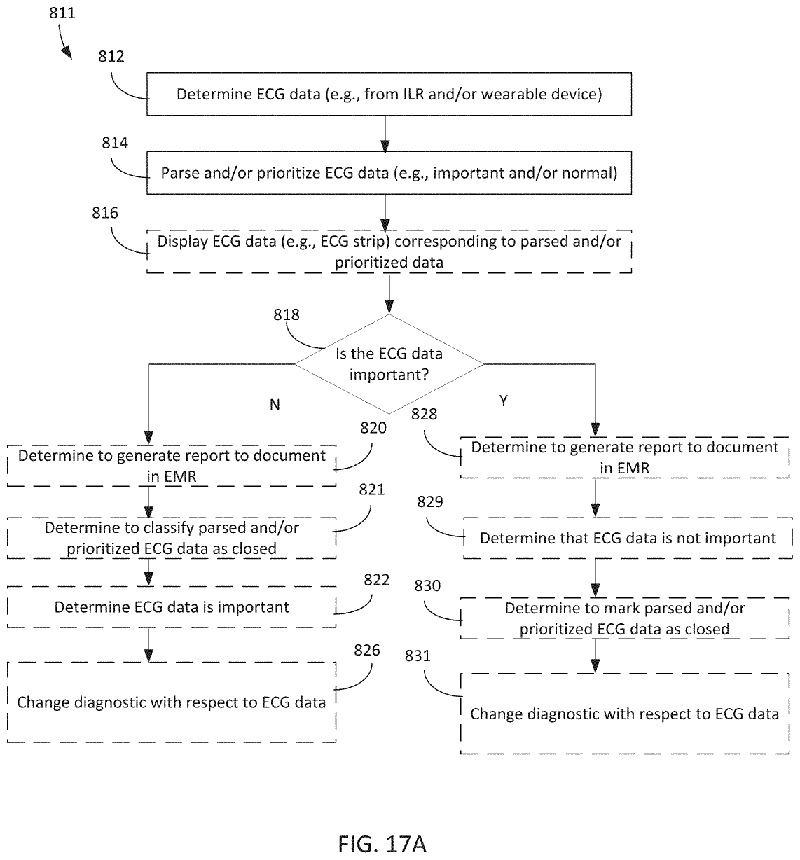

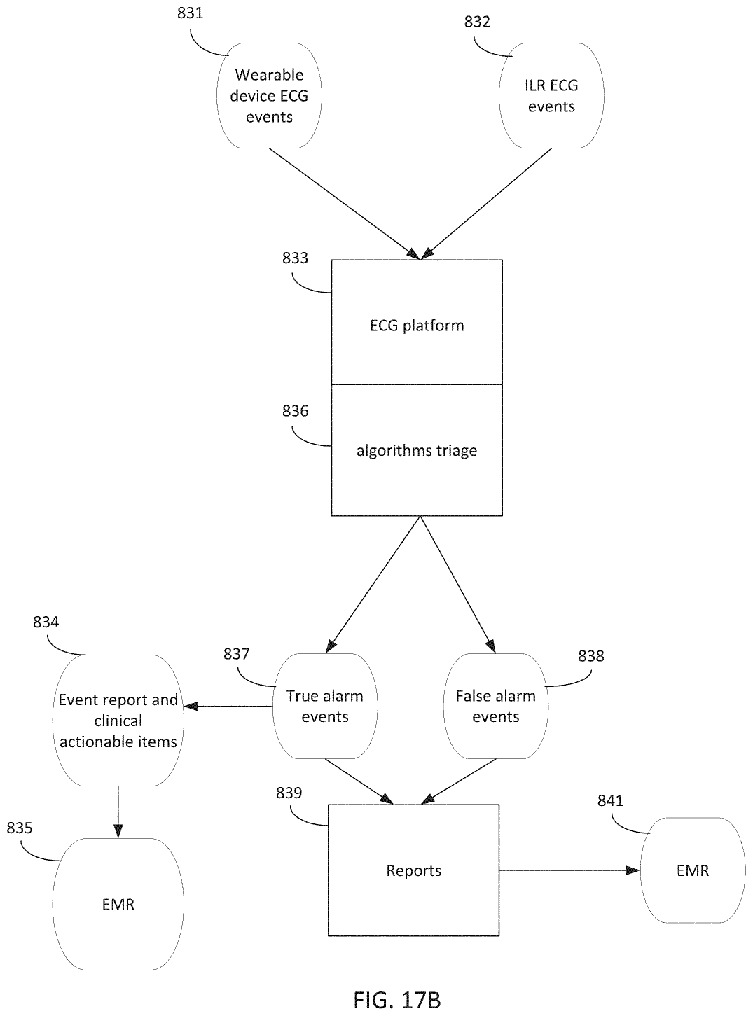

Referring now to FIG. 17A, an exemplary process for determining data from a loop recorder implantation (ILR) and/or wearable device (e.g., smart watch), determining if the data is important, and determining to take certain actions with respect to the data is illustrated. At block 812, the platform may determine ECG data (e.g., from a loop recorder implantation (ILR) and/or wearable device (e.g., smart watch). This may be the same step as step 806 of FIG. 16. At block 814, the data may be parsed and/or prioritized. For example strips of ECG may be determined and may be assigned a label as described herein (e.g., using delineation, classification, clustering, etc.). As shown in FIG. 17A, the ECG data may be determined to be either normal or important. The normal and/or important important label may be determined using the algorithms and techniques described herein and/or patient medical history and/or physician preference At optional block 816, the ECG data may be displayed based on the determination made at block 814. For example, ECG strips may be labeled as either important or normal. A user may elect to display the important and/or normal ECG strips.

At decision 818, if the ECG data is not important, at optional block 820, the platform (e.g., either automatically and/or at the direction of the user) may generate a report to document the important ECG data for EMR purposes. This may include generating a report as described herein. At optional block, the platform may determine to classify parsed and/or prioritized ECG data as closed. At optional block 822, the platform may further determine that the ECG data that was initially categorized as normal is important based on user feedback. For example, a user may view displayed ECG strips classified as normal and may instruct the platform that the ECG is important. At optional block 826, the user may change one or more diagnostics with respect to the ECG data.

If instead, at decision 818, the ECG data is important, the platform (e.g., either automatically and/or at the direction of the user) may generate a report to document the important ECG data for EMR purposes at optional block 828. This may include generating a report as described herein. At optional block 829, the platform may determine that the ECG data is not important (e.g., based on user feedback). At optional block 830, the platform may further determine to mark the parsed and/or prioritized ECG data and/or an event corresponding thereto as closed. For example, a user may view displayed ECG strips classified as important and may instruct the platform to mark the event and/or data as closed. At optional block 831, the user may change one or more diagnostics with respect to the ECG data.

Referring now to FIG. 17B, an exemplary data flow for determining ECG event data, determining alarms, and applying the data to EMR is illustrated. As shown in FIG. 17B, wearable device ECG events and/or ILR ECG events may be communicated to ECG platform 833, which may be the same as ECG processing system 10 and/or ECG platform 37. For example, ECG processing system 10 and/or ECG platform 37 may include algorithms triage module 836 which may determine whether ECG data and/or events are normal or important. As explained above with respect to FIG. 17A, an event may be normal even if there is noise. The ECG platform may process ECG events (e.g., ECG data) and classify it as important if it is abnormal (e.g., atrial fibrillation).

Based on the data received by ECG platform 836, true alarm events 837 and/or false alarm events 838 may be determined. For example ECG platform 836 may employ the techniques described herein (e.g., delineation, classification, clustering, etc.) to analyze wearable device ECG events 831 and/or ILR ECG events 832. True alarm events may correspond to the ECG platform correctly classifying the ECG event and/or data. False alarm events may correspond to the ECG platform incorrectly classifying the ECG event and/or data (e.g., based on user feedback). True alarm events and/or false alarm events may be used by reports module 839 to update EMR 841 and otherwise cause EMR 841 to incorporate this information.

The true alarm events may be used by the platform to generate item 834, which may include an event report and/or clinical action items. For example, ECG platform 833 may generate a report for important ECG events. The report may include ECG strips. Additionally, or alternatively, ECG platform may determine clinical actionable items and/or recommendations (e.g., in the form of a message and/or alarm). The information in item 834 may be used by and/or incorporated in EMR 835.

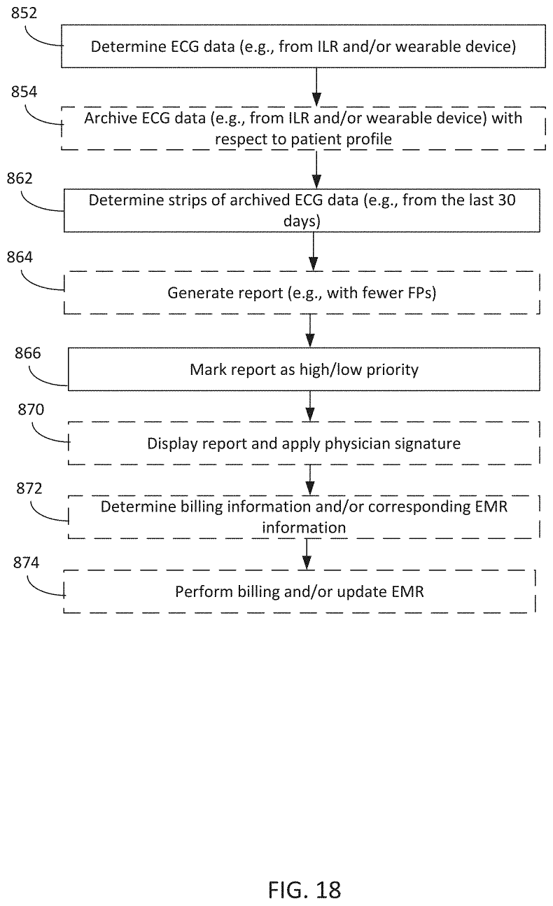

Referring now to FIG. 18, an exemplary process for determining data from a loop recorder implantation (ILR) and/or wearable device (e.g., smart watch), determining a priority and applying a physician signature to a report. At block 852, ECG data may be determined (e.g., from ILR and/or a wearable device). This step may be the same as step 812 of FIG. 17A. At optional block 854, the ECG data (e.g., from the ILR and/or wearable device) may be archived and/or otherwise saved (e.g., on a server). The data maybe be associated with a patient profile. At block 862 strips of archived ECG data may be determined. For example, a number of strips over a period of time may be determined (e.g., 30 strips over 30 days). At optional block 864, a report may be generated based on the ECG data (e.g., with fewer FPs).

At block 866, a report generated (e.g., at block 864) may be classified as a high or low priority. The priority designation may be assigned based on the presence of important information. The reports may include billing information and/or requirements, all ECG strips for a given period of time, and/or certain trends (e.g., HR trends). Alternatively, or additionally, a physician may review the report and determine the priority designation (e.g., high or low). At optional block 870, a report may be displayed and the platform may receive instructions to affix a signature to the report. At optional block 872, the platform may determine billing information and/or corresponding EMR information based on the report and/or data in the report. At optional block 874, billing may be performed based on information in the report and/or EMR may be updated such that relevant information from the report is applied to or otherwise incorporated into the EMR.

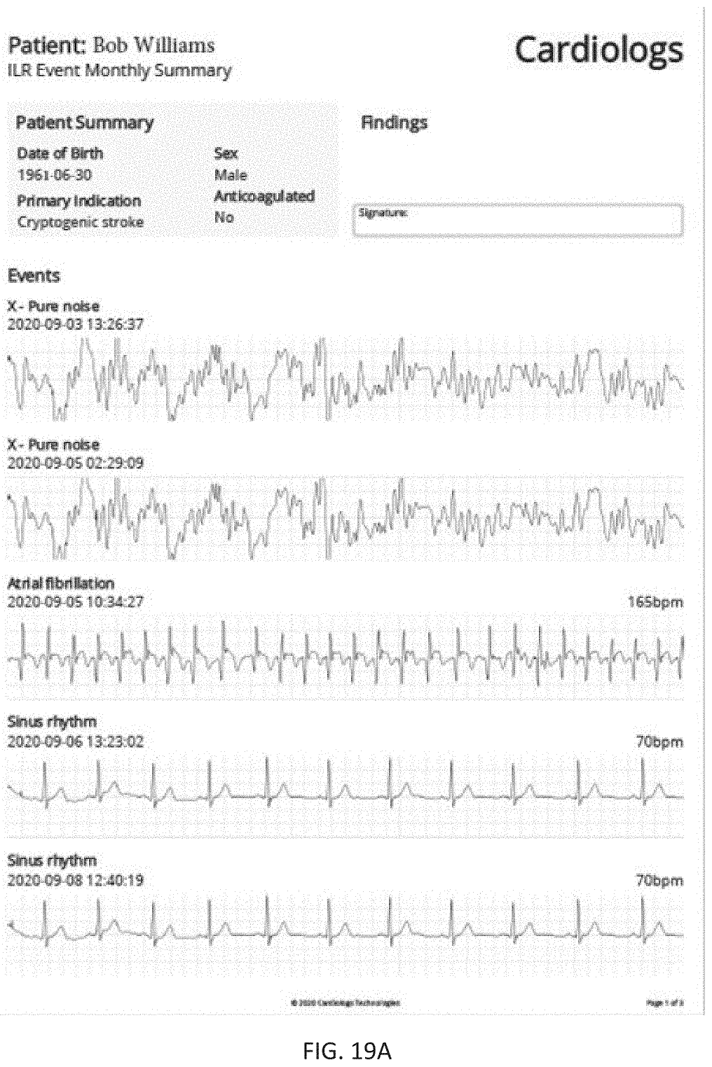

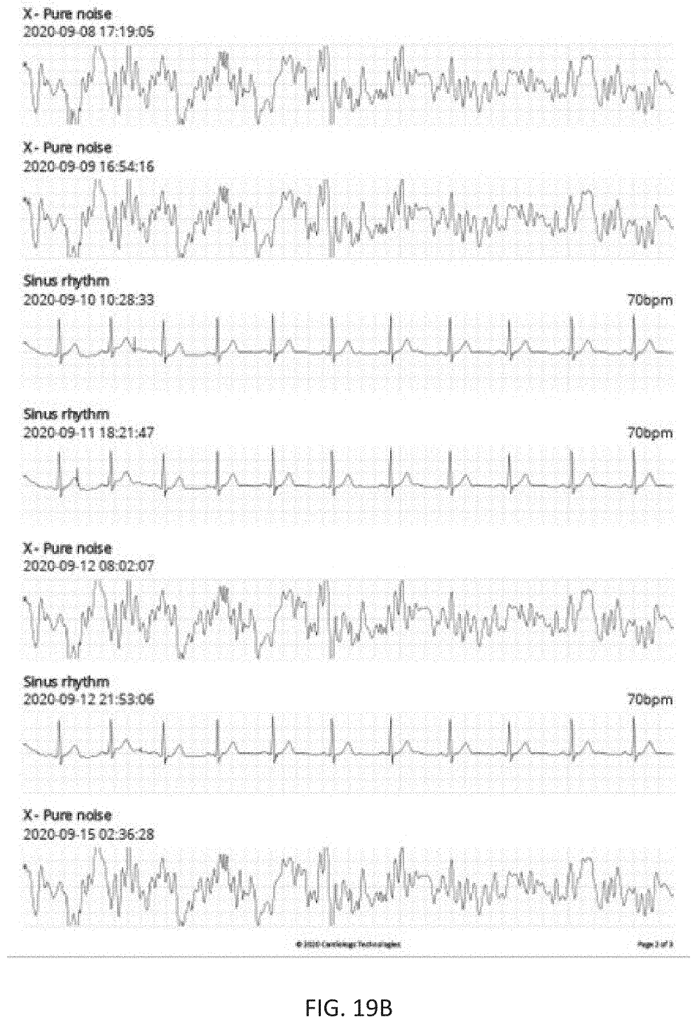

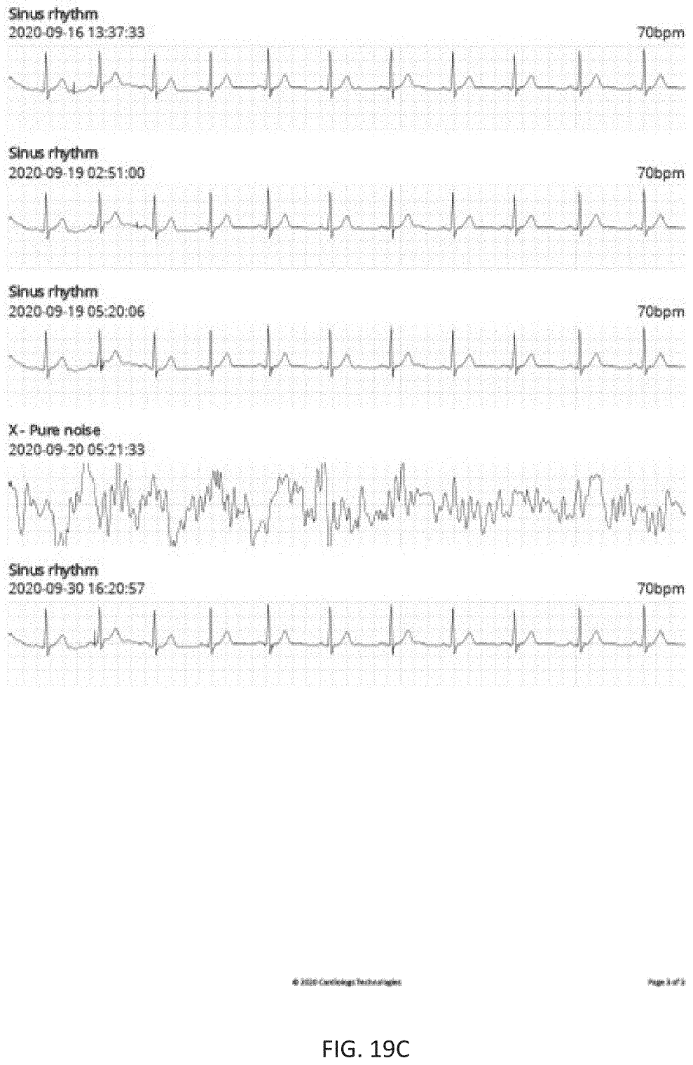

Referring now to FIGS. 19A-C an exemplary ILR/wearable device event report is illustrated. As shown in FIGS. 19A-C, the report may include patient information and ECG strips for various events (E.g., atrial fibrillation, sinus rhythm, etc.). While the report illustrates a month summary, it is understood that any other time frame may be included in a report. It is understood that the physician may add comments and/or sign the report.

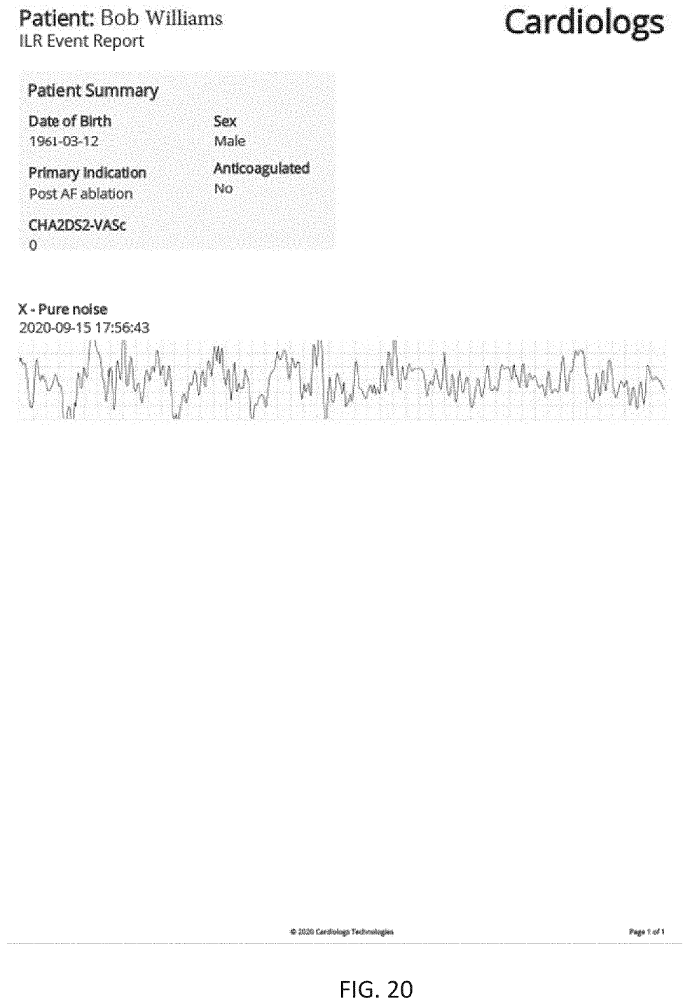

Referring now to FIG. 20, an exemplary ILR/wearable device event report is illustrated. The ILR event report may include information such as patient summary (e.g., including a primary indication) and/or an event ECG strip.

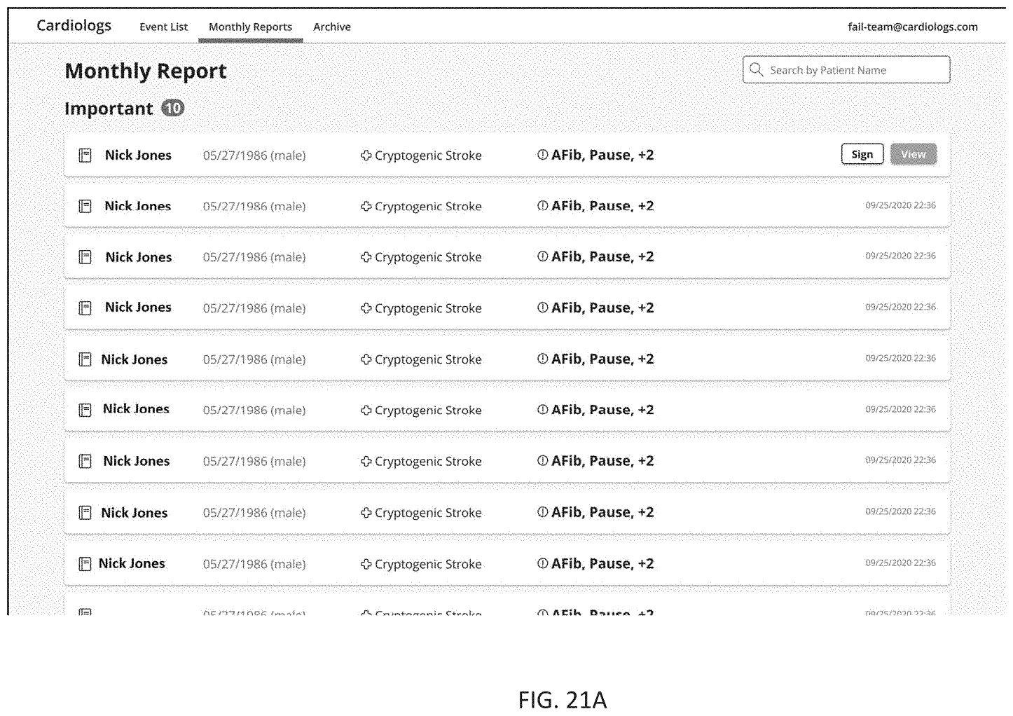

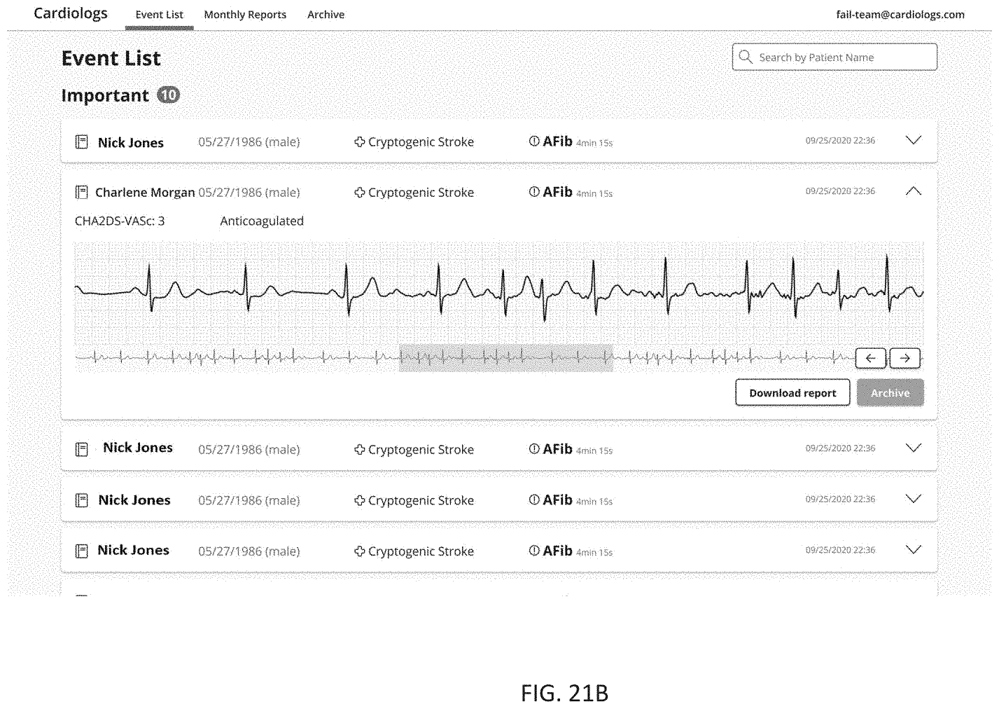

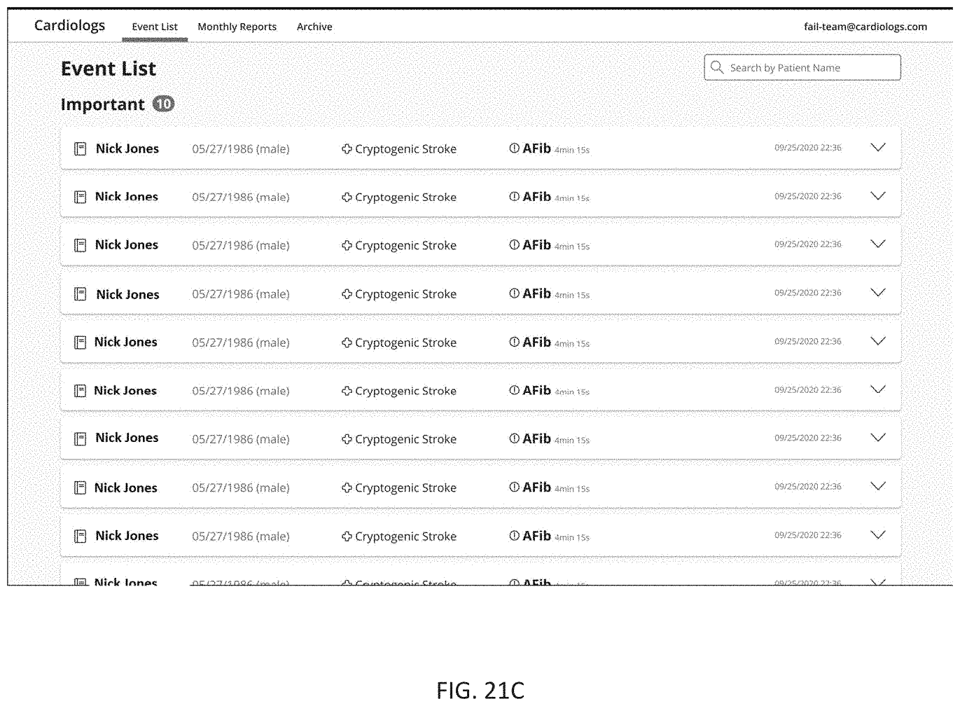

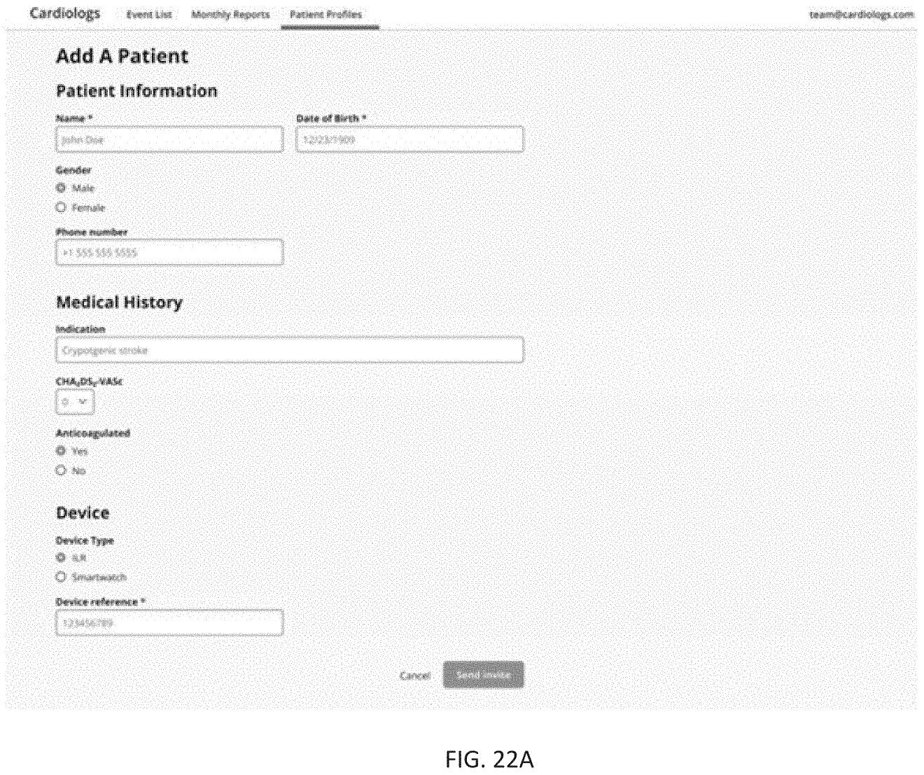

Referring now to FIGS. 21A-C, exemplary monthly report and event list user interfaces are illustrated. As shown in FIG. 21A, a monthly report may include a list of reports that have been identified as important and/or normal. Each event may include the patient's name, birthday, indication, event classification and/or description, and/or any other information (e.g., event data). Each report may be viewed and/or signed by a user, as described above with respect to FIG. 18. As shown in FIG. 21B, the platform may display an event list for events that are classified as important and/or normal. Each event may include the patient's name, birthday, indication, event classification and/or description, and/or any other information. The invention list may include one or more ECG strips for viewing the event. Each event in the event list may include the option to download a report, archive, and/or change priority level. As shown in FIG. 21C, the event list may optionally only include the patient's name, birthday, indication, event classification and/or description to streamline viewing.

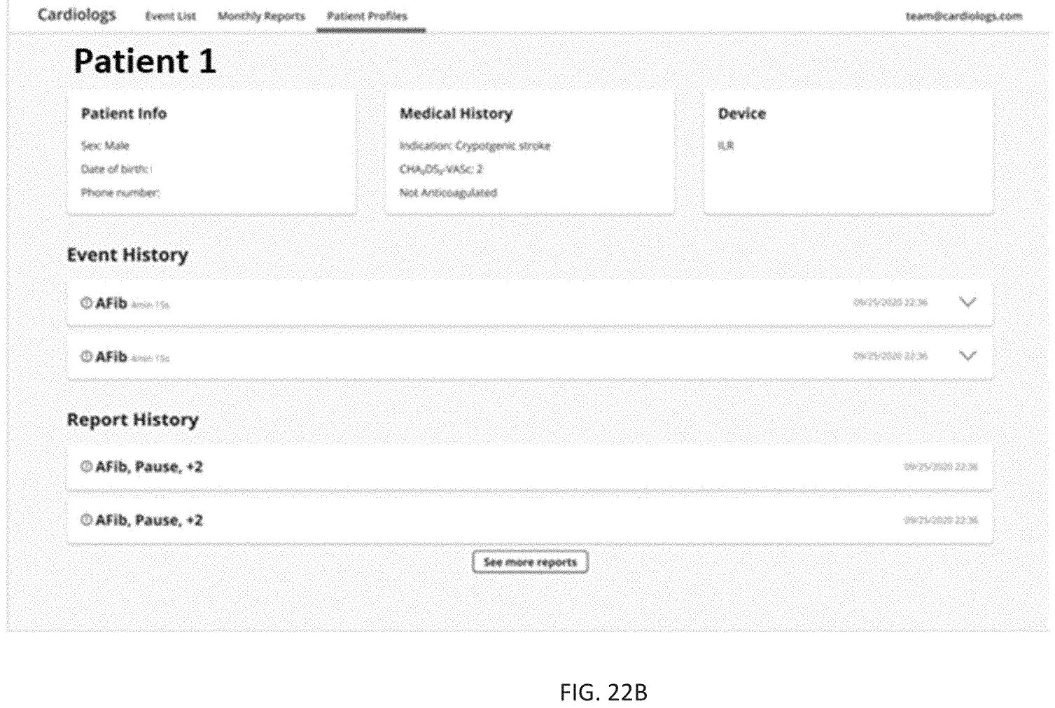

Referring now to FIGS. 22A-22B, exemplary user registration and profile interfaces are illustrated. As shown in FIG. 22A, an exemplary user registration interface may be used to add a patient and generate a user profile including the user name, date of birth, gender, contact information, medical history, device, and the like. As shown in FIG. 22B, an exemplary user profile may include patient information, medical history information, device information, event history, report history, and the like.

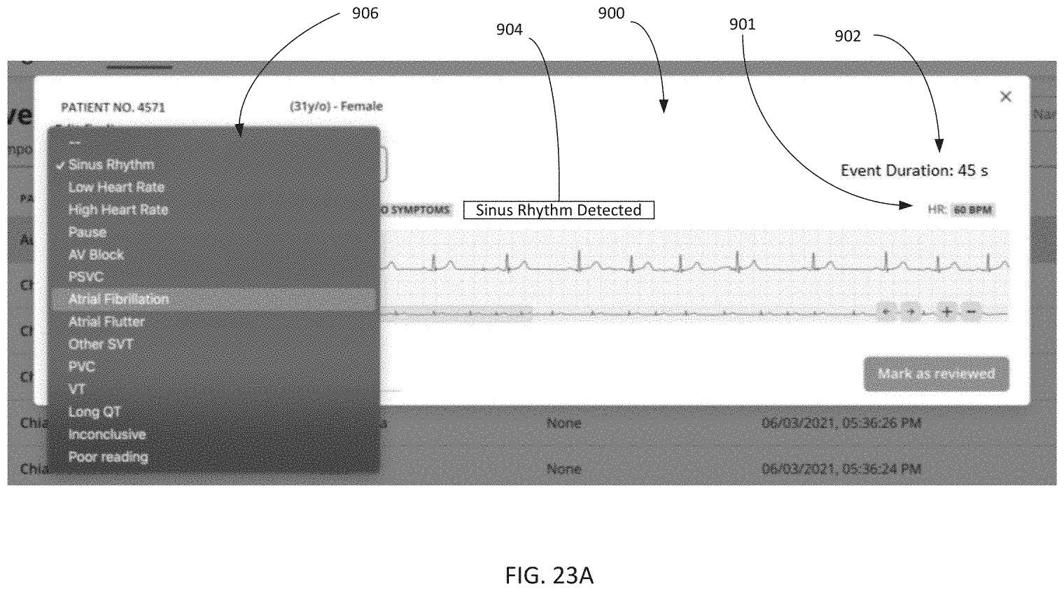

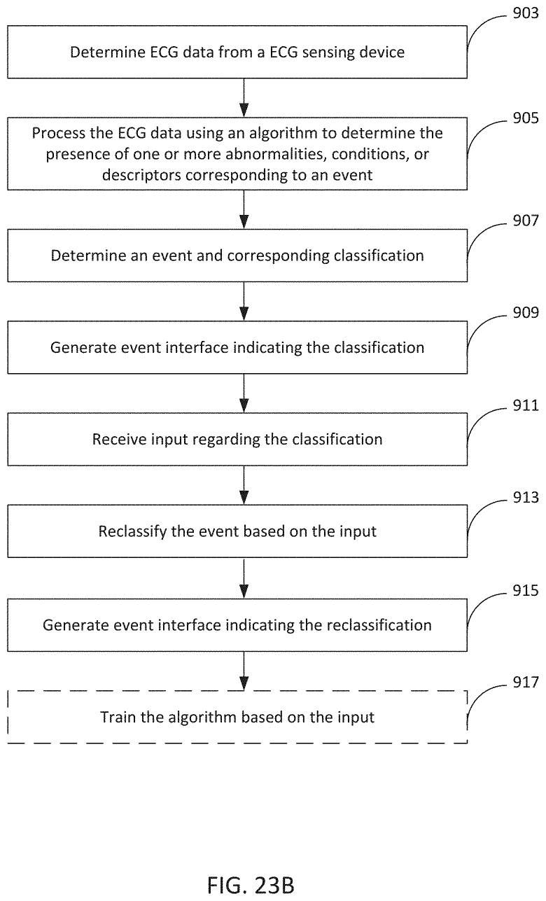

Referring now to FIGS. 23A-B, an exemplary event interface and process for reclassifying the event interface are illustrated. Referring now to FIG. 23A, event interface 900 is illustrated. Event interface 900 may display a portion of an ECG signal where an event was detected (e.g., using one or more approaches described herein). Event interface 900 may include heart rate indicator 901 which may display a detected or estimated heart rate corresponding to a point or interval of the ECG signal or alternatively an average, minimum, or maximum heart rate. Additionally, event interface 900 may include event duration 902, which may correspond to an event on-set and an event off-set. It is understood that any other relevant information (e.g., QTc) may displayed in event interface 900. Such information may be based on the delineation analysis described herein, for example.

FIG. 23A may further include classification box 904 and reclassification menu 906. Classification box 904 may display one or more classifications (e.g., conditions, abnormalities, descriptors, etc.) associated with the ECG signal. For example, classification box 904 may state “sinus rhythm detected.” Reclassification menu 906 may include a menu of selectable options for reclassifying the event detected in the ECG signal. For example, reclassification menu may include one or more of low heart rate, high heart rate, pause, AV block, PSVC, atrial fibrillation, atrial flutter, other SVT, PVC, VT, Long QT, or any other condition or abnormality. Reclassification menu 906 may further include additional classifications such as “inconclusive” and/or “poor reading.” By selecting an abnormality, condition or other information in reclassification menu 906, the event identified in event interface 900 may be reclassified. The reclassified event may be used to train the algorithms, neural network architectures, and models used to initially classify the event.

Referring now to FIG. 23B, an exemplary process for generating (e.g., by the ECG platform) an event interface including a classification of the event and reclassifying the event based on the event interface is illustrated. Some or all of the blocks of the process in FIG. 23B may be performed in a distributed manner across any number of devices (e.g., computing devices and/or servers). Some or all of the operations of the process in FIG. 23B may be optional and may be performed in a different order.

To initiate the process set forth in FIG. 23B, at step 903, ECG data from an ECG sensing device (e.g., ILR) is determined and/or obtained. At step 905, the ECG data may be processed using an algorithm to determine the presence of one more abnormalities, conditions, or descriptors corresponding to an event (e.g., cardiac event, ECG event, and/or any other physiological event). At step 907, one or more classifications corresponding to theevent may be determined using the algorithm. For example, the classification “sinus rhythm” may be determined based on the presence of one or more abnormalities, conditions, or descriptors.

At step 909, an event interface may be generated indicating (e.g., displaying) the classification and/or cardiac event determined at step 907. For example, the event interface may display “sinus rhythm” and may include a representation of the ECG signal corresponding to the event. At step 911, input regarding the classification may be received. For example, a system device (e.g., healthcare provider device) may present the event interface and the healthcare provider may send the ECG platform a message regarding the classification (e.g., regarding the accuracy of the classification).

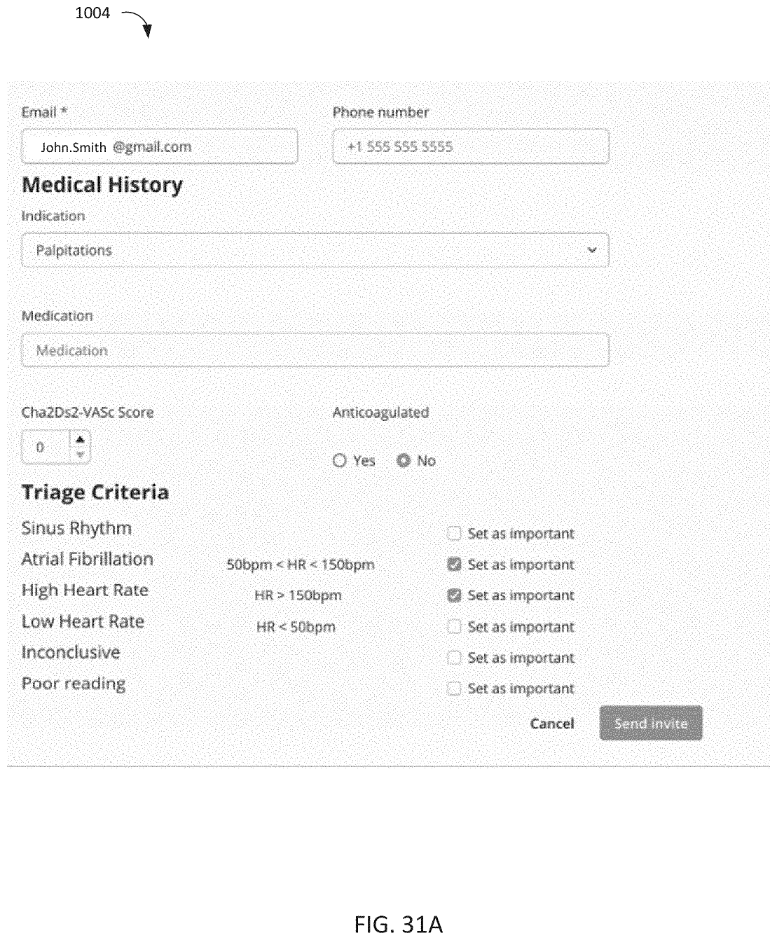

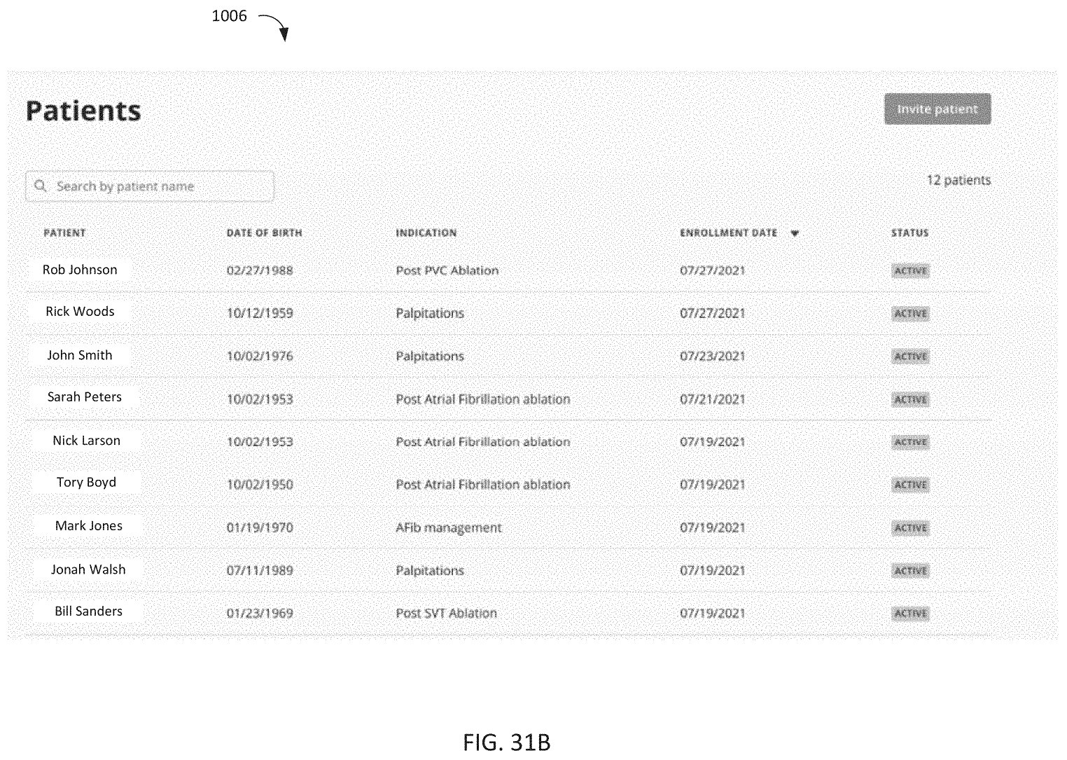

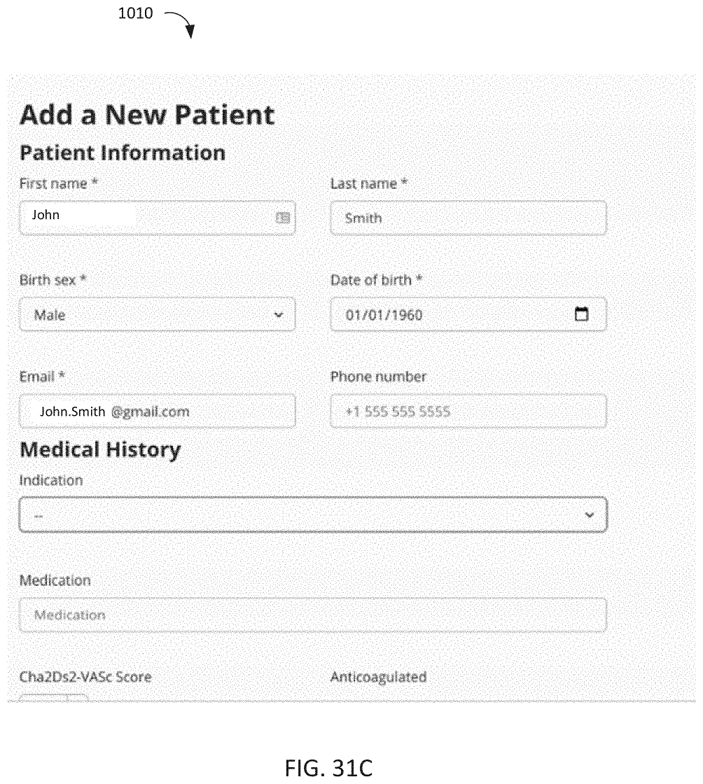

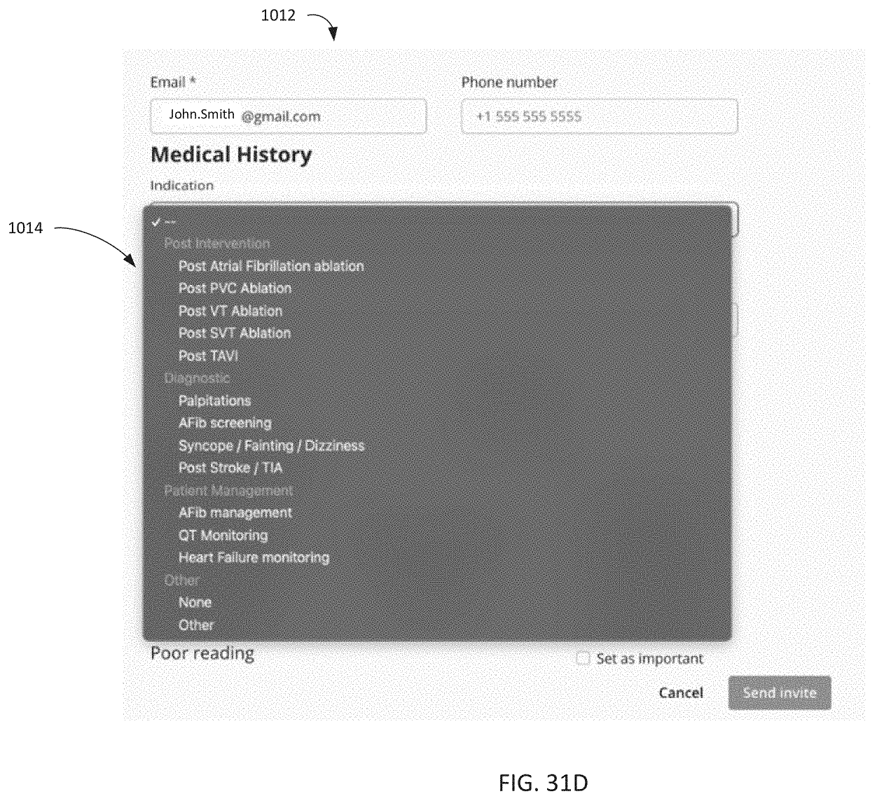

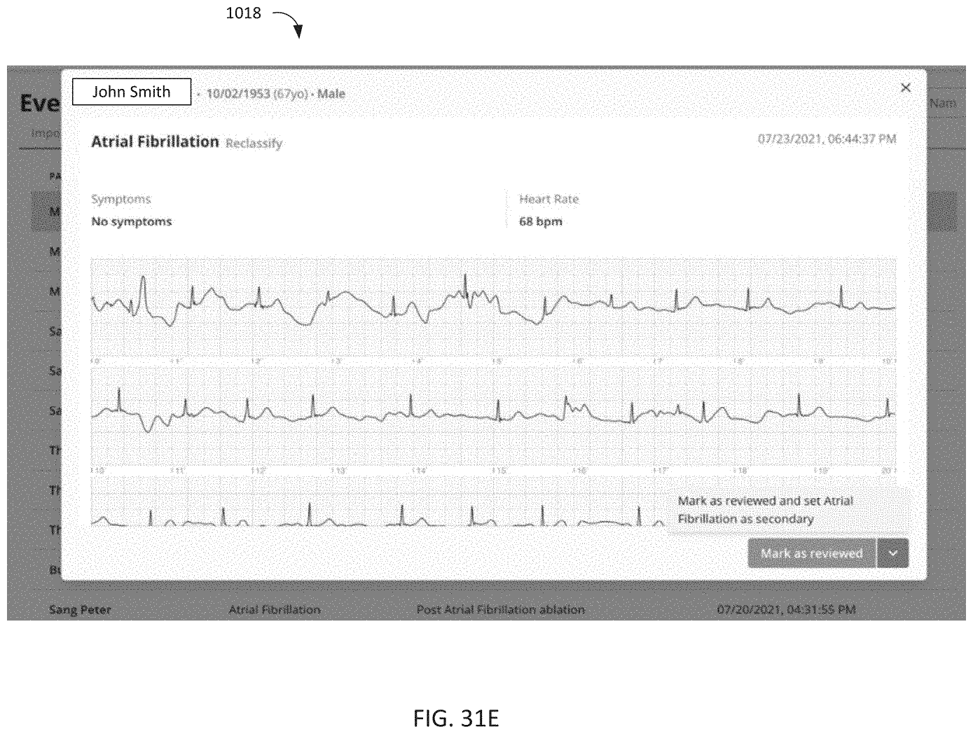

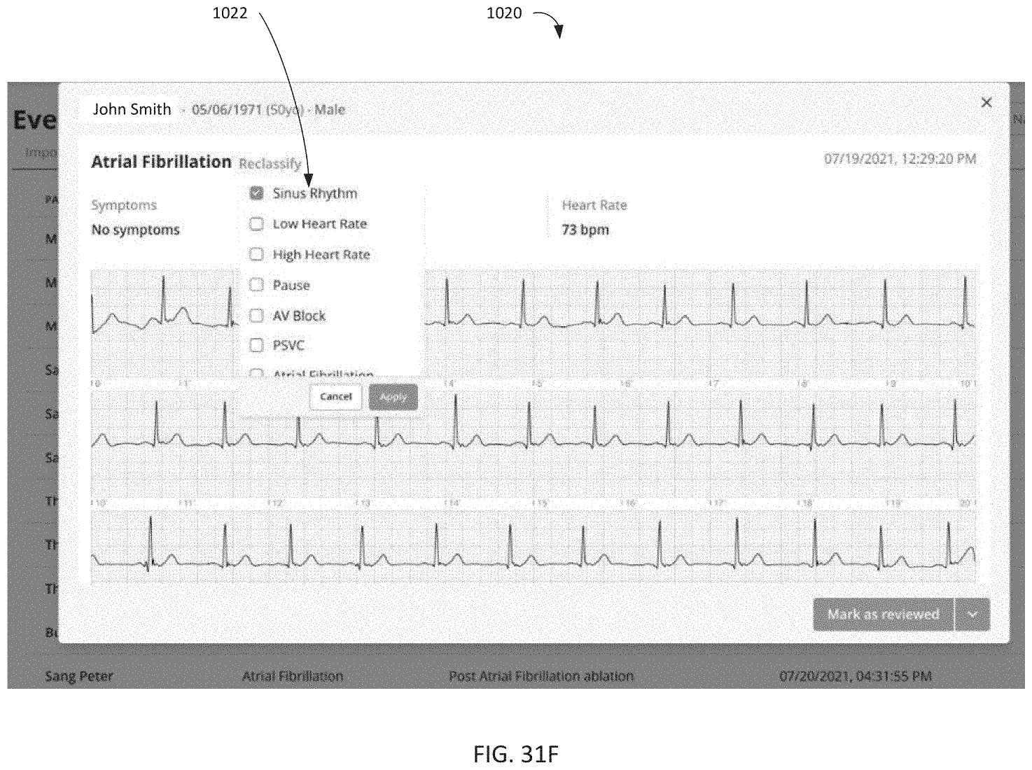

At step 913, the cardiac event may be reclassified based on the input received. For example, the input may indicate that the classification determined at step 907 was not accurate and may even identify a new classification. The new classification may be used to reclassify the event. At optional step 915, an event interface may be generated indicating the reclassification determined at step 913. At optional step 917, the algorithm used to process the ECG data at step 905 may be trained and/or otherwise modified based on the reclassification. Event interfaces and reclassification are described in greater detail below with respect to FIGS. 31E-31F.

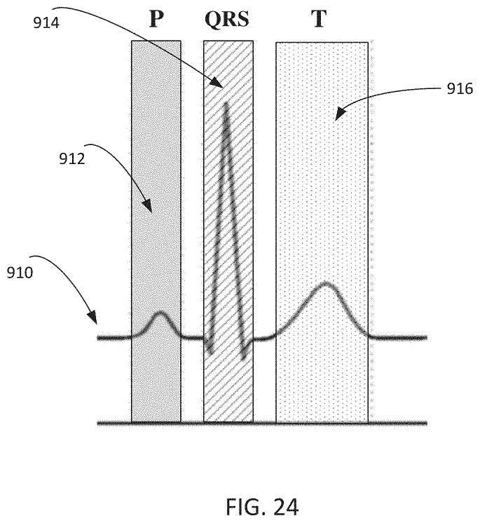

Referring now to FIG. 24, an exemplary ECG signal with color bands is illustrated. Specifically, ECG display 910 may be a portion of the ECG signal displayed in the event interface illustrated in FIG. 23A and/or any other presentation of an ECG signal and may include color indictors 912, 914, and 916. Color indicator 912 may be any color and/or pattern different from color indicators 914 and 916 and may indicate this portion of the ECG signal corresponds to a p-wave, for example. Color indicator 914 may be any color and/or pattern different from color indicators 912 and 916 and may indicate that this portion of the ECG signal corresponds to a QRS complex, for example. Color indicator 916 may be any color and/or pattern different from color indicators 912 and 914 and may indicate that this portion of the ECG signal corresponds to a t-wave, for example. It is understood that any color or pattern may be used to differentiate various portions of the ECG signal. It is further understood that color indicators may be used to indicate any portion and/or feature of an ECG signal (e.g., hidden p-wave, QT interval, ST segment, RR interval, TP segment, PR segment, and the like). The color indicators may be based on the delineation analysis and/or any other analysis described herein.

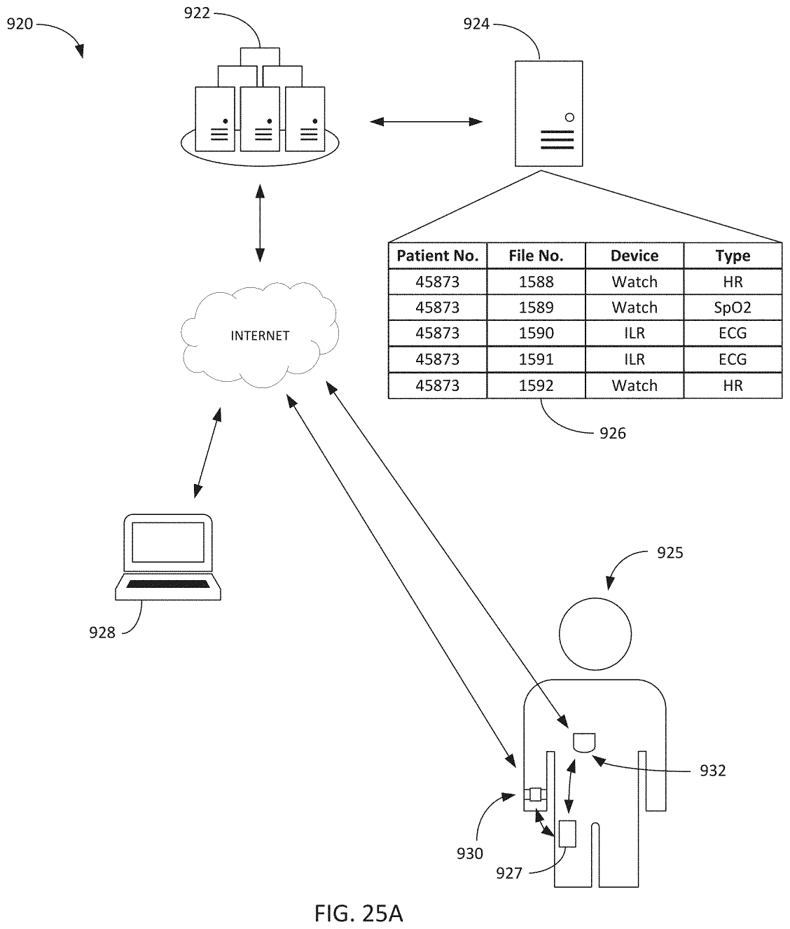

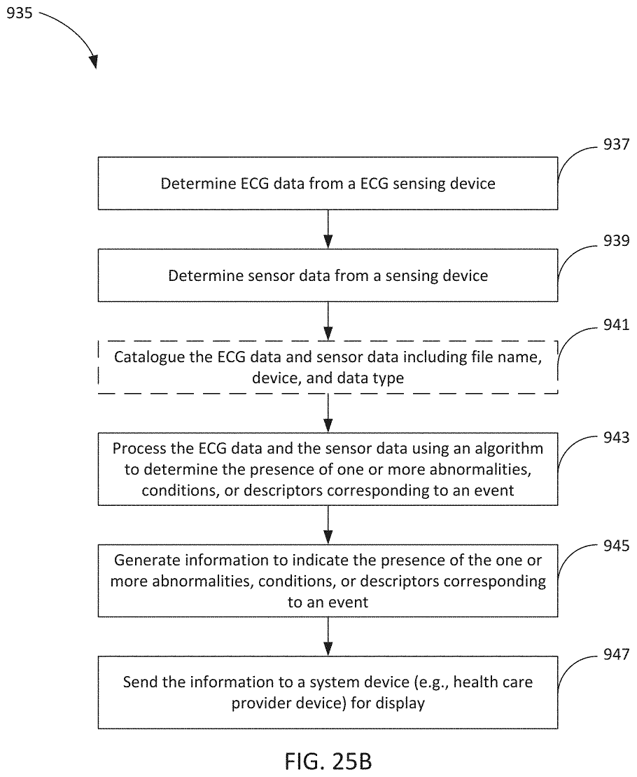

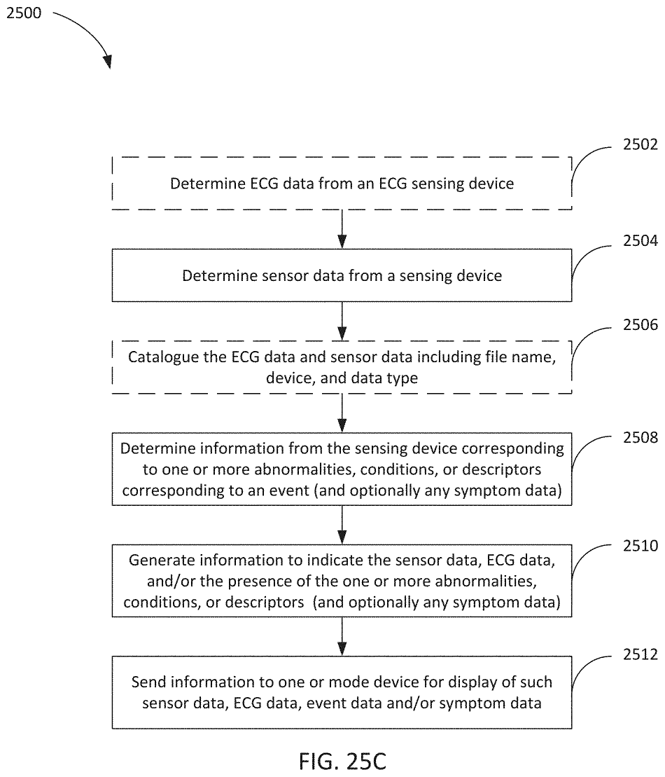

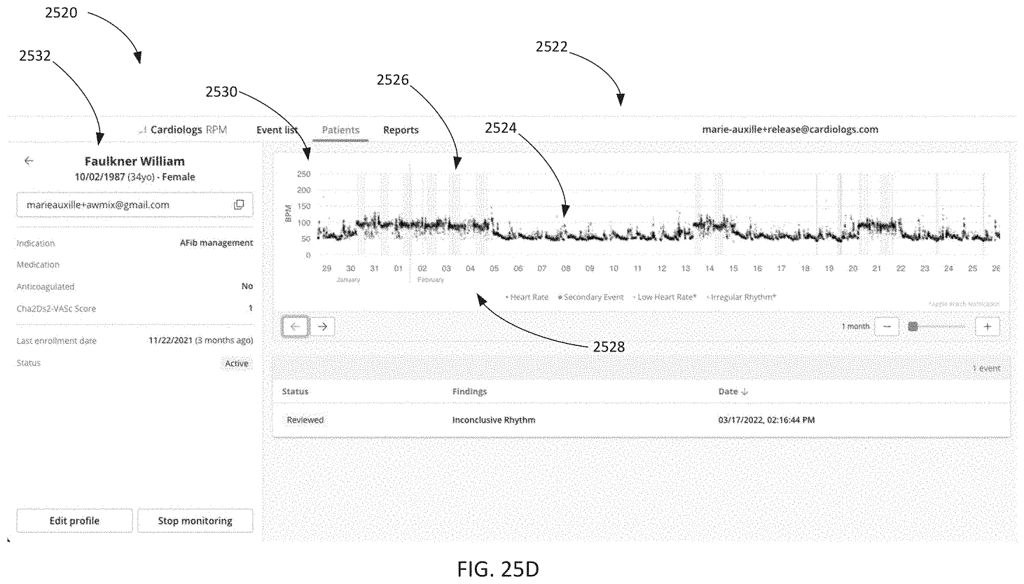

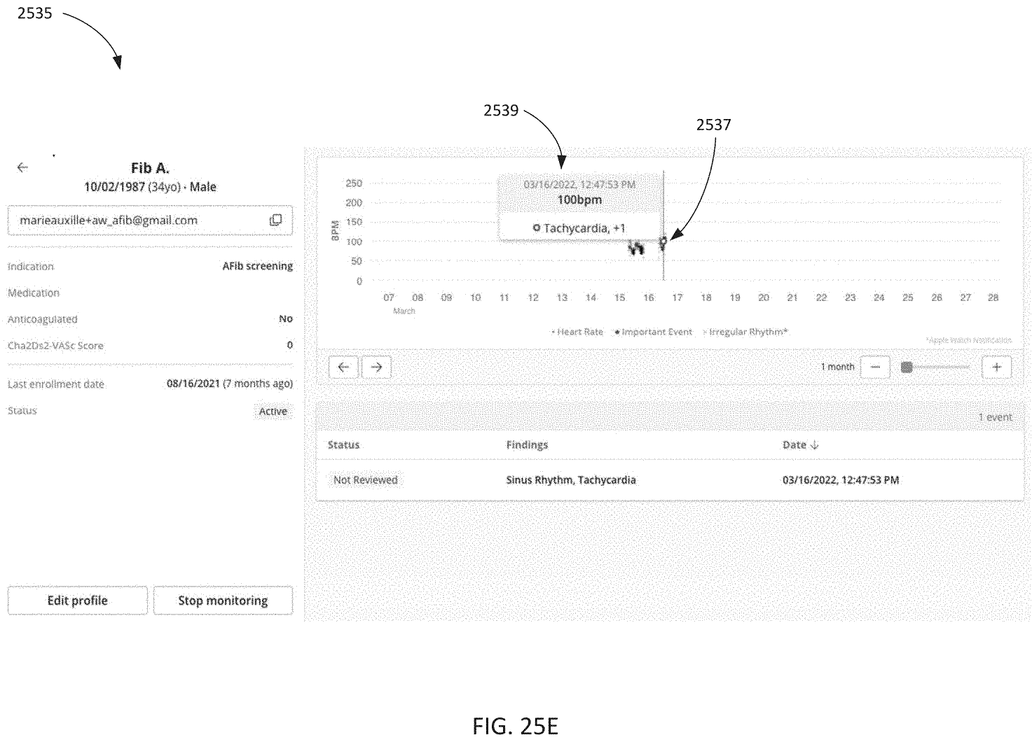

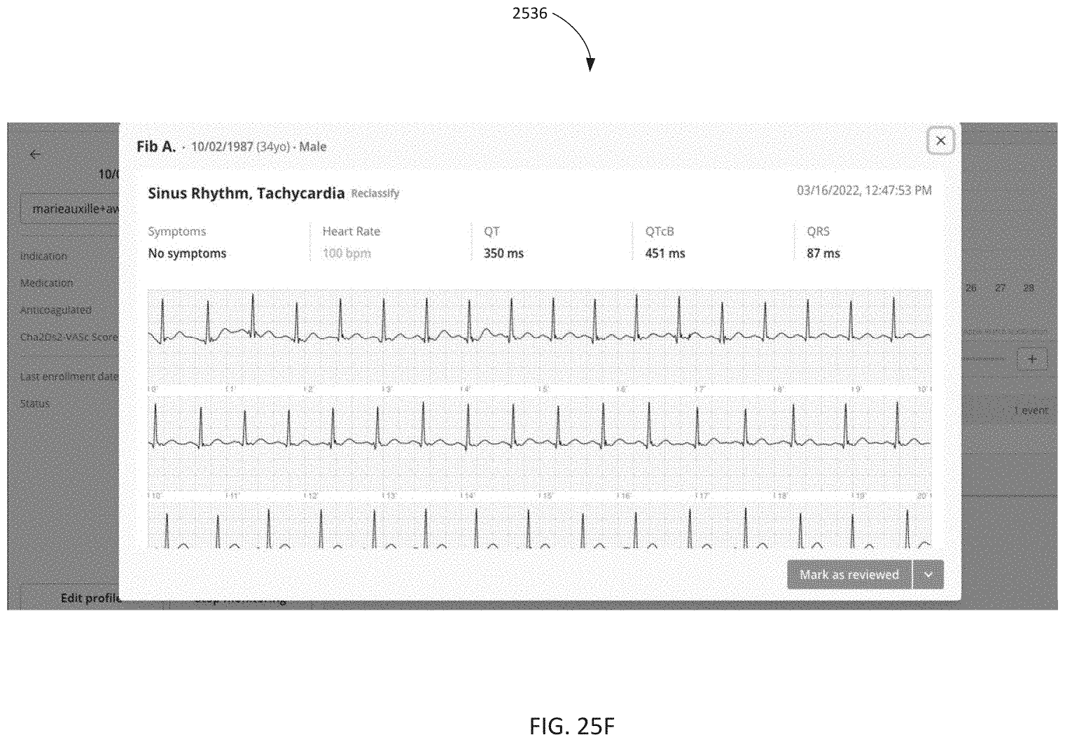

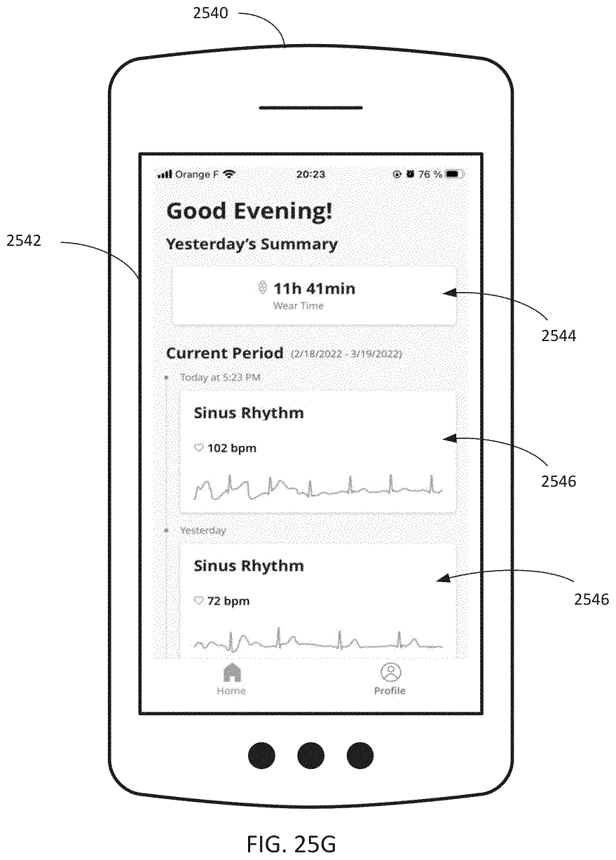

Referring now to FIGS. 25A-C, an exemplary system and process for multi-device ECG processing is illustrated. Referring now to FIG. 25A, ECG processing system 920 may include server 922, drive 924, mobile device 927, system device 928, sensing device 930, and sensing device 932. Server 922 may be the same or similar to server 15 described above with respect to FIG. 2 and may run an ECG platform (e.g., ECG platform 37 described above with respect to FIG. 3A). Drive 924 may be the same or similar to drive 16 described above with respect to FIG. 2. System device 928 may be the same or similar to system device 14 described above with respect to FIG. 2. Sensing device 930 and/or sensing device 932 may be similar to sensing device 13 described above with respect to FIG. 2. Drive 924 may be incorporated into server 922 or may be separate and distinct from server 922 and/or may communicate with server 922 over any well-known wireless or wired connection. System device 928 may be in communication with server 922, sensing device 930 and/or sensing device 932 via any well-known wireless or wired connection. Further, sensing device 930 and/or sensing device 932 may be in communication with server 922 and/or system device 928 via any well-known wireless or wired connection. Sensing device 930 and sensing device 932 may optoinally be in communication with mobile device 927 via any well-known wireless or wired connection. Mobile device 927 may also be in communication with system device 928 and/or server 922 via any well-known wireless or wired connection. Any other elements of ECG processing system 920 may also be in communication via any well-known wireless or wired connection as well.

Sensing device 930 and sensing device 932 may be any type of device for sensing electrical activity of the heart, generating ECG data (e.g., ECG signals), and/or generating any other biometric or physiological data (e.g., heart rate, temperature, motion, oxygen levels (SpO2), respiratory rate, humidity, blood pressure, etc.). Sensing device 930 and sensing device 932 may be the same or different devices. For example, sensing device 930 may be a smart watch worn by user 925 and sensing device 932 may be an implantable ECG recording device (e.g., ILR). While only two sensing devices are illustrated in FIG. 25A, it is understood that processing system 920 may include more than two devices. Sensing devices may include other wearable devices and/or implantable devices.

Sensing device 930 and sensing device 932 may generate sensed data (e.g., ECG data and/or other biometric or physiological data) and may send such data to server 922 either directly or indirectly. For example, sensing device 930 and sensing device 932 may send the data to mobile device 927 and mobile device 927 may send the data to server 922. Alternatively, or additional, sensing device 930 and sensing device 932 may send the data directly to server 922 or may send the data to server 922 via a computing device such as system device 928. Upon receiving the sensed data, server and/or drive 924 may analyze the data using one or more approaches or techniques described herein (e.g., process the sensed data to determine an anomaly, abnormality or condition). System device 928 may be used to analyze and otherwise oversee processing and analyzing the sensed data on server 922.

Mobile device 927 may be any type of device, such as a smart phone (as one non-limiting example). Sensing device 930 and sensing device 932 may send data (for example, ECG data, heartbeat data, and/or any other data determined and/or obtained by sensing device 930 and sensing device 932) to mobile device 927 and/or server 922. Mobile device 927 may run a mobile application in communication with an application run on server 922, may also receive results of any analyses performed by server 922 or a user associated with server 922, and/or may present these results through a user interface associated with an application installed on the mobile device. For example, a user's smart phone may receive data from a smart watch worn by the user. The user's smart phone may communicate the data to a server and may access the data from the server and/or any analyses performed on the server. Mobile device 927 may also present any other types of information, such as any data received from sensing device 930, sensing device 932, and/or any other sensing device. It should be noted that this is merely one example use case and is not intended to be limiting in any way.

As shown in FIG. 25A, drive 924, which may be incorporated into server 922, may maintain databases such as database 926 to keep track of the different types of sensed data received from the various sensing devices (e.g., sensing device 930 and sensing device 932). For example, database 926 may assign a name (e.g., file name) to each of the received data and may associate the file name with the user or user account (e.g., patient no.) and may even identify the device that provided and/or generated the data as well as the type of data (e.g., heart rate (HR), SpO2, ECG, etc.). It is understood that ECG processing system 920 may include one or more sensing device 930, one or more sensing device 932 and/or combination of one or more sensing devices 930 and 932. It is understood that the sensed data generated by the sensed devices and received by the server may be data other than ECG data, such as heart rate, respiratory rate, and other non-ECG data.

Referring now to FIG. 25B, a process for analyzing ECG and other data generated by a multi-device system for determining conditions, abnormalities, and/or descriptors is illustrated. To initiate multi-device process 935 (e.g., on an ECG platform), at step 937, ECG data from a sensing device is obtained and/or determined over a given time period (e.g., at a given sampling rate). At step 939, sensor data from a different sensing device (e.g., a smart watch or any other sensing device) is obtained and/or determined over a given time period. The sensor data may be any type of well-known physiological or biometric data (e.g., heart rate, SpO2, respiratory rate, etc.). In one example, the sensor data is generated by a photoplethysmogram (PPG) sensor. The time period for the sensor data and the ECG data may be the same or may overlap, even if the sampling rates are different.

At optional step 941, the ECG data and the sensor data may be catalogued or otherwise saved in an organized fashion (e.g., in a database) such that the ECG data and sensor data may be associated with the device from which it originated, the type of data, a file number, and/or any other information relevant to the ECG and/or sensor data. At step 943, the ECG data and sensor data may be processed using an algorithm to determine the presence of one or more abnormalities, conditions and/or descriptors corresponding to an event (e.g., cardiac event, ECG event, and/or any other type of physiological event). For example, techniques and/or algorithms similar to those described above (e.g., the techniques and/or algorithms described above with respect to FIG. 4) may be employed to analyze and/or process the sensor data and/or ECG data. It is understood that the various algorithms, neural networks, and models described above (e.g., the delineator and classifier) may be trained and/or otherwise designed to process both ECG data and other sensor data.

At step 945, information indicative of the presence of the one or more abnormalities, conditions, or descriptors corresponding to the event may be generated. For example, such information may be used to generate a display on a system device and/or generate a report regarding the one or more abnormalities, conditions, or descriptors. At step 947, the information generated at step 945 may be communicated to a system device for display. For example, the information may be sent or otherwise accessed by a health care provider device for display on the healthcare provider device.

Referring now to FIG. 25C, process 2500 for receiving and displaying ECG and/or other data (e.g., heart rate data) generated by one or more device for is illustrated. To initiate process 2500 (e.g., on an ECG platform), at optional step 2502, ECG data from a sensing device may be obtained and/or determined. At step 2504, sensor data from a different sensing device (e.g., a smart watch or any other sensing device) and/or the same sensing device in step 2502 may be obtained and/or determined. The sensor data may be any type of well-known physiological or biometric data (e.g., heart rate, SpO2, respiratory rate, etc.). In one example, the sensor data is generated by a photoplethysmogram (PPG) sensor. It is understood that the time period over which the sensor data is generated and the time period over which the ECG data is generated may be the same or may overlap, even if the sampling rates are different.

At optional step 2506, the ECG data and/or the sensor data may be catalogued or otherwise saved in an organized fashion (e.g., in a database). For example, that the ECG data and/or sensor data may be associated with the device from which it originated, the type of data, a file number, and/or any other information relevant to the ECG and/or sensor data. At step 2508, information may be obtained and/or determined from the sensing device corresponding to one or more abnormalities, conditions, or descriptors corresponding to an event. For example, the sensing device may communicate information indicating the presence of atrial fibrillation. Optionally, the sensing device may communicate one or more symptoms or other health related information corresponding to the patient. For example, the sensing device may communicate information indicating that the patient experienced heart palpitations.

At step 2510, information indicative of the sensor data, ECG data, and/or presence of the one or more abnormalities, conditions, or descriptors corresponding to a cardiac event may be generated. Information indicative of symptom data may optionally be generated as well. For example, such information may be used to generate and/or render a display for presentation on a device based on the information and/or cause a display to generate and/or render such a display. At step 2512, the information generated at step 2510 may be communicated to a mobile device and/or any other computing device for display and/or presentation. For example, the information may be sent to a smart phone of a user so that the user may view the information through a user interface of an application installed on the mobile device.

Referring now to FIG. 25D, user interface 2520 for displaying ECG data and other sensor and healthcare data is illustrated. User interface 2520 may display one or more plots 2522 including one or more data points 2524. Data points 2524 may be, be based on, or may represent heart rate data, ECG data, and/or any other types of cardiac and/or healthcare data. For example, plot 2522 may display data that is received from a sensing device associated with a user, such as a smart watch and/or any other type of sensing device that may determine heart rate data. An x-axis 2528 of plot 2522 may represent time and a y-axis 2530 of plot 2522 may represent a value associated with sensor data and/or healthcare data (e.g., heart rate heart rate value, an ECG reading, etc). Plot 2522 may also be associated with various notifications and/or alerts. Non-limiting examples of alerts may include high and low heart rate, irregular rhythm, etc. The data in plot 2522 may be determined by one or more sensing device. The alerts and/or notifications may be visually indicated to the user through one or more indicator lines 2526 and/or any other well-known visual techniques (e.g., markers, highlighting, symbols, etc.). Indicator line 2526 may provide an indication to the user that a specific data point or group of data points are indicative of an abnormality, condition, or descriptor associated with an event. A color of indicator line 2526 may correspond to a specific abnormality, condition, or descriptor. The notifications and/or alerts may also be presented in any other form other than indicator lines as well. Data points 2524 in plot 2522 may also be filtered so that the user may choose to view only a specific type of data (e.g., heart rate data, PPG data, ECG data, etc.).

Additionally, a user may also be able to interact with plot 2522. For example, a user may be able to zoom in to view a particular portion of plot 2522 in more detail, may be able to select one or more of data points 2524 to view additional information about individual data points or groups of data points, and/or may be able to perform any other types of interactions with plot 2522. For example, selecting a data point corresponding to a certain heart rate may generate a graphical representation of an ECG signal corresponding to that data point. The user may not be required to select a data point for additional information to be displayed. For example, a user may simply hover a mouse cursor over a data point for additional information to be displayed in a pop-up window and/or in any other format. One non-limiting example of information may include a medical determination based on ECG data, an average heart rate, etc. The types of information that is presented may vary depending on the types of data that are included in plot 2522 (for example, heart rate data, ECG data, etc.). The user may also customize types of information that is displayed as well. The user may also be able to interact with indicator line 2526 to view more specific information about an abnormality, condition, or descriptor associated with indicator line 2526.

User interface 2520 may also present profile information 2532. For example, profile information 2532 may include personal information associated with the user, any medication that is prescribed to the user, any information about any identified or previously-determined abnormalities or other conditions associated with the user, and/or any other types of relevant data as illustrated in the figure or otherwise. It should be noted that the information illustrated in the figure is merely exemplary and is not intended to be limiting. That is, any other relevant information may also be presented in user interface 2520.

Referring now to FIG. 25E, user interface 2535 for displaying additional information relating to individual ECG data points and/or other data points is illustrated. It is understood that user interface 2520 may be the same or similar to user interface 2535. For example, a user may be able to select a data point 2537 (or group of data points) to view additional information about data point 2537 (or groups of data points). Upon selection, a pop-up box 2539 may appear that includes this additional information. As one non-limiting example, the pop-up box 2539 may include a time at which data point 2537 was captured and a value associated with data point 2537 (for example, if data point 2537 relates to heart rate readings for a user, then the pop-up box 2539 may present the numerical value of heart rate reading associated with data point 2537). It should be noted that the information may also be presented in any format other than a pop-up box as well. Additionally, a user may not be required to “select” data point 2537 for the additional information to be displayed. For example, a user may simply hover a mouse cursor over data point 2537 for the information to display.

Referring now to FIG. 25F, user interface 2536 for displaying a graphical representation of ECG data relating to one or more ECG data points and/or other data points is illustrated. It is understood that user interface 2536 may be the same or similar to user interface 2520 of FIG. 25D. For example, a user may select a data point (e.g., ECG data point) in the user interface illustrated in FIG. 25D to view additional ECG information about such data and user interface 2536 may be generated to display an ECG representation related to the data selected. The ECG representation may be generated from data used to create the user interface illustrated in FIG. 25D and/or may be received from one or more sensing devices. User interface 2536 may additionally include information about the patient (e.g., name, age, sex), identified anomalies, conditions, events, descriptors, any symptoms, heart rate, and other ECG characteristics such as QT value, QTcB value and/or QRS value for the selected data and/or ECG representation.

Referring now to FIG. 25G, a mobile device 2540 presenting a mobile interface 2542 is illustrated. Mobile device 2540 may be any type of computing device having a processor and a display and in communicate with a server running an ECG platform (e.g., ECG platform 37 described above with respect to FIG. 3B). Mobile device 2540 may have the same components or similar components to those described above with respect to FIG. 3A. For example, mobile device 2540 may run an application (e.g., a local application) and may present mobile interface 2542 on mobile device 2540. Mobile interface 2542 may include, for example, sensing wear information 2544 (for example, a length of time that the user has worn the sensing device), sensing device data 2546, and/or any other relevant information. For example, wear data 2546 may be used to indicate to the user how long they wore the sensing device (for example, smart watch or any other device), which mayincentivize the user to wear the sensing device for longer periods of time to capture more data. Sensing device data 2544 may provide information about data captured by one or more sensing device, such as a plot of the captured data over time, indications of any abnormalities or other conditions, an average of data captured over time, and/or any other information. For example, the data may include heartbeat data, ECG data, and/or any other types of data that may be captured by the sensing device. It is understood that this data may come from one or more sensing device and/or the serving running the ECG platform. For example, the data may be captured by the sensing device, provided to the mobile device 2540, and may be provided by the mobile device 2540 to a remote location for further processing. The health care provider may then analyze the data and provide the analysis to the mobile device 2540 for presentation through the mobile interface 2542. The analysis may be facilitated through the use of any algorithms described herein]

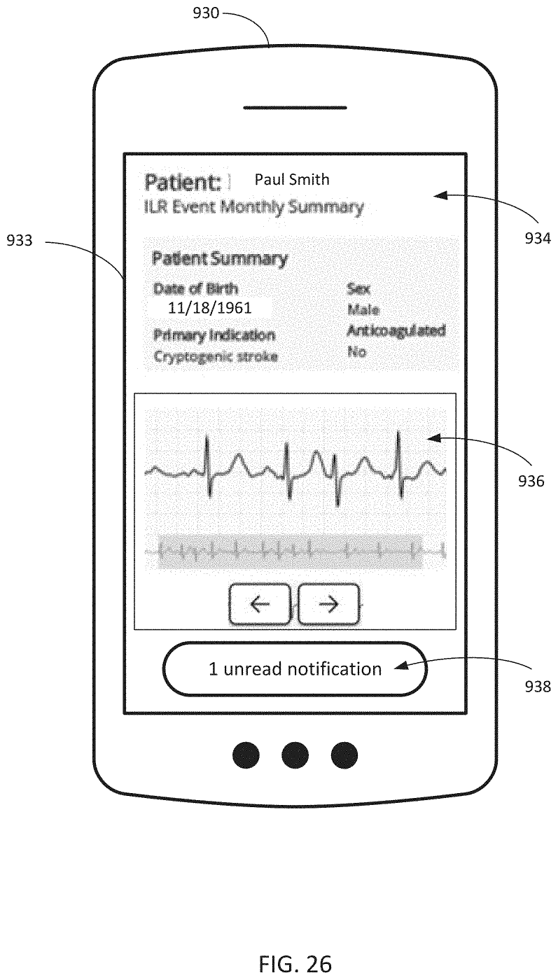

Referring now to FIG. 26, a mobile device presenting a mobile interface is illustrated. Mobile device 930 may be any type of computing device having a processor and a display and in communicate with a server, such as server 922, running an ECG platform (e.g., ECG platform 37 described above with respect to FIG. 3B). Mobile device 930 may have the same components or similar components to those described above with respect to FIG. 3A. For example, mobile device 930 may run an application (e.g., a local application) and may present mobile interface 933 on mobile device 930. Mobile interface 933 may include, for example, patient information 934, ECG information 936, and/or notification information 938.

The server running the ECG platform may communicate all or a portion of mobile interface 933 to mobile device 930. For example, mobile device 930 may communicate patient information 934, ECG information 936, and/or notification information 938 to mobile device 930, which may be presented by the application running on mobile device 930. Alternatively, and/or additionally, certain information presented on mobile interface 933 may be saved locally on mobile device 930. Patient information 934 may include information about the patient (e.g., date of birth, sex, indication, etc.). ECG information 936 may include ECG representation 936 which may be a representation of the ECG signal, such as portion of the signal at a detected ECG event.

ECG information 936 may optionally include information about a detected anomaly, descriptor and/or condition. Notification information 938 may include a notice that the user has a notification or message (e.g., from a health care provider and/or from the ECG platform running on the server). In one example, the notification may be a diagnosis or detected abnormality, condition, and/or anomaly determined by the ECG platform and/or the healthcare provider. Alternatively, or additionally, a notification may include a treatment recommendation Information displayed and provided by the ECG platform may have to be reviewed and/or released by a healthcare professional. Alternatively, the ECG platform may permit the mobile device to display such information once it has been reviewed and/or released by the healthcare professional. It is understood that different data and/or information than that illustrated in FIG. 26 may be presented by mobile interface 933.

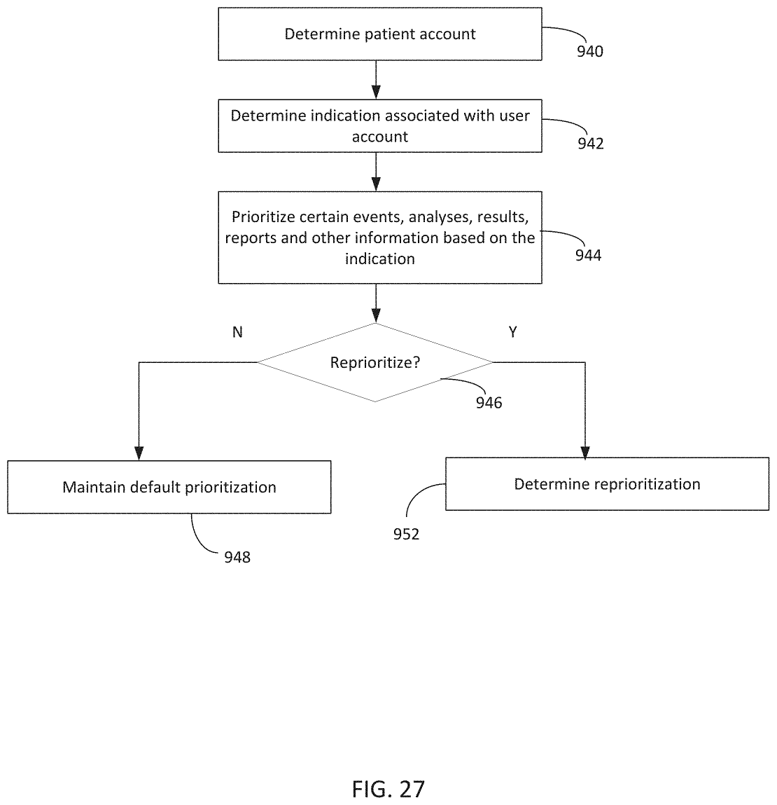

Referring now to FIG. 27, an exemplary process for prioritizing certain information for review by the healthcare provider is illustrated. As there may be many different types of analyses performed on various sensor data, and many different types of results, data and information generated or determined based on the sensed data, it may be useful to prioritize certain results, data, and/or information over others based on known information about the patient, such as an indication relevant to a particular patient. In this manner, the most important data, results, and information for the relevant indication may be presented to the healthcare provider before other less relevant data, results, and information. Some or all of the blocks of the process in FIG. 27 may be performed in a distributed manner across any number of devices (e.g., computing devices and/or servers). Some or all of the operations of the process in FIG. 27 may be optional and may be performed in a different order.

To initiate the process illustrated in FIG. 27 (e.g., on an ECG platform), step 940 may be executed to determine a patient account. For example, a patient name or identification may be used to identify a user account relevant to a specific patient. At step 942, an indication relevant to the patient account may be identified. For example, it may be determined that a particular patient has had a stroke or a heart attack. The patient account may include medical history about that patient and/or medical history about the family of the patient. The indication may be determined from the medical history or otherwise noted in the patient account.

At step 944, the system (e.g., ECG platform) may priority certain events, analyses, results, data, or other information determined by the system based on the indication identified at step 942. For example, results, data and/or other information determined by the system by analyzing sensed data (e.g., ECG data) may be prioritized for review by a healthcare professional. The prioritized data, results, and information may be known by the system to be associated or relevant to the indication. The system may include default settings making such associations between the data, results, identified abnormalities, conditions and/or events and/or information and certain indications.

At decision 946, the system may determine if the events, analyses, data, results, and/or information should be reprioritized. For example, the system may include a reprioritize button on a user interface presenting the events, analyses, data, results and/or information and the healthcare provider may engage the button to indicate that the presentation of the foregoing should be reprioritize or otherwise modified. If the data, results, and/or information should not be reprioritized (e.g., the healthcare provider did not engage the button), then at step 948, the default prioritization should be maintained. Alternatively, input from a user indicating that the data, results, and/or information associated with the indication should be reprioritized (e.g., the button was engaged), then at step 952, the data, results, and/or information prioritized for the indication should be reprioritized. For example, the healthcare provider may manually reprioritize such data, results, and/or information. Prioritization is described further below with respect to FIG. 31A.

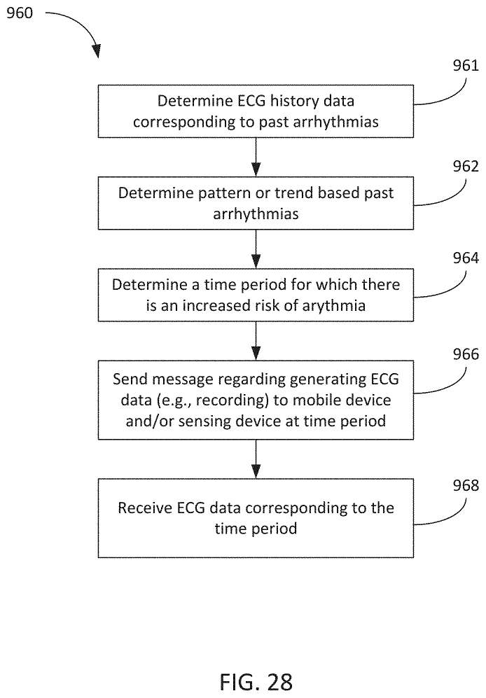

Referring now to FIG. 28, a process for determining a time period for recording ECG data likely to include an arrhythmia event is illustrated. It may be ideal to record ECG data when one or more events occur. However, it may be difficult to predict when such events will occur. Process 960 is an exemplary process for determining a time period for which there is an increased likelihood of an arrhythmia occurring and requesting ECG data corresponding to the time period. Some or all of the blocks of the process in FIG. 28 may be performed in a distributed manner across any number of devices (e.g., computing devices and/or servers). Some or all of the operations of the process in FIG. 28 may be optional and may be performed in a different order.

To initiate the process set forth in FIG. 28 (e.g., on an ECG platform), at step 961 a history of ECG data corresponding to past arrhythmias may be determined. For example, previous events corresponding to arrhythmias may be identified. At step 962, ECG data corresponding to previous events corresponding to arrhythmias may be processed or analyzed to determine a pattern or trend corresponding to the arrhythmias. For example, one or more trained models may be used to detect such patterns and/or trends. At step 964, the patterns and/or trends may be used to determine a time period for which there is an increased risk and/or likelihood of an arrhythmia occurring. The time period may correspond to a time of day, such as between 9:00 am and 9:30 am, for example.

At step 966, a message may be sent to a mobile device and/or to a sensing device to cause the sensing device to generate or obtain ECG data and/or other data relevant to the arrhythmia at the time period. For example, the message may be sent to a mobile device and the mobile device may request such data from the sensing device. Alternatively, the request may be sent directly to the sensing device. In yet another example, a user may need to manually cause the sensing device to record ECG data and the message may instruct the user to start recording the ECG at a certain time and/or for a certain duration. At step 968, the system may receive ECG data and/or other data relevant to the arrhythmia and corresponding to the time period. In this manner, the system and/or mobile device may trigger ECG recordings at times when the patient is likely to experience arrhythmias.

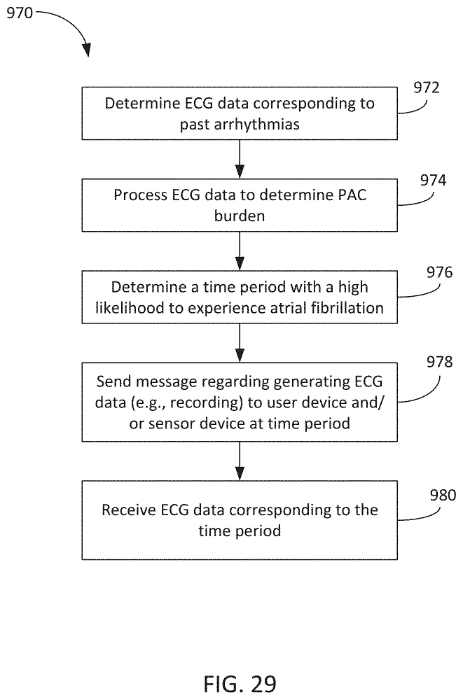

Referring now to FIG. 29, a process for determining a time period (e.g., interval) for recording ECG data likely to include an atrial fibrillation event based on a PAC burden is illustrated. As explained above, it may be difficult to predict when such arrhythmias will occur. Process 970 is an exemplary process for determining an interval for which there is an increased likelihood of an arrhythmia, and specifically atrial fibrillation occurring and requesting ECG data corresponding to the time period. Some or all of the blocks of the process in FIG. 29 may be performed in a distributed manner across any number of devices (e.g., computing devices and/or servers). Some or all of the operations of the process in FIG. 29 may be optional and may be performed in a different order.

To initiate the process set forth in FIG. 29 (e.g., on an ECG platform), at step 972 a history of ECG data corresponding to past arrhythmias may be determined. For example, previous events corresponding to arrhythmias may be identified. At step 974, the previous events corresponding to arrhythmias may be processed or analyzed to determine the total number of premature atrial contractions (PAC) over the total beats in a certain amount of time (i.e., the PAC burden). For example, the techniques described herein may be used to determine PACs in the ECG data and ultimately PAC burden. At step 976 a time period with a high likelihood to experience atrial fibrillation may be determined based on the PAC burden. For example, the techniques described herein may be used generate inferences regarding a likelihood of atrial fibrillation based on the PAC burden.

At step 978, a message may be sent to a mobile device and/or to a sensing device to cause the sensing device to generate or obtain ECG data and/or other data relevant during the time period. For example, the message may be sent to a mobile device and the mobile device may request such data from the sensing device. Alternatively, the request may be sent directly to the sensing device. In yet another example, a user may need to cause the sensing device to record ECG data and the message may instruct the user to start recording the ECG at a certain time. At step 968, the system may receive ECG data and/or other data relevant to the arrhythmia and corresponding to the time period. In this manner, the system and/or mobile device may trigger ECG recordings at times when the patient is likely to experience atrial fibrillation.

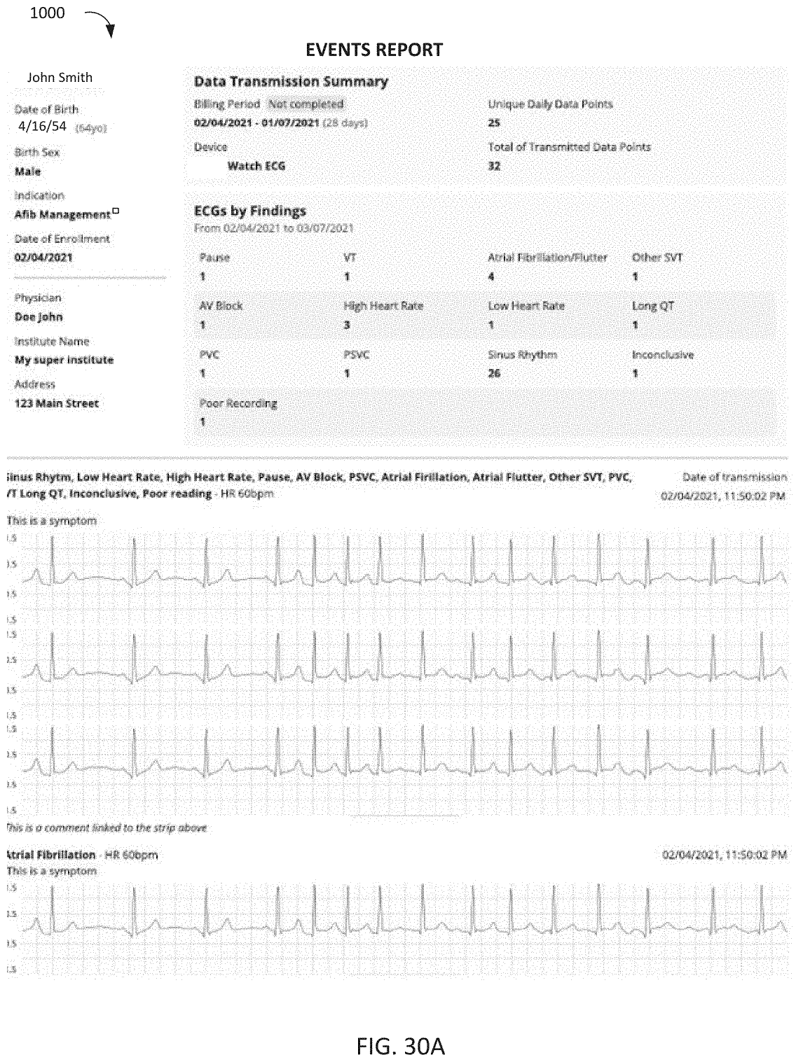

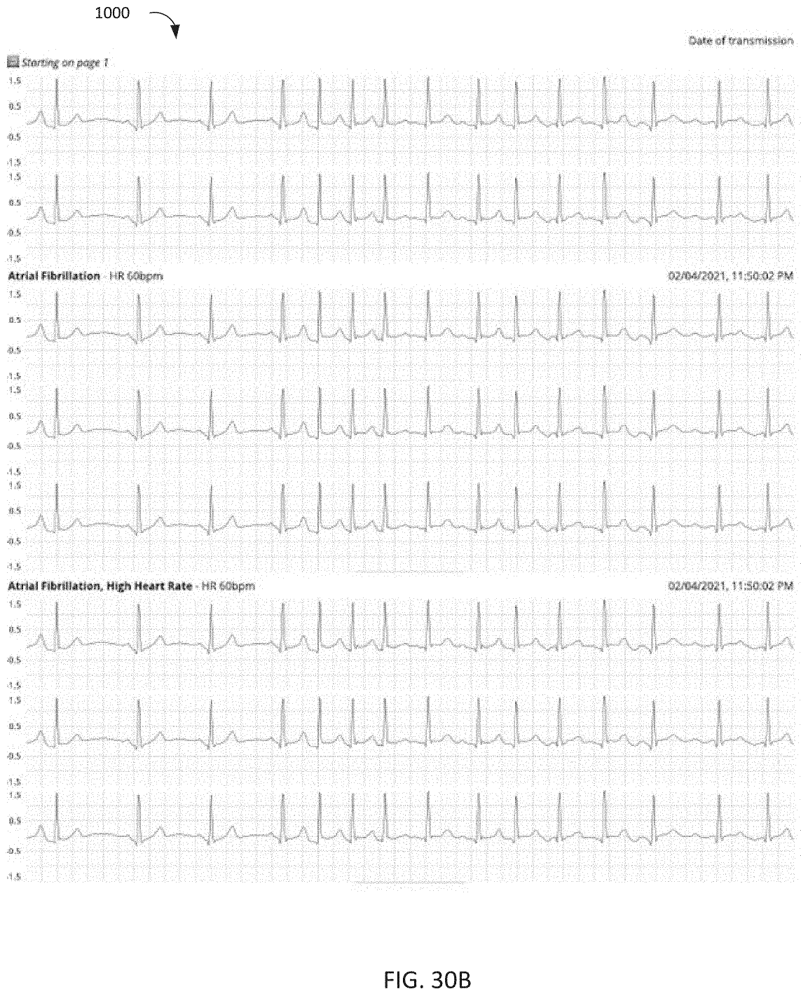

Referring now to FIGS. 30A-30B, an exemplary events report is illustrated. As shown in FIGS. 30A-30B, events report 1000 may include patient information (e.g., name, date of birth, indication, etc.), physician information (e.g., name, institution name, address, etc.), data transmission summary (e.g., device, transmitted data points, billing period, etc.), ECG findings summarizing abnormalities, descriptors, and/or conditions), and one or more ECG representations. For example, portions of ECG strips corresponding to the various abnormalities, descriptors, and/or conditions may be included in events report 1000.

Referring now to FIGS. 31A-31F, various user interfaces are illustrated for displaying patients, indications, classifications, and/or events. It is understood that the user interfaces illustrated in FIGS. 31A-31F may be displayed on any computing device described herein, such as system device 14 described above with respect to FIG. 2.

Referring now to FIG. 31A, patient registration interface 1004 is illustrated. As shown in FIG. 31A, patient registration interface 1004 may include entries for contact information (e.g., email, phone number, etc.), medical history (e.g., indication, medication, etc.) and may permit a healthcare provider to manually prioritize certain criteria (e.g., conditions, descriptors, abnormalities, other information).

Referring now to FIG. 31B, patient list interface 1006 is illustrated. As shown in FIG. 31B, patient list interface 1006 may include a list of patients (e.g., a list of patient's associated with a doctor and/or institution). Patient list interface 1006 may include the patient's name, a date of birth of the patient, an indication associated with the patient, enrollment data, an account status, and/or any other relevant information.

Referring now to FIG. 31C, registration interface 1010 is illustrated. As shown in FIG. 31D, registration interface 1010 may include entries for the patient's name, sex, date of birth, email, phone number, and/or medical history. For example, the medical history entries may include an entry for an indication corresponding to the patient and/or one or more medications taken by the patient.

Referring now to FIG. 31D, registration interface 1012, which may be the same as registration interface 1010, is illustrated. As shown in FIG. 31E, under the “medical history” section of registration interface 1012 there may be indication menu 1014 which may include indications that may be selected. For example, indications may be include Post Atrial Fibrillation ablation, Palpitations, AFib management, and/or none. It is understood that any other indication may be included in indication menu 1014.

Referring now to FIG. 31E, event interface 1018 is illustrated. As shown in FIG. 31C event interface may be accessed from an event list (e.g., by selecting a patient's name). Event interface FIG. 31C may include a patient's name, a classification for the event (e.g., atrial fibrillation), portions of ECG strips corresponding to the event, symptoms, heart rate, and/or any other relevant information. Event interface 1018 may further include a “reclassify” button for reclassifying the event. It is understood that the classification of the event may be determined by the ECG platform and/or may be determined by a sensing device (e.g., sensing device 930 of FIG. 25A).

Referring now to FIG. 31F, event interface 1020, which may be the same as registration interface 1018, is illustrated. As shown in FIG. 31F, event interface may include reclassification menu 1022 next to a classification provided in event interface 1020. For example, reclassification menu 1022 may include several reclassification options such as, sinus rhythm, low heart rate, high heart rate, pause, AV block, PSVC, atrial fibrillation, and/or any other condition, abnormality, and/or descriptor that may classify an event. In this manner, a classification provided by a sensing device (e.g., sensing device 930 of FIG. 25A) may be reclassified by a healthcare provider on using the ECG platform.

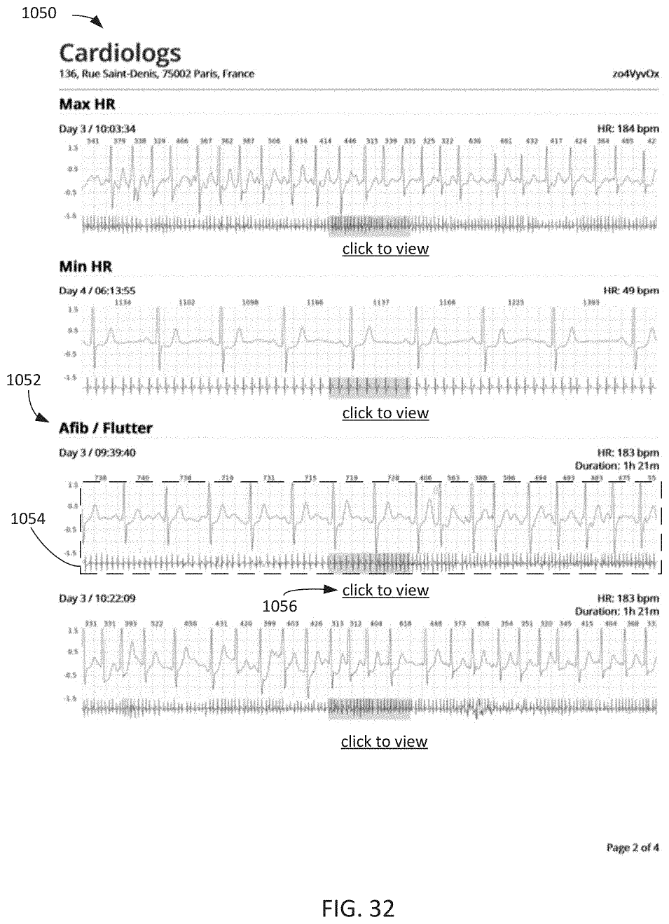

Referring now to FIG. 32, ECG report 1050 is illustrated which may be an exemplary portion of a more comprehensive ECG report such as the report described above with respect to FIGS. 15A-15D. As shown in FIG. 32, ECG report 1050 may include patient information such as the patient's name, primary indication, whether the patient has a pace maker, the patients date of birth, gender and/or a patient ID, and may also include other information such as the overseeing physician, the name of the institute, the date of the analysis and the like. It is understood that ECG report 1050 may be a digital rendering that may be presented on a computing device (e.g., laptop, desktop, tablet, mobile device, etc.) and/or may be a physical print out (e.g., on paper).

As shown in FIG. 32, ECG report 1050 may include various plots (e.g., plot 1052) corresponding to relevant ECG information and/or data. For example, ECG report 1050 may include ECG plots corresponding to maximum heart rate, minimum heart rate, atrial fibrillation, flutter, and/or any other type of ECG, cardiac, physiological and/or biological information. The plots (e.g., plot 1052) may be any type of plot such as an ECG strip, R-R plot, or heart rate density plot, for example. The plots may also indicate, identify or otherwise correspond to a medical condition, event and/or abnormality.

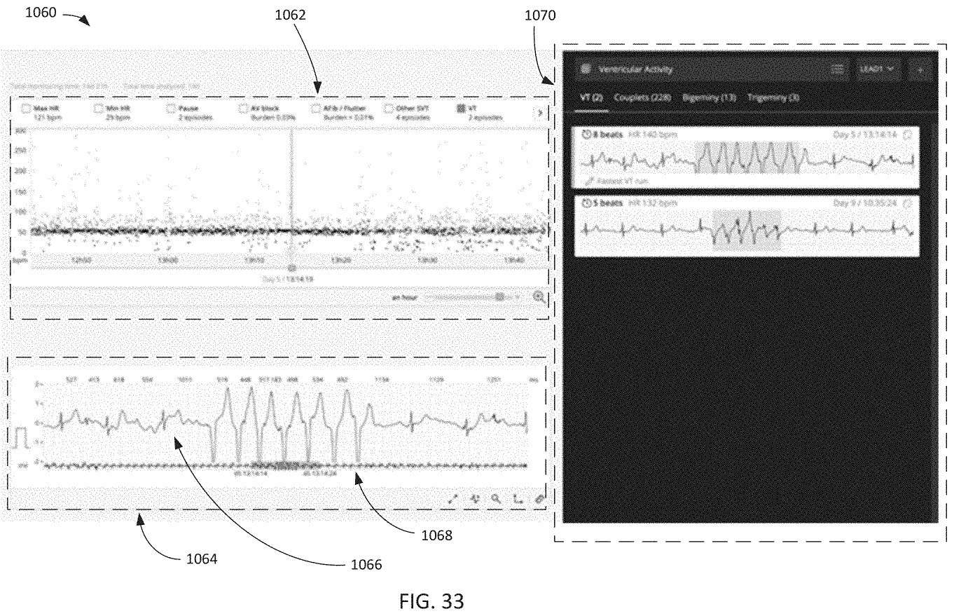

Plot 1052 and/or any other plot in ECG report 1050 may be interactive. For example, plot 1052 may include clickable portion 1054 and/or clickable link 1056, which each may be clicked or otherwise engaged by a user on a computing device. It is understood that clickable link 1056 may be text, an image, an icon, and/or the like. In one example, a physician and/or healthcare provider may receive a digital version of ECG report 1050 and may desire to view more of the signal and/or underlying data in more detail and thus may click clickable portion 1054 of a clickable ECG plot and/or clickable link 1056 using a computing device (e.g., using a touchscreen and/or mouse). Upon clicking clickable portion 1054 and/or clickable link 1056, the user may be redirected to ECG platform 37 and specifically to a viewer version of ECG application 29. For example, the user may be redirected to a viewer application (eg., the viewer application and interface illustrated in FIG. 33). It is understood that ECG report 1050 may include one or more clickable link 1056 and/or clickable portion.

Referring now to FIG. 33, viewer interface 1060 of a viewer application is illustrated. The viewer application may permit a user, such as a user with limited viewing rights (e.g., a limited user), to view additional information corresponding to ECG data and/or other data identified in a report and/or otherwise provide limited access to an ECG platform. For example, the viewer application may generate viewer interface 1060 and may permit a limited user to view the full ECG signal and/or additional ECG data beyond that which was provided in the report. In this manner, the limited user may interact with viewer interface 1060 to view the ECG signals, ECG strips, ECG data, and/or other relevant information. It is further understood that a user with full access to the ECG platform may similarly access viewer application and viewer interface 1060.

As shown in FIG. 33, viewer interface 1060 may be similar to interactive display 101, described above with respect to FIG. 8. For example, viewer interface 1060 may include thee three distinct portions including first portion 1062, which may include a heart rate density plot, second portion 1064 which may include a focused ECG strip 1066 and expanded ECG strip 1068, and third portion 1070 which may include selectable ECG strips organized by identified conditions, events and/or abnormalities.

The heart rate density plot in first portion 1062 may be similar to plot 110 of FIG. 8 and/or may represent the entire signal or a portion thereof and may include selectable identifiers for visually identifying events, conditions and/or abnormalities identified in the ECG signal. Focused ECG strip 1066 may be an ECG strip of a particular timeframe in the heart rate density plot. Focused ECG strip 1066 may correspond to the location along a time axis of an interactive cursor of the heart rate density plot.

Expanded ECG strip 1068 may similarly correspond to a location of the interactive cursor on the on the heart rate density plot and may include an ECG strip having a length of time longer than focused ECG strip 1066 but including the timeframe of the focused ECG strip 1066. Expanded ECG strip 1068 may have a reduced height as compared to focused ECG strip 1066. It is understood that second portion 1064 and first portion 1066 may be linked such that moving the cursor on the heart rate density plot causes the portion of the ECG signal displayed in the focused ECG strip 1066 and the expanded ECG strip 1068 to change based on the location of the cursor on the time axis of the heart rate density plot.

The selectable ECG strips in third portion 1070 may be organized by identified conditions, events, and/or abnormalities. For example, the selectable ECG strips may be organized by ventricular tachycardia (VT), couplets, bigeminy, or trigeminy, for example. Each selectable ECG strip may be selected using the viewer application to view that portion of the ECG signal correspond to the selected ECG strip on first portion 1062 and the second portion 1064. Specifically, the cursor on the heart rate density plot may move to the portion of the heart rate density plot corresponding to the selected ECG strip. Further, focused ECG strip 1066 and expanded ECG strip 1068 will display the selected ECG strip and an expanded version of the selected ECG strip, respectively. In one example, the ECG strips in third portion 1070 may only be those strips included in the ECG report. Alternatively, all identified ECG strips by ECG system may be included in third portion 1070.

Viewer interface 1060 may display greater or fewer plots than that shown in FIG. 33, and/or may display other plots and/or other ECG, biological, physiological and/or any other relevant data. Furthermore, viewer interface 1060 may display comments and/or notes corresponding to the ECG data and/or strips and may optionally permit a limited user to make comments and/or notes. In yet another example, viewer interface 1060 may permit the limited user to provide feedback corresponding to the identified events, conditions and/or abnormalities. For example, the limited user may be able to identify or de-identify an ECG strip as associated with a given condition, event, and/or abnormality. Additionally, and/or alternatively, a limited user may modify and/or revise a report, add comments to a report, add conclusions to a report, and/or sign a report via viewer interface 1060.

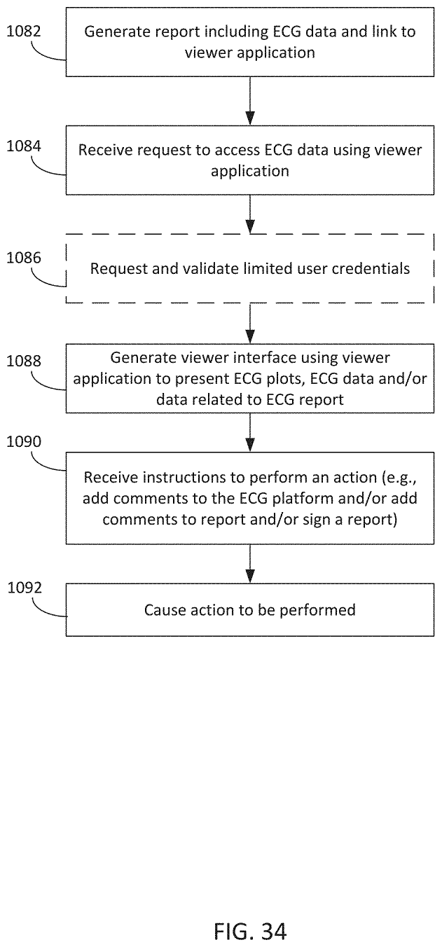

Referring now to FIG. 34, an exemplary process for redirecting a user from the report to the viewer application and viewer interface is depicted. To initiate the process, at block 1082 an ECG system may generate a report as described above with respect to step 68 of FIG. 4 and FIG. 33. At block 1084 an ECG may receive a request to access ECG data using a viewer application, which may be part of the ECG system (e.g., may be an application on the ECG platform). The request to access ECG data may be an automated request or message initiated by an individual viewing a report that has selected a selectable ECG strip and/or selectable link. For example, a healthcare provider may view a digital version of the ECG report on a computing device and may select a selectable ECG strip and/or link to be redirected to the viewer application.

At block 1086, the ECG system, in response to the request to access the viewer application, may request and validate user credentials. For example, the healthcare provider may be a registered limited user of the ECG system and may have a limited user profile with corresponding credentials (e.g., username and passcode). In response to receiving the request to access the viewer application, the ECG system may request the credentials from the limited user and may validate those credentials using the user profile.

At block 1088, the ECG system, via the viewer application, may generate a viewer interface to present ECG plots, ECG data, and/or other data related to the ECG report. For example, the ECG system may generate a viewer interface similar to viewer interface 1062, described above with respect to FIG. 33. At block 1090, the ECG system may receive instructions from user to perform an action (e.g., request to add comments to the ECG platform and/or add comments (e.g., conclusions) to and/or sign an ECG report). For example, a user may use the viewer application to move the user in the heart rate density plot to view various portions of the ECG signal, may select a selectable ECG strip for viewing, may request to add comments corresponding to an ECG strip, and/or may request to comment on and/or sign an ECG report. At block 1092, the ECG system may cause the action to be performed on the viewer application (e.g., sign report, add comments to report, add/or comments to ECG platform) based on the received instructions.

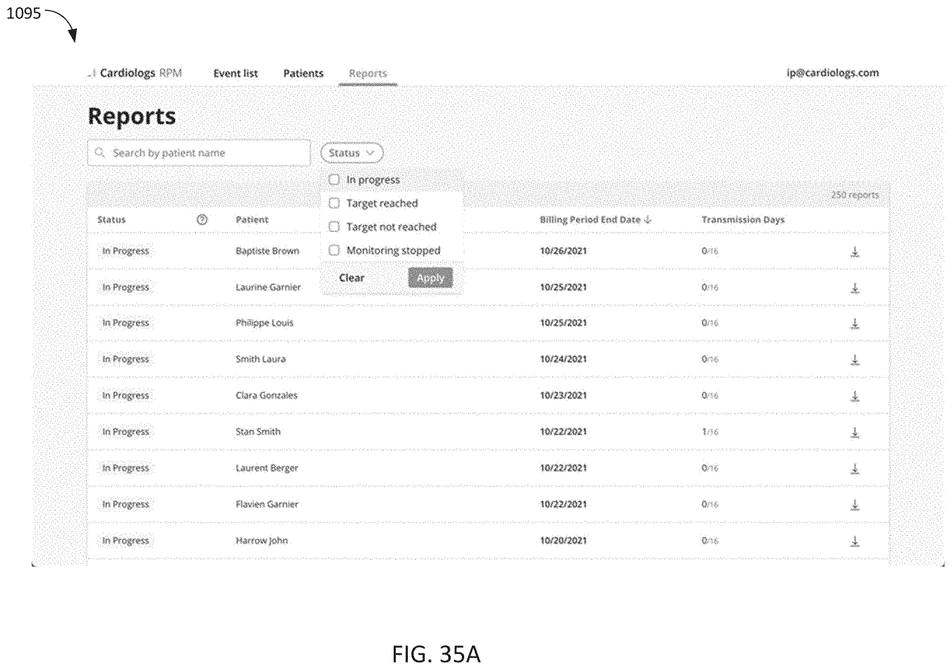

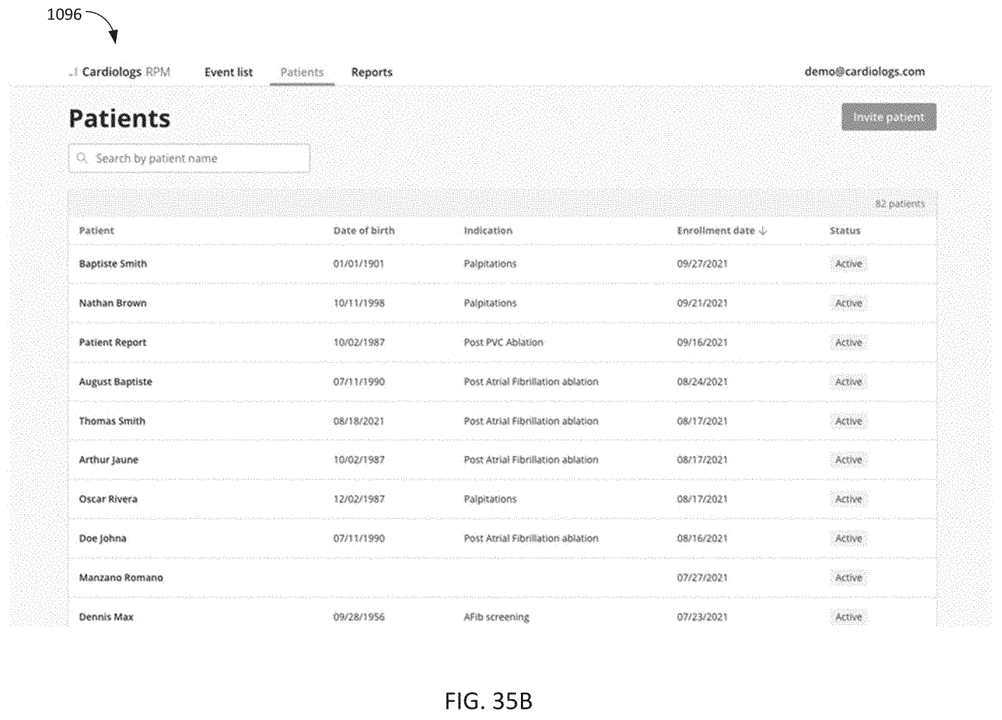

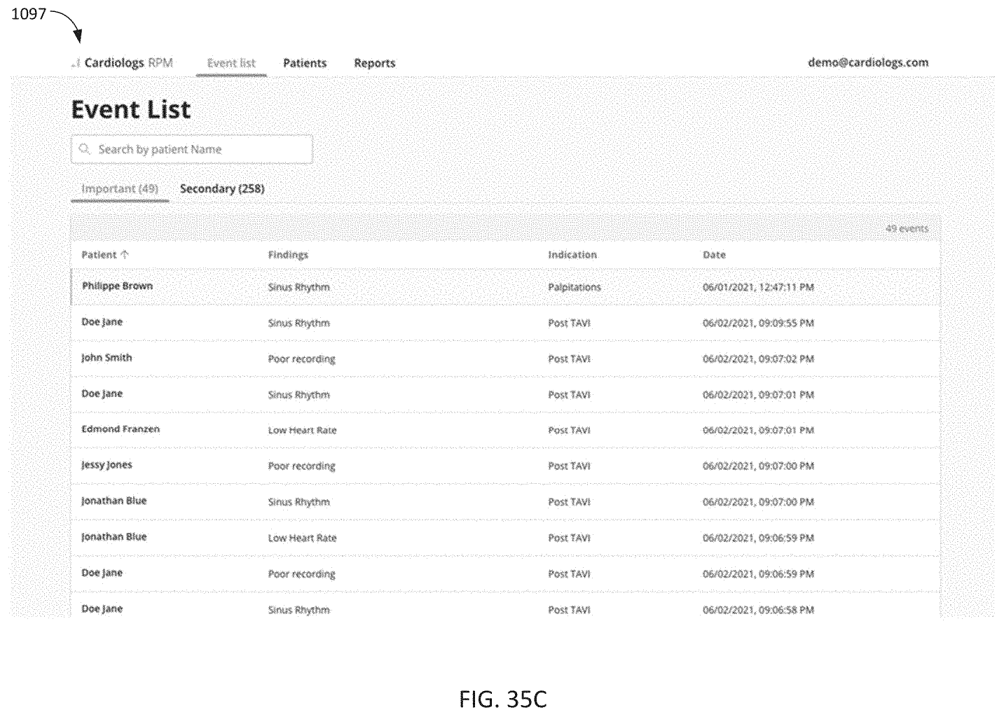

Referring now to FIGS. 35A-35C, report, patients and event list interfaces are illustrated. As shown in FIG. 35A, report interface 1095 may provide a status for reports in the ECG system. For example, report interface 1095 may include a column for the patient's name, the status of the report (e.g., in progress, target reached, target not reached, monitoring stopped), billing period end date, and/or transmission days. Further, report interface 1095 may include a search field (e.g., for the patient name) and a status filter (e.g., filter by in progress).

As shown in FIG. 35B, patient interface 1096 may include relevant information for patients in the ECG system. Patient interface 1096 may include a column for the patient's name, date of birth, indication, enrollment date, and/or status (e.g., active). Patient interface 1096 may include a search field (e.g., by patient name) and/or may be filtered.

As shown in FIG. 35C, event list interface 1097 may include relevant events for patients. For example, event list interface 1097 may include tabs for important and/or second secondary events and under each tab may include a column for patient name, findings (e.g., sinus rhythm, low heart rate, etc.) indication (e.g., palpitations) and/or date. Event list interface 1097 may include a search field (e.g., by patient name) and/or may be filtered.

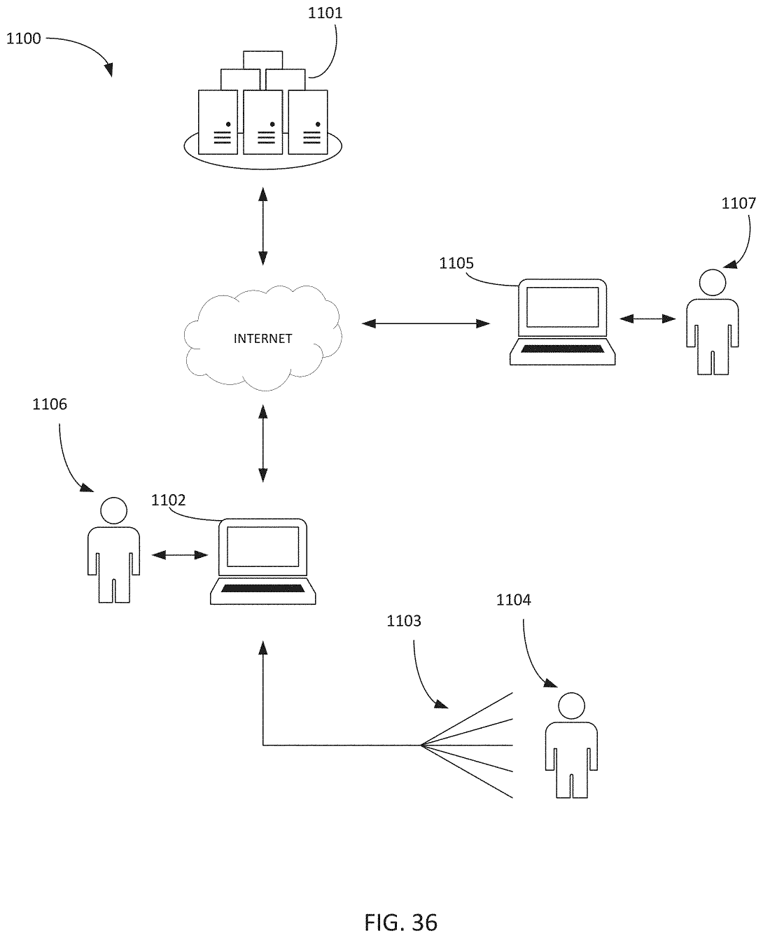

FIG. 36 is a diagram illustrating an example system 1100. System 1100 may include a first system device 1102 associated with an ECG sensing device 1103, a second system device 1105, and/or one or more server(s) 1101. First system device 1102 may be operated by one or more user(s) 1106, ECG sensing device 1103 may capture ECG data relating to one or more user(s) 1104, and second system device 1105 may be associated with one or more user(s) 1107. It should be noted that any reference made herein to a single element (for example, a first system device 1102, an ECG sensing device 1103, a second system device 1105, a server 1101, a user 1106, a user 1104, a user 1107, or the like) is not intended to be limiting and the same descriptions may also be applicable to any number of these elements and/or any other element as well. Additionally, any refer to an “analysis” is not intended to be limiting and the analysis may comprise multiple types of analyses as well.

Server 1101, first system device 1102, and/or second system device 1105 may include any suitable processor-driven device including, but not limited to, a mobile device or a non-mobile, e.g., a static device., a personal computer (PC), a wearable wireless device (e.g., bracelet, watch, glasses, ring, etc.), a desktop computer, a mobile computer, a laptop computer, an Ultrabook™ computer, a notebook computer, a tablet computer, a server computer, a handheld computer, a handheld device, an internet of things (IoT) device, a sensor device, or any other well-known computing device.

First system device 1102 may receive and/or otherwise obtain data (such as ECG data captured by an ECG sensing device 1103) associated with user 1104 and send the data to server 1101 for storage purposes. User 1107 may be provided permissions to access the data through server 1101 (for example, using second system device 1105). User 1107 may provide an indication to server 1101 (for example, using second system device 1105) to analyze the ECG data (that is any analyses may be performed by server 1101 at the direction of user 1107 through second system device 1105). Server 1101 may then perform an analysis on the ECG data and may generate a report based on the analysis (e.g., based on a command to generate a report). This report may then be provided back to first system device 1102. Additionally and/or alternatively, the report may be maintained on server 1101 for access by partner user 1106 and/or third party user 1107. In one example, first system device 1102 may be a pharmacy that may obtain ECG data from a patient and ultimately provide a report to the same patient. In this example, user 1107 may be a physician and/or healthcare provider that may review the analysis performed by server 1101.

ECG sensing device 1103 may be the same or similar to ECG sensing device 13 as described above with respect to FIG. 2. For example, sensing device 1103 may be one or more electrodes that may be disposed on one or more leads. Sensing device 1103 may be in electrical communication with first system device 1102 running an ECG application (for example, ECG application 29 and/or any other ECG application) such that the electrical signal sensed by sensing device 1103 may be received by the ECG application (running on the first system device 1102, for example). The ECG application may include instructions that cause sensing device 1103 to sense or otherwise obtain ECG data.

First system device 1102 is preferably one or more computing devices (e.g., laptop, desktop, tablet, smartphone, smartwatch, etc.) having the components described below with reference to FIG. 3A and the functionality described herein. System device 1102 may be running the ECG application may connect with server 1101 running (for example, ECG platform 37) via any well-known wired or wireless connection. For example, system device 1102 may connect to the Internet using well-known technology (e.g., Wi-Fi, cellular, cable/coaxial, and/or DSL) and may communicate with server 1101 over the Internet. Any of the other elements of the system 1100 may also communicate with one another using any well-known wired or wireless connection as well.

First system device 1102 may be associated with partner user 1106. In some embodiments, partner user 1106 may be a user that may be provided limited permissions with respect to server 1101. For example, partner user 1106 may only upload recordings, request report generation, and/or modify administrator information (for example, one or more of information for one or more administrative entities, healthcare entities, healthcare provider information, patient information, patient demographics, and the like), among other types of functions. Unlike other users that may access server 1101 for example, user 1107 and/or any other user), partner user 1106 may not be able to access any analyses stored within server 1101. These are merely examples of a manner in which a partner user 1106 may be restricted and are not intended to be limiting in any way.

Server 1101 is preferably one or more servers having the components described below with reference to FIG. 3B (and/or any other components described herein) and the functionality described herein. Server 1101 may be the same or similar to server 15 as described above with respect to FIG. 2. Server 1101 may have processing power superior to first system device 1102 such that server 1101 can process and analyze (for example analyses described with respect to FIG. 4 and/or any other types of analyses described herein) any ECG data received from first system device 1102 and/or any other data. Server 1101 may also be configured to produce reports based on any analyses of any ECG data. As will be readily apparent to one skilled in the art, server 1101 may include a plurality of servers located in a common physical location or in different physical locations. In one example, server 1101 may be located in a different, remote location (e.g., on the cloud) than first system device 1102, although server 1101 and first system device 1102 may be located in a common location (e.g., on a local area network (LAN)).

Server 1101 may also include the capability to provide storage for ECG data, analysis data, reports generated relating to ECG data, and/or any other types of data. Server 1101 may be associated with one or more user interface(s) that may be presented to a user (such as user 1106 and/or user 1107) through first system device 1102, second system device 1105, and/or any other system device. A user interface may allow a user to view any data stored to server 1101 and/or perform any other types of functions. For example, user 1106 (through second system device 1102) may be able to upload ECG data received from ECG sensing device 1103 to server 1101. User 1106 may provide access to the data to user 1107, such that user 1107 may then be provided the ability to indicate to server 1101 to perform an analysis on any uploaded data. That is, the analyses may not necessarily be performed by user 1107 and/or second system device 1105, but rather user 1107 may have control over when server 1101 performs any analyses of the ECG data.

Particularly, server 1101 may include one or more folders that may be presented through the user interface and that may store various ECG data, patient data, medical data, or the like. Folders may only be accessible by users who are granted permission to access the folders. In this manner, a first entity may have a number of folders stored on server 1101 (for example, including ECG data for different patients). The first entity may desire for a second entity to access one of the folders to obtain ECG information for a particular patient for analysis, but may not desire for the second entity to be able to access any of the other folders. Server 1101 may thus provide the capability for permissions to be established such that the second entity is only able to access the desired folder, but not any of the remaining folders associated with the first entity.

An example use case may include the following. User 1106 (for example, through the first system device 1102) may upload a recording of ECG data to server 1101 into a folder specially dedicated to user 1107. The folder can be configured so that user 1107 may share patient data (name, date of birth, etc.) with the second system device 1105. User 1107 may only see the recordings that are in this particular folder, rather than being able to access all of the recordings associated with the first system device 1102. The user 1107 and/or the third-party system device 1105 may provide an indication to server 1101 to perform an analysis on the ECG data (for example, using any of the analyses described herein) and/or generate a report. The report may be based on the analyses generated by the server 1101 (and may optionally be saved into the folder from which the records were obtained and/or any other folder). In one example, the report may be communicated or otherwise accessed by user 1106.

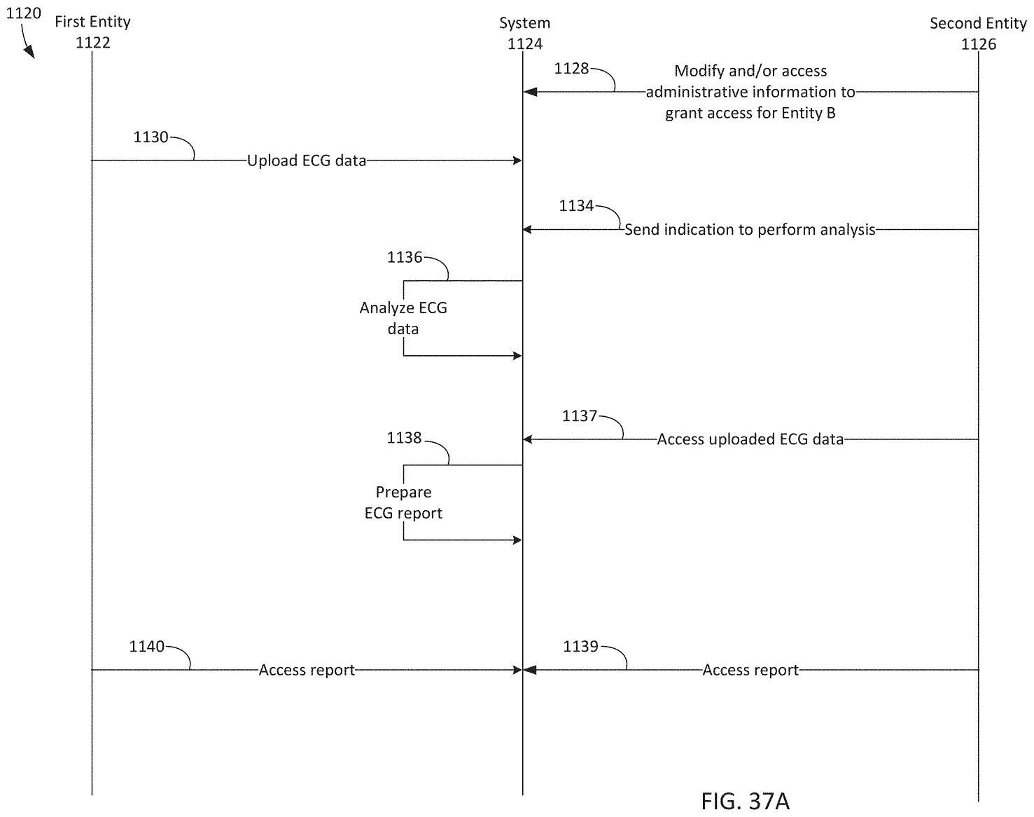

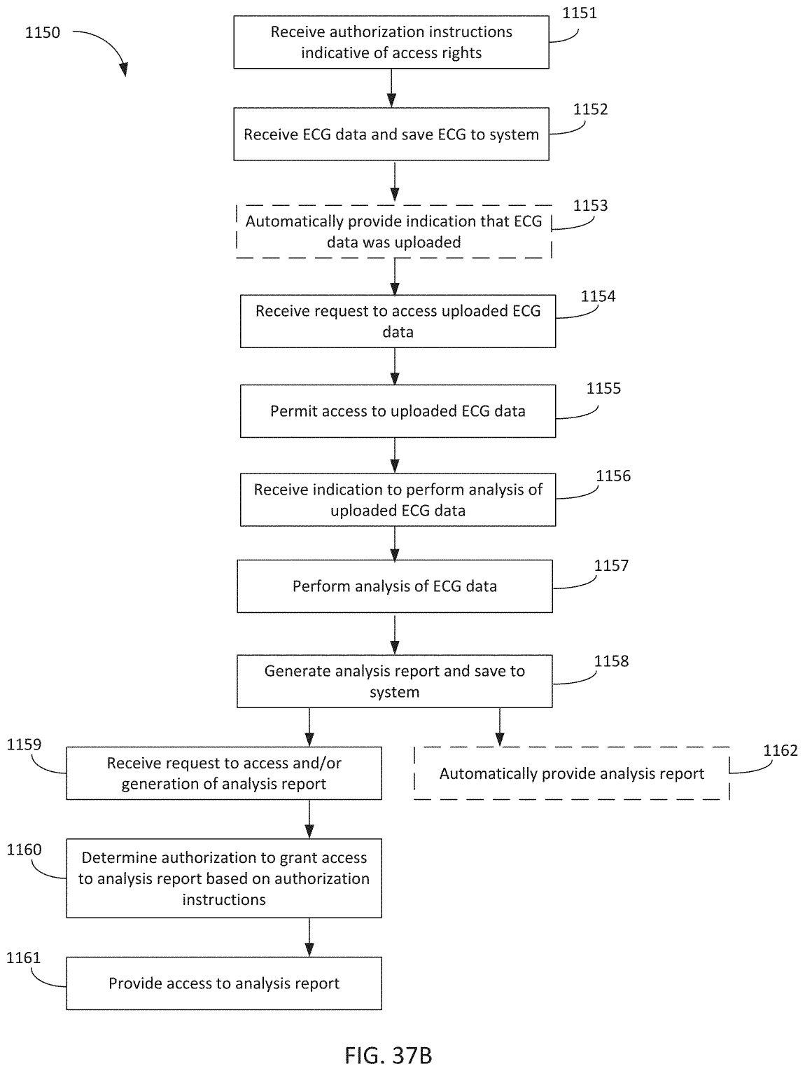

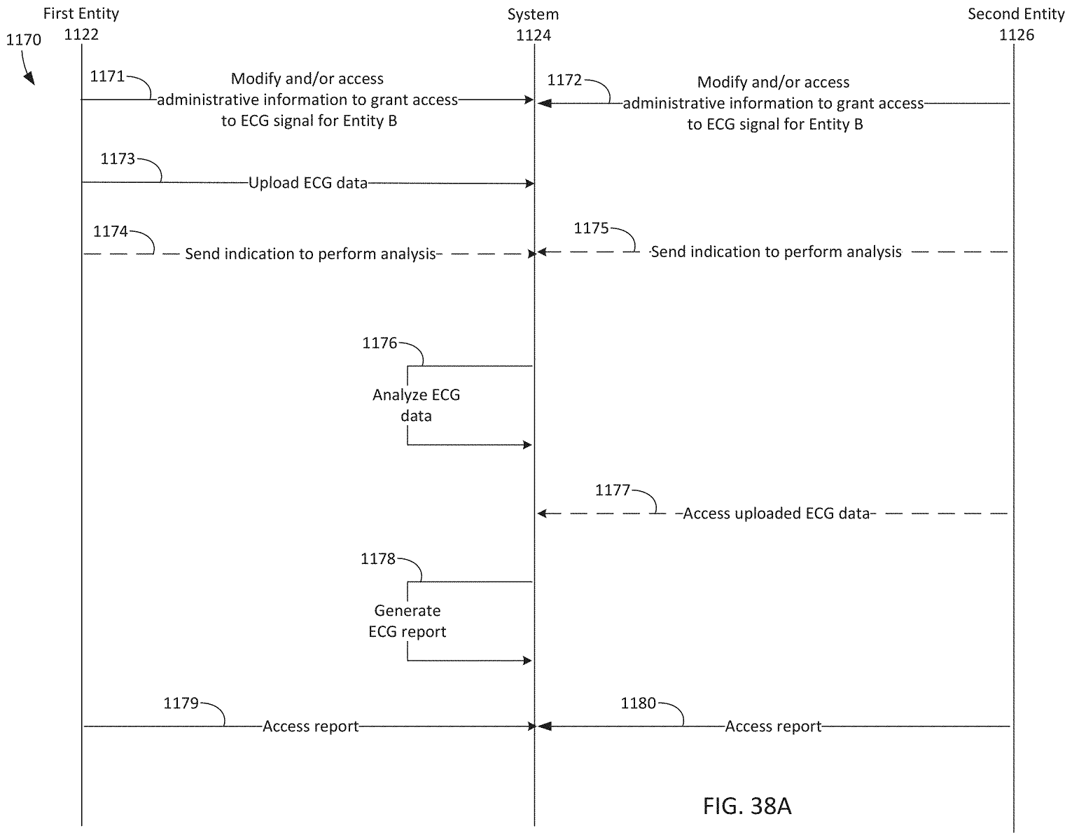

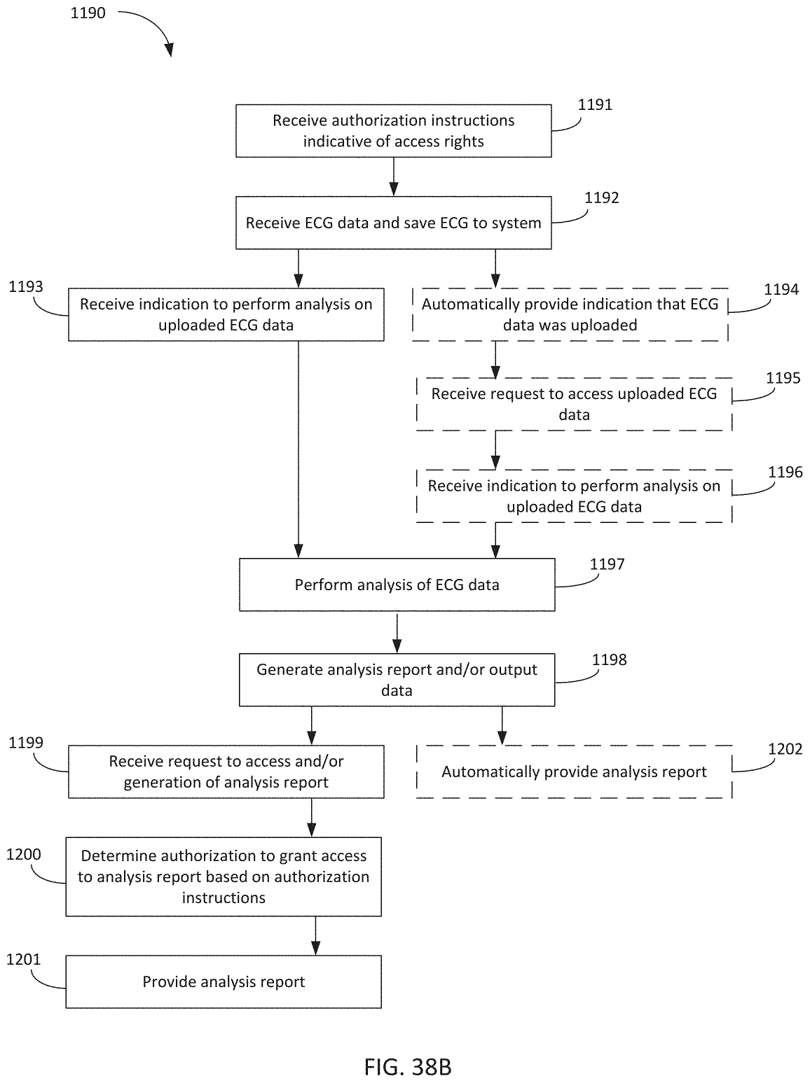

Referring now to FIG. 37A, an exemplary process 1120 for performing ECG analyses and/or reports and restricting access thereto is depicted. Process 1120 may illustrate operations that may be performed in association with entities that produce ECG data but may not analyze the ECG data themselves (for example, first system device 1102 of FIG. 36 may be first entity 1122 in FIGS. 37-38). That is, the data may be outsourced to a third-party or other entity to manage or otherwise oversee the analysis (for example, second system device 1105 of FIG. 36 may be second entity 1126 in FIGS. 37-38). At a high-level, first entity 1122 may produce or obtain the ECG data and upload the data to remote system 1124 (which may be server 1101 of FIG. 36, for example). Second entity 1126 may then access the uploaded ECG data, may indicate (e.g., instruct and/or command) to system 1124 to analyze the data and system 1124 may then generate a report based on any analyses that are performed. The report may then be accessed by first entity 1122 and/or second entity 1126. In some scenarios, system 1124 may automatically provide the report to first entity 1122 upon generation and/or may provide an indication to first entity 1122 that the report was generated. Additionally, or alternatively, an indication that ECG data was uploaded may automatically be provided to second entity 1126.

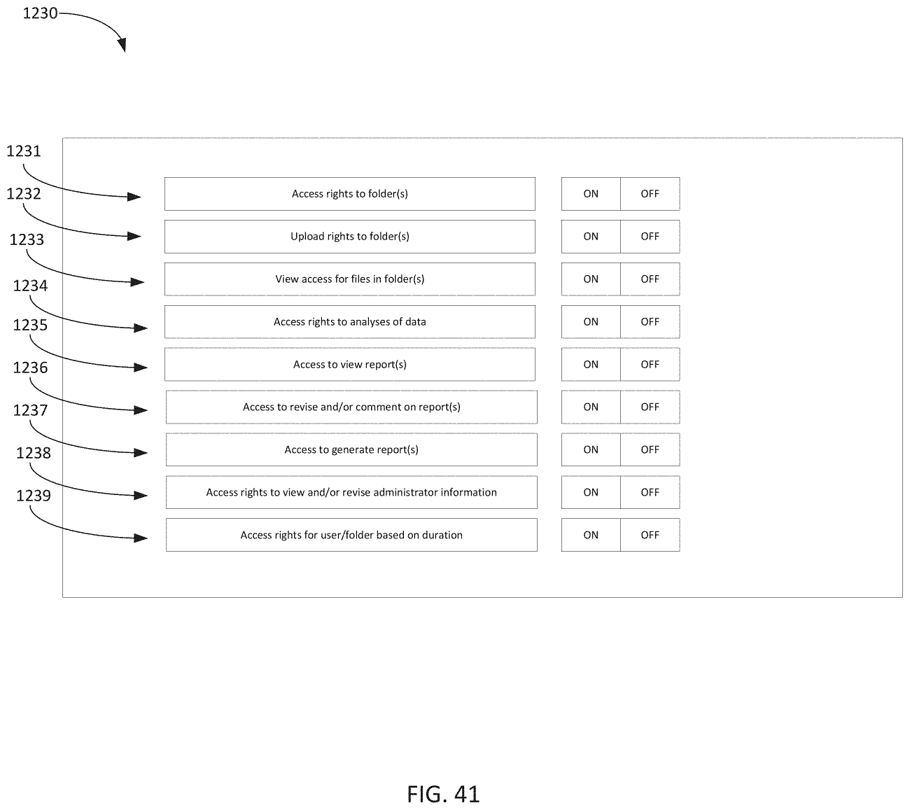

Turning to process 1120, at operation 1128, computer-executable instructions stored on a memory of a device, such as a computing device, may be executed to modify and/or grant access to various folders, data and/or functionality on system 1124 for the first entity 1122 (e.g., as described below with respect to FIG. 41). Likewise, system 1124 (for example, server 1101 and/or any other system) may receive an indication of a modification or granting of access from second entity 1126. For example, second entity 1126 may have the capability to indicate, using system 1124, which users are able to upload data and/or perform certain actions with respect to any of the data uploaded to the system 1124 by first entity 1122. Any other types of user rights may also be indicated. For example, second entity 1126 may have a number of folders included within system 1124. Second entity 1126 may grant first entity 1122 access to a folder associated with second entity 1126. However, first entity 1122 may not have permission to access other data on system 1124. Similarly, first entity 1122 may also have the capability to indicate, using system 1124, which users are able to upload data and/or perform certain actions with respect to any of the data uploaded to the system 1124 by first entity 1122. Additional examples of user rights may be presented FIG. 41.

At operation 1130, computer-executable instructions stored on a memory of a device, such as a computing device, may be executed to upload ECG data. For example, first entity 1122 may capture and/or obtain ECG data from a user (for example, user 1104 in FIG. 36) and may upload the captured data to system 1124. Likewise, system 1124 may receive the uploaded data from first entity 1122. The data may, for example, be uploaded into a first folder that first entity 1122 has permission to access. These permissions may be established by second entity 1126 (for example, as described with respect to operation 1128) such that second entity 1126 may obtain the uploaded data for analysis purposes. However, it should be noted that the location in which the data is uploaded may also be managed by first entity 1122 instead of second entity 1126. That is, first entity 1122 may upload data into a location within system 1124 that second entity 1126 is authorized to access based on permissions granted by first entity 1122.

At operation 1134, computer-executable instructions stored on a memory of a device, such as a computing device, may be executed to send an indication to perform an analysis. For example, second entity 1126 may provide an indication to system 1124 to perform an analysis of the uploaded ECG data. Such an indication may be provided, for example, through a user interface associated with system 1124. Likewise, system 1124 may receive an indication to perform an analysis from the ECG data from the second entity 1126. It is understood that operation 1134 may be optional and that system 1124 may automatically initiate operation 1136.

At operation 1136, computer-executable instructions stored on a memory of a device, such as a computing device, may be executed to analyze the ECG data. For example, the ECG data may be analyzed by system 1124 in accordance with the ECG processing system 10 described with respect to FIG. 4 and/or may be analyzed based on any other type of analysis using any other system and/or device mentioned herein.

At operation 1137, computer-executable instructions stored on a memory of a device, such as a computing device, may be executed to access the uploaded ECG data and/or analyses of ECG data. Likewise, system 1124 may provide access to second entity 1126 to the uploaded ECG data and/or analyses of ECG data.

At operation 1138, computer-executable instructions stored on a memory of a device, such as a computing device, may be executed to generate a report based on the analysis of the ECG data performed in block 1134. The report may include any information relevant to the analysis of the ECG data. For example, the report may indicate whether any anomalies exist in the patient's ECG data. The report may be generated by system 1124. In one example, system 1124 may generate a report in response to second entity 1126 and/or first entity 1122. Alternatively, system 1124 may automatically generate a report.

At operation 1139, computer-executable instructions stored on a memory of a device, such as a computing device, may be executed to access the report. That is, second entity 1126 may access the report through server 1101 (for example, through a user interface). Additionally, an indication that the report was generated may automatically be provided to second entity 1126 and/or the report itself may automatically be provided to second entity 1126.

At operation 1140, computer-executable instructions stored on a memory of a device, such as a computing device, may be executed to access the report. That is, if first entity 1122 has rights to access the report, first entity 1122 may access the report through server 1101 (for example, through a user interface). Additionally, an indication that the report was generated may automatically be provided to first entity 1122 and/or the report itself may automatically be provided to first entity 1122.

Referring now to FIG. 37B, an exemplary process 1150 for performing ECG analyses and restricting access and/or rights to an ECG platform is depicted. Some or all of the blocks of the process flows in this disclosure may be performed in a distributed manner across any number of devices (e.g., servers, computing devices, user devices and/or servers). Some or all of the operations of the process flow may be optional and may be performed in a different order. It is understood that the process in FIG. 37B may be performed by a server (e.g., server 1101 of FIG. 36) and may be directed to a process by which a third-party (for example, second entity 1126 of FIG. 37A) or other entity facilitates the analysis of any ECG data (and/or other types of data) that is uploaded or otherwise provided to the server by a provider (for example, first entity 1122 of FIG. 37A).

At operation 1151, computer-executable instructions stored on a memory of a device, such as a computing device, may be executed to receive an indication of a modification or granting of access rights. For example, a second entity may have the ability to indicate, using an ECG system, which users are able to perform certain actions with respect to any of the data uploaded to the system by a first entity. Any other types of user rights may also be indicated. For example, the second entity may have a particular portion (e.g., one or more folders) of the system that the second entity may use to store data and/or that any other entity may use to store data based on permissions provided by the second entity. This portion may be represented by a number of folders presented through a user interface associated with the system, for example. However, the storage may be depicted in any other format as well (i.e., other than folders). In this manner, it should be noted that any reference to a “folder” herein may simply indicate a location within the system that is reserved for storage by second entity and any other entities that have permission to use this storage space. The second entity may grant first entity access to a folder including any data in such folder. However, the first entity may not have permission to access other data uploaded to the system (e.g., in different folders). Additional examples of user rights may be presented FIG. 41. The second entity may have any number of different folders included within the system that may be intended for any number of different entities. For example, the second entity may facilitate analyses of ECG data received from any number of different entities. A first folder may be reserved for the first entity, a second folder may be reserved for a third entity, etc. This may allow each of the entities to upload ECG data to their respective folders within the system to allow the second entity to access the data, while preventing the third-party entity or other entity from accessing the data uploaded by the first entity, and vice versa.

In some embodiments, the data may also be uploaded to one or more folders associated with first entity instead of second entity. That is, first entity may manage the one or more folders as an administrator and may indicate permissions to access the folder. This may provide administrative control to first entity that is uploading the ECG data rather than second entity that is facilitating the analysis of the ECG data.

At operation 1152, computer-executable instructions stored on a memory of a device, such as a computing device, may be executed to receive uploaded ECG data. For example, the first entity may capture and/or obtain ECG data from a user (for example, user 1104 in FIG. 36) and may upload the captured data to the system. Particularly, the first entity may be associated with a first system device, which may receive data captured from the user by an ECG sensing device. The system may receive this uploaded data from the first entity. The data may, for example, be uploaded into a first folder that the first entity has permission to access. These permissions may be established by the first entity and/or by the second entity (for example, as described with respect to operation 1151). The location in which the data is saved may be based on authorization instructions and/or permissions established by the first entity or the second entity.

At optional operation 1153, computer-executable instructions stored on a memory of a device, such as a computing device, may be executed to automatically provide an indication that the ECG data was uploaded. That is, system may be configured to automatically provide a notification to the second entity when new data is uploaded into a folder that the second entity has permissions to access. In some embodiments, these automatic notifications may be configured in settings associated with the system by the first entity and/or the second entity. For example, the second entity may prefer to receive notifications so that the second entity may have knowledge of when new data is uploaded for which second entity may facilitate an analysis through the system (for example, when ECG data produced by first entity is outsourced to the second entity or any other entity to handle the analysis as aforementioned). The automatic notifications may be provided in any suitable manner, such as a phone call, email communication, text message, and/or the like. The automatic notifications may also be provided within the system (for example, presented through a user interface) and/or through the use of an application programming interface (API).

At operation 1154, computer-executable instructions stored on a memory of a device, such as a computing device, may be executed to receive a request to access the uploaded ECG data. For example, the uploaded data may be stored within the system, and the second entity may need to access the data in order to facilitate the analysis. The second entity may access this data, for example, through a user interface associated with system that may be presented to the second entity in any suitable manner (for example, a user interface associated with a website, a mobile device application, a desktop software application, and/or any other form in which a user interface may be presented). The second entity may access a location in which the data is stored within the system using the user interface. The system may confirm that the second entity is authorized to view the data and/or any representations thereof (for example, based on the authorization instructions provided by the first entity). If it is determined that the second entity is authorized to view the data and/or any representations thereof, then the data may be presented to the second entity through the user interface. In one example, the second entity may not need to manually perform an action to initiate this request. Rather, from the perspective of the second entity, it may appear that automatic access to the data is provided.

At operation 1155, computer-executable instructions stored on a memory of a device, such as a computing device, may be executed to permit access to the uploaded ECG data.

At operation 1156, computer-executable instructions stored on a memory of a device, such as a computing device, may be executed to receive an indication to perform an analysis of the uploaded ECG data. For example, the second entity may provide an indication to the system to perform an analysis of the uploaded ECG data. Such an indication may be provided, for example, through a user interface associated with the system. Alternatively, the system may automatically perform analysis of ECG data uploaded to the ECG system and/or saved in a certain folder. Alternatively, the analysis may automatically be performed by the system without requiring the indication.

At operation 1157, computer-executable instructions stored on a memory of a device, such as a computing device, may be executed to perform the analysis of the uploaded ECG data. For example, the analysis may include any processes described with respect to FIG. 4 and/or any other types of possible analyses of ECG data described herein. Such analyses, for example, may be used to identify abnormalities and conditions of a patient. Abnormalities and conditions may include but are not limited to, sinoatrial block, paralysis or arrest, atrial fibrillation, atrial flutter, atrial tachycardia, junctional tachycardia, supraventricular tachycardia, sinus tachycardia, ventricular tachycardia, pacemaker, premature ventricular complex, premature atrial complex, first degree atrio-ventricular block (AVB), 2nd degree AVB Mobitz I, 2nd degree AVB Mobitz II, 3rd degree AVB, Wolff-Parkinson-White syndrome, left bundle branch block, right bundle branch block, intraventricular conduction delay, left ventricular hypertrophy, right ventricular hypertrophy, acute myocardial infarction, old myocardial infarction, ischemia, hyperkalemia, hypokalemia, brugada, and/or long QTc.

At operation 1158, computer-executable instructions stored on a memory of a device, such as a computing device, may be executed to generate an analysis report and/or output data corresponding to analysis of the uploaded ECG data. The report and/or output data may include any information relevant to the analysis of the ECG data. For example, the report and/or output data may indicate whether any anomalies, conditions, and/or events exist in the patient's ECG data. The report and/or output data may also include any other types of information, such as an illustration of the ECG data, an indication of different points of interest in the data, any data classifications that were performed, and/or any other information that may be relevant to the analysis. The report and/or output data may be automatically generated by the system or may be generated at the instruction of the first entity or the second entity. The report and/or output data may also be stored on system, such that it may be accessed at a later time (for example, accessed by first entity). In some cases, the report and/or output data may be stored in the same folder including the uploaded ECG data. However, the report and/or output data may also be stored in any other location as well. The location in which the report is saved may be based on the aforementioned authorization instructions. That is, the report may be saved within a folder that is accessible by the second entity and/or any other entity. Alternatively, the report may not be saved on the system and may only be generated and communicated to a device upon request.

At operation 1159, computer-executable instructions stored on a memory of a device, such as a computing device, may be executed to receive a request to access the output data and/or analysis report based on the output data (e.g., from the first entity and/or the second entity). The request to access the analysis report may include a request to generate the analysis report. At operation 1159, computer-executable instructions stored on a memory of a device, such as a computing device, may be executed to determine authorization to grant access to and/or generate the analysis report based on authorization instructions. For example, the system may determine if the first entity or the second entity has authorization to access the location within the system in which the report is stored. A further authorization may also be required to access the report within the location. That is, even if the first entity or second entity have access to the location itself, the first entity or second entity may require additional authorization to view the report as well.

At operation 1160, computer-executable instructions stored on a memory of a device, such as a computing device, may be executed to determine authorization to grant access to and/or generate the analysis report based on authorization instructions.

At operation 1161, computer-executable instructions stored on a memory of a device, such as a computing device, may be executed to provide the analysis report (e.g., to the first entity and/or second entity). For example, the first entity may be able to view the report through a user interface associated with the system. The system may also allow the first entity to download the report to a local device for local storage and/or viewing. However, in some cases, system may prevent any users from downloading the report, and rather may maintain the report within system for viewing.

At optional operation 1162, computer-executable instructions stored on a memory of a device, such as a computing device, may be executed to automatically provide the analysis report (e.g., to the first entity and/or the second entity). For example, similar to the optional automatic notifications provided to second entity when data is uploaded, automatic notifications may also be provided to first entity when an analysis report is generated. These automatic notifications may be configured in settings associated with the system by the first entity and/or the second entity (and/or may not require any configuration by first entity and/or the second entity). These automatic notifications may allow first entity to be notified when an analysis has been completed and a report has been generated without having to access the system to check to determine if the report has been generated. The automatic notifications may also be provided in any suitable manner, such as a phone call, email communication, text message, and/or the like. The automatic notifications may also be provided within the system (for example, presented through a user interface) and/or through the use of an application programming interface (API).

Referring now to FIG. 38A, an exemplary process 1170 for performing ECG analyses and/or reports and restricting access thereto is depicted. The process 1170 may illustrate operations that may be performed for entities that produce ECG data and interact with system 1124 themselves to initiate an analysis of the ECG data to produce a report, but may occasionally outsource the analysis initiation to a third-party or other entity. Such entities may outsource data for analysis, for example, when they have a large volume of data that they are unable to analyze themselves. In such situations, the entity may analyze some of this data and outsource the remaining data to the third-party or other entity for analysis. At a high-level, a first entity 1122 (which may be first system device 1102 of FIG. 36, for example) may produce the ECG data and upload the data to system 1124 (which may be server 1101 of FIG. 36, for example). Second entity 1126 (which may be second system device 1105 of FIG. 36, for example) may then access the uploaded ECG data, provide an indication to system 1124 to perform an analysis on the data, and system 1124 may then generate a report based on the analysis. The report may then be accessed by first entity 1122 from system 1124. In some scenarios, system 1124 may automatically provide an indication that the ECG data has been uploaded to second entity 1126 upon upload and/or the report may automatically be provided to the first entity 1122. In one example, pharmacies may have the ability to obtain ECG data from a patient and provide a report to the patient, but such pharmacies may rely on a third-party or other entity to analyze the ECG data and generate the report that is provided to the patient.

Turning to process 1170, at operation 1171, computer-executable instructions stored on a memory of a device, such as a computing device, may be executed to modify and/or access administrative information to modify or grant access and/or authorizations to various folders, data and/or functionality on system 1124. Likewise, system 1124 may receive an indication of a modification or grant from first entity 1122. For example, first entity 1122 may indicate, using system 1124, which users are able to perform certain actions with respect to any of the data uploaded to the system 1124. In one example, system 1124 may have a number of folders and first entity 1122 may grant second entity 1126 access to one or more folders, data and/or functionality including the relevant uploaded ECG data and/or any other data. Second entity 1126 may not have permissions to access other data uploaded by first entity 1122 to system 1124 and/or other data on system 1124. For example, there may exist a second folder including other ECG data for other patients and/or any other type of data. First entity 1122 may grant second entity 1126 in the first folder, but not the second folder. Additional examples of user rights may be presented below with respect to FIG. 41.

At operation 1172, computer-executable instructions stored on a memory of a device, such as a computing device, may be executed to modify or grant access to administrative information and/or authorizations to various folders, data and/or functionality on system 1124. Likewise, system 1124 (for example, server 1101) may receive an indication of a grant or modification from second entity 1126. The authorizations granted by second entity 1126 may be the same or different from those granted by first entity 1122.

At operation 1173, computer-executable instructions stored on a memory of a device, such as a computing device, may be executed to upload ECG data. For example, first entity 1122 may capture and/or obtain ECG data from a user (for example, user 1104 in FIG. 36) and may upload the captured data to system 1124. Likewise, system 1124 may receive the uploaded data from first entity 1122. The data may, for example, be uploaded into a first folder that second entity 1126 has permission to access. These permissions may be established by first entity 1122 (for example, as described with respect to operation 1171) such that second entity 1126 may obtain the uploaded data for analysis purposes.

At operation 1174, which may be optional, computer-executable instructions stored on a memory of a device, such as a computing device, may be executed to send an indication for an analysis to be performed by system 1124. For example, first entity 1122 may indicate to system 1124 to perform any analyses on the uploaded ECG data. The ECG data may be analyzed in accordance with the ECG processing system 10 described with respect to FIG. 4 and/or may be analyzed based on any other type of analysis using any other system and/or device mentioned herein. Likewise, system 1124 may receive the indication to perform any analyses and may perform the analyses. Alternatively, or additionally, at operation 1175, second entity 1122 may indicate to system 1124 to perform any analyses on the uploaded ECG data. At optional operation 1175, computer-executable instructions stored on a memory of a device, such as a computing device, may be executed to send an indication to perform any analyses of the uploaded ECG data. For example, second entity 1126 may provide the indication and system 1124 may then perform any analyses based on the indication. The analyses performed may be performed based on either an indication from first entity 1122 or second entity 1126 based on if the analysis is outsourced to second entity 1126 or remains with first entity 1122. It is understood that operation 1175 may be optional and that system may automatically initiate operation 1176. At operation 1176, computer-executable instructions stored on a memory of a device, such as a computing device, may be executed to analyze the ECG data. That is, system 1124 may perform any analyses (e.g., based on the indication provided by first entity 1122 in operation 1174). At operation 1177 second entity 1126 may access ECG data on system 1124 (e.g., that second entity 1126 has permission to access). For example, system 1124 may receive a request to access certain data and may permit second entity 1126 to access such data. It is understood that operation 1177 may occur in a different order (e.g., before operation 1176).

At operation 1178, computer-executable instructions stored on a memory of a device, such as a computing device, may be executed to prepare a report based on the analysis of the ECG data performed by system 1124 in operation 1176. The report may include any information relevant to the analysis of the ECG data. For example, the report may indicate whether any medical anomalies, conditions, and/or events exist in the ECG data.

At operation 1179, computer-executable instructions stored on a memory of a device, such as a computing device, may be executed to access the report generated by system 1124. That is, first entity 1122 may access the generated report. At operation 1180, computer-executable instructions stored on a memory of a device, such as a computing device, may be executed to access the report generated by system 1124. That is, second entity 1126 may access the generated report.

Referring now to FIG. 38B, exemplary process 1190 for ECG analyses and restricting access to ECG data and/or analyses is depicted. For example, process 1190 may be directed to a process by which entities that produce ECG data (for example, first entity 1122 in FIG. 37A) interact with a system (for example, system 1124 in FIG. 37A) themselves to initiate an analysis of the ECG data to produce a report, but may occasionally may outsource the management of the analysis to a third-party (for example, second entity 1126 in FIG. 37A) or other entity. Some or all of the blocks of the process flows in this disclosure may be performed in a distributed manner across any number of devices (e.g., servers, computing devices, user devices and/or servers). Some or all of the operations of the process flow may be optional and may be performed in a different order. It is understood that the process in FIG. 38B may be performed by a server (e.g., server 1101 of FIG. 36).

At operation 1191, computer-executable instructions stored on a memory of a device, such as a computing device, may be executed to receive authorization instructions indicative of access rights. For example, the second entity may indicate, using the system, which users are able to upload data to a particular location within the system. Any other types of user rights may also be indicated. This location within the system, for example, may be represented by a number of folders presented through a user interface associated with system. However, the storage may be depicted in any other format as well. In this manner, it should be noted that any reference to a “folder” herein may simply indicate a portion of system that is reserved for storage by any entities that have permission to use this storage space. Particularly, the second entity may grant the first entity access to a folder in which any ECG data may be located. However, first entity may not have permission to access other data uploaded to the system in other locations. Additional examples of user rights may be presented FIG. 41. The second entity may have any number of different folders included within the system that may be intended for any number of different entities. For example, the system may facilitate analyses of ECG data received from any number of different entities. A first folder may be reserved for the first entity, a second folder may be reserved for a third entity, etc. This may allow each of the entities to upload ECG data to their respective folders within the system, while preventing an entity from accessing the data saved to folders for which such entity does not have authorization to access.

In some embodiments, the data may also be uploaded to one or more folders associated with the first entity instead of the second entity. That is, the first entity may manage the one or more folders as an administrator and may indicate permissions to access the folder. This may provide administrative control to the first entity that is uploading the ECG data rather than the second entity that is facilitating the analysis of the ECG data. For example, the first entity may upload any ECG data into its own folders if it is managing the analysis process itself. In such situations, the second entity may not be involved in the process and may not need access to the ECG data.

At operation 1192, computer-executable instructions stored on a memory of a device, such as a computing device, may be executed to receive uploaded ECG data. For example, the first entity may capture and/or otherwise obtain ECG data from a user (for example, user 1104 in FIG. 36) and may upload the captured data to the system. Particularly, the first entity may be associated with a first system device, which may receive data captured from a user by an ECG sensing device. The system may receive this uploaded data from the first entity. The data may, for example, be uploaded into a first folder that second entity has permission to access. These permissions may be established by the first entity and/or by the second entity (for example, as described with respect to operation 1171 and/or operation 1172 of FIG. 38A) such that the second entity may obtain the uploaded data for analysis purposes.

Optional operations 1194-1196 may include operations that may be performed if the first entity is outsourcing management of the analysis of the uploaded ECG data to second entity. At optional operation 1194, computer-executable instructions stored on a memory of a device, such as a computing device, may be executed to automatically provide an indication that the ECG data was uploaded by the first entity. That is, the system may be configured to automatically provide a notification to the second entity when new data is uploaded into a folder that the second entity has permissions to access. This notification may be provided in scenarios where the first entity is outsourcing the management of the analysis of the ECG data to the second entity. In some embodiments, these automatic notifications may be configured in settings associated with the system by the first entity and/or the second entity. For example, the second entity may prefer to receive notifications so that the second entity may have knowledge of when new data is uploaded for which the second entity may initiate an analysis via the system (for example, when ECG data produced by the first entity is outsourced to the second entity to handle the analysis as aforementioned). The automatic notifications may be provided in any well-known manner, such as a phone call, email communication, text message, and/or the like. The automatic notifications may also be provided within the system (for example, presented through a user interface) and/or through the use of an application programming interface (API).

At optional operation 1195, computer-executable instructions stored on a memory of a device, such as a computing device, may be executed to receive a request to access the uploaded ECG data. For example, the uploaded data may be stored within the system, and the second entity may need to access the data in order to facilitate the analysis. The second entity may access this data, for example, through a user interface associated with the system that may be presented to the second entity through the system. The second entity may access a location in which the data is stored within system using the user interface. The system may confirm that second entity is authorized to view the data. If it is determined that second entity is authorized to view the data, then the data may be presented to second entity through the user interface. At optional operation 1196, computer-executable instructions stored on a memory of a device, such as a computing device, may be executed to receive an indication to perform an analysis of the uploaded ECG data. For example, the indication may come from the second entity.

As an alternative to operations 1194-1196 and/or in addition to operations 1194-1196, the first entity that provided the ECG data may send an indication (e.g., request, instructions and/or command) to perform analysis on the uploaded ECG data and at operation 1193 computer-executable instructions stored on a memory of a device, such as a computing device, may be executed to receive the indication to perform analysis on the uploaded ECG data. The first entity send this request operation if the first entity is managing the analysis itself. Thus, process 1190 may proceed through operation 1193 instead of optional operations 1194-1196 if first entity 1122 is managing the analysis.

At operation 1197, computer-executable instructions stored on a memory of a device, such as a computing device, may be executed to perform the analysis of the uploaded ECG data. For example, the analysis may include any processes described with respect to FIG. 4 and/or any other types of possible analyses of ECG data described herein. Such analyses, for example, may be used to identify abnormalities, events and/or conditions of a patient. Abnormalities, events and/or conditions may include but are not limited to, sinoatrial block, paralysis or arrest, atrial fibrillation, atrial flutter, atrial tachycardia, junctional tachycardia, supraventricular tachycardia, sinus tachycardia, ventricular tachycardia, pacemaker, premature ventricular complex, premature atrial complex, first degree atrio-ventricular block (AVB), 2nd degree AVB Mobitz I, 2nd degree AVB Mobitz II, 3rd degree AVB, Wolff-Parkinson-White syndrome, left bundle branch block, right bundle branch block, intraventricular conduction delay, left ventricular hypertrophy, right ventricular hypertrophy, acute myocardial infarction, old myocardial infarction, ischemia, hyperkalemia, hypokalemia, brugada, and/or long QTc.

At operation 1198, computer-executable instructions stored on a memory of a device, such as a computing device, may be executed to generate an analysis report and/or output data corresponding to analysis of the uploaded ECG data. The report and/or output data may include any information relevant to the analysis of the ECG data. For example, the report and/or output data may indicate whether any anomalies, events and/or conditions exist in the patient's ECG data. The report and/or output data may also include any other types of information, such as an illustration of the ECG data, an indication of different points of interest in the data, any data classifications that were performed, and/or any other information that may be relevant to the analysis. The report and/or output may be generated by the system. The report and/or output data may also be stored on the system, such that it may be accessed at a later time (for example, accessed by the first entity). In some cases, the report and/or output data may be stored in the same folder including the uploaded ECG data. However, the report may also be stored in any other location as well.

At operation 1199, computer-executable instructions stored on a memory of a device, such as a computing device, may be executed to receive a request to access and/or generation of the analysis report (e.g., from the first entity and/or second entity). At operation 1200, computer-executable instructions stored on a memory of a device, such as a computing device, may be executed to determine authorization to grant access to analysis report based on authorization instructions. For example, the system may determine if the first entity or the second entity has authorization to access the location within the system in which the report is stored and/or if the first or second entity has authorization to generate an analysis report based on the output data. A further authorization may also be required to access the report. The report may be saved on the system and/or may be generate and communicated to another device without being saved on the system.

At operation 1201, computer-executable instructions stored on a memory of a device, such as a computing device, may be executed to provide the analysis report (e.g., to the first entity and/or second entity). For example, the first entity may be able to view the report through a user interface associated with system. System may also allow first entity to download the report to a local device for local storage and/or viewing. However, in some cases, system may prevent any users from downloading the report, and rather may maintain the report within system for viewing.

At optional operation 1202, computer-executable instructions stored on a memory of a device, such as a computing device, may be executed to automatically provide the analysis report (e.g., to the first entity and/or second entity). For example, similar to the optional automatic notifications provided to a second entity when data is uploaded, automatic notifications may also be provided to first entity when an analysis report is generated. These automatic notifications may be configured in settings associated with the system by the first entity and/or the second entity (and/or may not require any configuration by either entity). These automatic notifications may allow first entity to be notified when an analysis has been completed and a report has been generated without having to access system to check to determine if the report has been generated. The automatic notifications may also be provided in any suitable manner, such as a phone call, email communication, text message, and/or the like. The automatic notifications may also be provided within the system (for example, presented through a user interface) and/or through the use of an application programming interface (API).

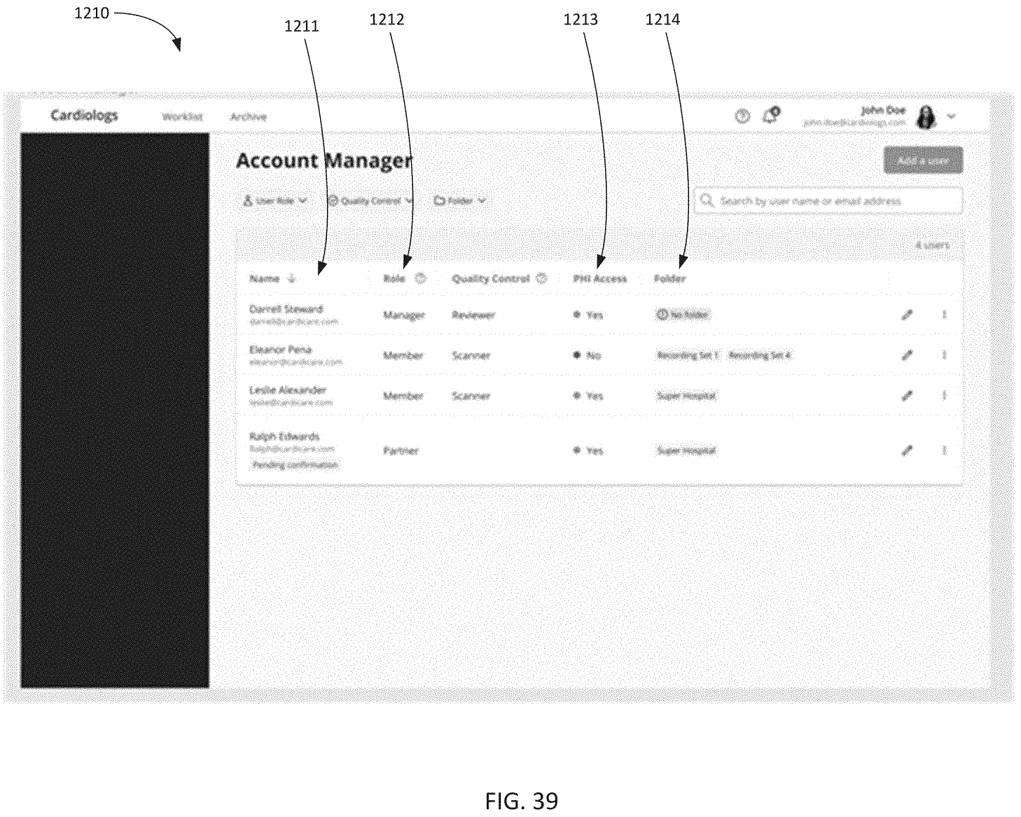

FIG. 39 is an exemplary user interface 1210 of an account manager. User interface 1210 may present an example of a user interface that may be presented to a user (such as user 1106 and/or user 1107 of FIG. 36). The account manager interface may allow an administrator associated with a first entity to manage user permissions on the system. The administrator may be able to configure which users are able to view and/or access data included in particular folders. For example, of the account manager interface may include a table including user name data 1211, role data 1212, access data 1213, and folder data 1214. User name data 1212 may indicate a particular user and role data 1212 may provide information about that user (e.g., manager). Access data 1213 may indicate certain access permissions for that user. Folder data 1214 may indicate one or more folders for which a user as certain access permission or rights.

The users illustrated in the figure may be associated with different entities. A first user may be associated with a second entity and may be granted permission to access a first folder (not illustrated in the figure). The first entity may then be able to upload data (such as a patient's ECG data) into the first folder and the first user may be able to access the data from the first folder for analysis. The data in this folder may be downloaded by the first user. Alternatively, the first user may not be allowed to download the data, but may still be able to access and view the data. The first user may also be restricted from accessing data included in other folders associated with the first entity. In this manner, the first entity may be able to upload data for access by another entity, but may retain the privacy associated with any other data that is not intended for the other entity.

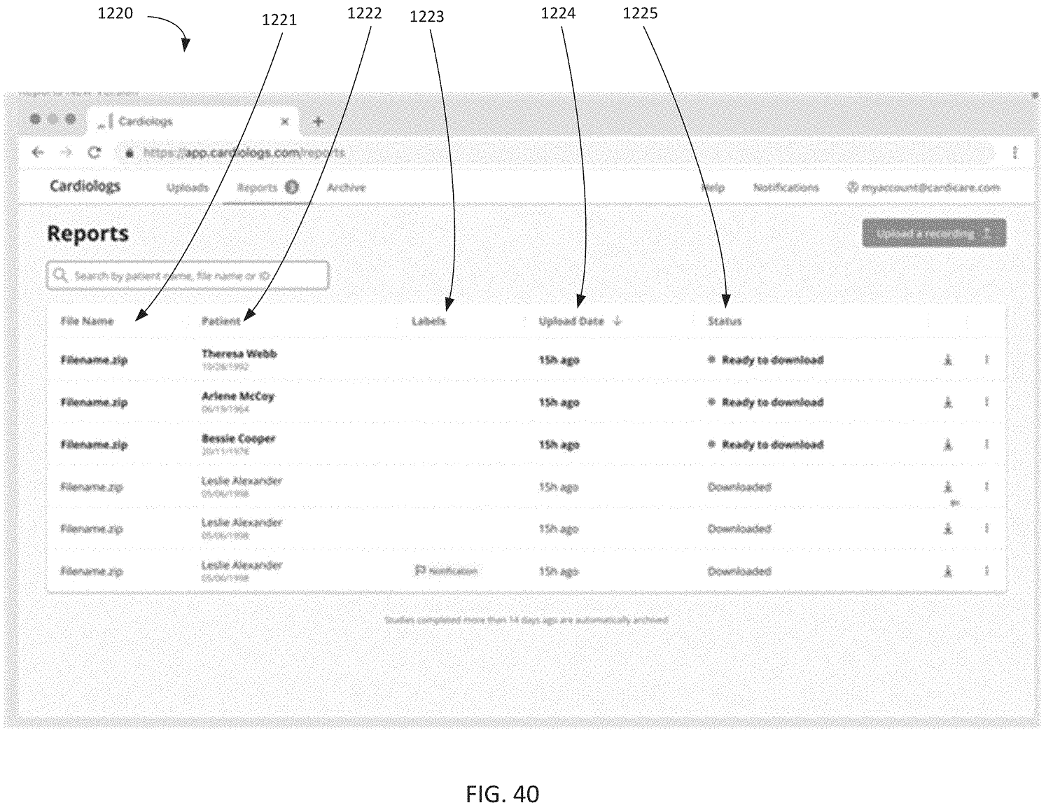

FIG. 40 is an exemplary user interface 1220 for displaying report information. For example, user interface 1220 may illustrate various reports that are saved to and/or uploaded to the server. These reports may be accessed and/or provided to a patient and/or any other user. As shown in FIG. 40, user interface 1220 may include a file name data 1221, patient data 1222, label data 1223, upload data 1224, and status data 1225. File name data 1221 may include the file name for the report that is saved to the system. Patient data 1222 may include the patient's name and/or identification value. Label data 1223 may include one or more labels identified in the respective report corresponding to patient data 1222. Upload data 1224 may indicate the upload data for the respective report. Status data 1224 may include a download status for a given report (e.g., ready to download or downloaded).

It should be noted that any other types of user interfaces may be presented to the user and user interface 1210 and user interface 1220 are not intended to be limiting in any way. For example, a user 1107 may be presented with a user interface including a listing of folders that the user 1107 has permission to access. A user 1106 may be presented with a user interface that includes all of the folders associated with the user 1106. Any other user interfaces may be presented that may allow any user to perform any of the functionalities described herein as well.

FIG. 41 is an exemplary user interface 1230 for granting and/or modifying certain rights and/or authorizations. User interface 1230 may exemplify a number of different toggles that an administrator may control to limit certain rights of different users (for example, any users that access an analysis system, such as server 1101 of FIG. 36, and/or any other analysis system described herein). For example, toggle 1231 may include access rights to particular folders that may be associated with an identifier, such as a folder name or user identification. Toggle 1232 may include upload rights to particular folders. Toggle 1233 may include view access rights for certain files in any folders. Toggle 1234 may include access rights to any data analyses (e.g., any data analysis described throughout this application). Toggle 1235 may include access rights to view any report(s). Toggle 1236 may include access rights to revise and/or comment on any report(s). Toggle 1237 may include access rights to generate any report(s). Toggle 1238 may include access rights to view and/or revise administrator information. Toggle 1238 may include access rights for a user and/or folder based on duration (e.g., access rights to a user or folder may be based on a set amount of time). For example, a certain user may have access to a certain folder for only a set period of time. It is understood that these are merely examples of certain user rights that may be restricted, and any other types of user rights may also be applicable. It is further understood that only one or more of these rights and/or authorizations may be included in user interface 1230. Additionally, the use of the on and/or off toggles in the user interface are merely exemplary and user rights may be adjusted using any other suitable method (e.g., checkboxes, selecting items from a list, etc.).

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A computerized-method for analyzing electrocardiogram (ECG) data of a patient and restricting access to analyzed ECG data, the computerized-method comprising:
receiving, by a server, authorization instructions corresponding to a first location on the server, a first account, and a second account;
receiving a set of patient ECG data from the first account;
storing the set of patient ECG data at the first location on the server based on the authorization instructions;
processing at least a portion of the set of patient ECG data using an algorithm to determine a presence of one or more abnormalities, conditions, or descriptors corresponding to a cardiac event associated with the set of patient ECG data, the algorithm trained using a plurality of sets of ECG data different from the set of ECG data;
generating output data based on the presence of the one or more abnormalities, conditions, or descriptors;
storing the output data at the first location based on the authorization instructions;
receiving a request to access the output data from the second account; and
permitting the second account to access one or more of the set of patient ECG data and the output data based on the authorization instructions.

2. The computerized-method of claim 1, wherein the authorization instructions are received from the second account.

3. The computerized-method of claim 1, wherein the authorization instructions comprise at least one of: authorization to access the first location, authorization to upload data to the first location, authorization to access a first type of data within the first location, authorization to access or revise administrative information, authorization to view the output data, authorization to revise the output data, or authorization to generate a report based on the output data.

4. The computerized-method of claim 3, wherein the first location is a first digital folder, the computerized-method further comprising:
determining the second account has authorization to access the first digital folder based on the authorization instructions; and
permitting, based on determining the second account having authorization to access the first folder, the second account access to the set of patient ECG data in the first digital folder.

5. The computerized-method of claim 1, further comprising:
automatically processing the set of patient ECG data using the algorithm upon receiving the set of patient ECG data.

6. The computerized-method of claim 1, further comprising:
receiving, from the second account, a request to process the set of patient ECG data using the algorithm.

7. The computerized-method of claim 1, further comprising:
receiving, from the second account, a request to generate a report based on the output data.

8. The computerized-method of claim 7, further comprising:
determining the second account has authorization to request the report be generated based on the authorization instructions.

9. The computerized-method of claim 1, further comprising:
sending, upon receiving the set of patient ECG data and storing the patient ECG data at the first location, a message to the second account indicating that the set of patient ECG data is saved at the first location.

10. The computerized-method of claim 1, further comprising:
receiving a request from one or more of the first account and the second account to view the output data; and
granting the request to view the output data based on the authorization instructions.

11. The computerized-method of claim 1, further comprising:
receiving a request to modify the output data from one or more of the first account and the second account; and
granting the request to modify the output data based on the authorization instructions.

12. A system for analyzing electrocardiogram (ECG) data of a patient and restricting access to analyzed ECG data, the system comprising:
memory configured to store computer-executable instructions, and
at least one computer processor configured to access memory and execute the computer-executable instructions to:
receive authorization instructions corresponding to a first location on the at least one computer processor, a first account, and a second account;
receive a set of patient ECG data from the first account;
store the set of patient ECG data at the first location based on the authorization instructions;
process at least a portion of the set of patient ECG data using an algorithm to determine a presence of one or more abnormalities, conditions, or descriptors corresponding to a cardiac event associated with the set of patient ECG data, the algorithm trained using a plurality of sets of ECG data different from the set of ECG data;
generate output data based on the presence of the one or more abnormalities, conditions, or descriptors;
store the output data at the first location based on the authorization instructions;
receiving a request to access the output data from the second account; and
permit the second account to access one or more of the set of patient ECG data and the output data based on the authorization instructions.

13. The system of claim 12, wherein the authorization instructions are received from the second account.

14. The system of claim 12, wherein the authorization instructions comprise at least one of: authorization to access the first location, authorization to upload data to the first location, authorization to access a first type of data within the first location, authorization to access or revise administrative information, authorization to view the output data, authorization to revise the output data, or authorization to generate a report based on the output data.

15. The system of claim 14, wherein the first location is a first digital folder and the at least one computer processor is further configured to access memory and execute the computer-executable instructions to:
determine the second account has authorization to access the first digital folder based on the authorization instructions; and
permit, based on determining the second account having authorization to access the first folder, the second account access to the set of patient ECG data in the first digital folder.

16. The system of claim 12, wherein the at least one computer processor is further configured to access memory and execute the computer-executable instructions to:
automatically process the set of patient ECG data using the algorithm upon receiving the set of patient ECG data.

17. The system of claim 12, wherein the at least one computer processor is further configured to access memory and execute the computer-executable instructions to:
receive, from the second account, a request to process the set of patient ECG data using the algorithm.

18. The system of claim 12, wherein the at least one computer processor is further configured to access memory and execute the computer-executable instructions to:
receive, from the second account, a request to generate a report based on the output data.

19. The system of claim 18, wherein the at least one computer processor is further configured to access memory and execute the computer-executable instructions to:
determine the second account has authorization to request the report be generated based on the authorization instructions.

20. The system of claim 12, wherein the at least one computer processor is further configured to access memory and execute the computer-executable instructions to:
send, upon receiving the set of patient ECG data and storing the patient ECG data at the first location, a message to the second account indicating that the set of patient ECG data is saved at the first location.
